(19) United States
(12) Patent Application Publication
(10) Pub. No.: US 2024/0424022 A1
(43) Pub. Date: Dec. 26, 2024

(54) IN VIVO AND EX VIVO METHODS OF MODULATING T CELL EXHAUSTION/DE-EXHAUSTION

(71) Applicant: Flagship Pioneering Innovations VI, LLC, Cambridge, MA (US)

(72) Inventors: Fabian Alexander WOLF, Munich (DE); John BRADLEY, Scitcuate, MA (US); Xiaoji SUN, Cambridge, MA (US); Nicholas McCartney PLUGIS, Boston, MA (US)

(21) Appl. No.: 18/687,668

(22) PCT Filed: Sep. 1, 2022
(86) PCT No.: PCT/US2022/042349

§ 371 Date: Feb. 28, 2024

Related U.S. Application Data
(60) Provisional application No. 63/239,828, filed on Sep. 1, 2021.
(60) Provisional application No. 63/239,832, filed on Sep. 1, 2021.
(60) Provisional application No. 63/277,375, filed on Nov. 9, 2021.

(51) Int. Cl. A61K 35/17; A61P 35/00; G01N 33/50

(52) U.S. Cl. CPC A61K 35/17; A61P 35/00; G01N 33/505

(57) ABSTRACT

The present disclosure relates, inter alia, to perturbagens and methods, including ex vivo methods, for directing a change in the cell state of T cells, e.g., naïve T cells, effector T cells and exhausted T cells. The present disclosure also relates to methods for mitigating or preventing T cell exhaustion, including contacting cells with a perturbagen ex vivo. Further, the present disclosure relates to methods for treating diseases or disorders characterized by, e.g., production of exhausted T cells, production of abnormal number of exhausted T cells, or production of high number of T cells.

### CROSS-REFERENCE TO RELATED APPLICATIONS

This application claims priority to and benefit of U.S. Provisional Patent Application No. 63/239,828 filed on Sep. 1, 2021, U.S. Provisional Patent Application No. 63/277,375 filed on Nov. 9, 2021, and U.S. Provisional Patent Application No. 63/239,832 filed on Sep. 1, 2021, the contents of all of which are hereby incorporated by reference in their entireties.

### BACKGROUND

Prolonged exposure of T cells to antigens (such as tumor antigens or viral antigens) can lead to a deterioration of T cell function, often referred to as T cell exhaustion. T cell exhaustion is associated with reduced ability of the immune system to control tumor growth and chronic infections. Exhausted T cells typically exhibit increased expression of inhibitory receptors, decreased production of effector cytokines, decreased proliferation rates, and decreased target cell killing activity. Revitalization of exhausted T cells can reinvigorate immunity. There is a need in the art for new and improved methods that can be used to reduce or reverse T cell exhaustion, and to screen for new therapeutic candidates or study the effects of known or new therapeutic agents on T cell exhaustion. The present disclosure addresses these and other needs.

### SUMMARY

In aspects and embodiments, there is provided a method for directing a change in cell state of a T cell in a subject, comprising, administering at least one perturbagen selected from Table 3, or a variant thereof to the subject, the perturbagen being capable of altering a gene signature in the T cell.

In aspects and embodiments, there is provided a method for directing a change in cell state of a T cell in a subject, comprising, administering at least one perturbagen capable of altering a gene signature in the T cell to the subject, wherein altering the gene signature comprises an increase in expression and/or activity in the T cell of one or more genes selected from Table 1 designated as an “up” gene in the gene directionality column of Table 1 and/or a decrease in expression and/or activity in the T cell of one or more genes selected from Table 1 designated as a “down” gene in the gene directionality column of Table 1.

In embodiments, altering the gene signature comprises an activation of a network module designated in the network module column of Table 1.

In aspects and embodiments, there is provided a method for directing a change in cell state of a T cell in a subject, comprising, administering at least one perturbagen selected from Table 3, or a variant thereof to the subject, the perturbagen being capable of altering a gene signature in the T cell, wherein altering the gene signature comprises an increase in expression and/or activity in the T cell of one or more genes selected from Table 1 designated as an “up” gene in the gene directionality column of Table 1 and/or a decrease in expression and/or activity in the T cell of one or more genes selected from Table 1 designated as a “down” gene in the gene directionality column of Table 1.

In embodiments, altering the gene signature comprises an activation of a network module designated in the network module column of Table 1.

In aspects and embodiments, there is provided an ex-vivo method for directing a change in cell state of a T cell, comprising, contacting a population of cells with at least one perturbagen selected from Table 4, or a variant thereof, the perturbagen being capable of altering a gene signature in the T cell.

In aspects and embodiments, there is provided an ex-vivo method for directing a change in cell state of a T cell, comprising, contacting a population of cells with at least one perturbagen capable of altering a gene signature in the T cell, wherein altering the gene signature comprises an increase in expression and/or activity in the T cell of one or more genes selected from Table 2 designated as an “up” gene in the gene directionality column of Table 2 and/or a decrease in expression and/or activity in the T cell of one or more genes selected from Table 2 designated as a “down” gene in the gene directionality column of Table 2.

In aspects and embodiments, there is provided an ex-vivo method for directing a change in cell state of a T cell, comprising, contacting a population of cells with at least one perturbagen selected from Table 4, or a variant thereof, the perturbagen being capable of altering a gene signature in the T cell, wherein altering the gene signature comprises an increase in expression and/or activity in the T cell of one or more genes selected from Table 2 designated as an “up” gene in the gene directionality column of Table 2 and/or a decrease in expression and/or activity in the T cell of one or more genes selected from Table 2 designated as a “down” gene in the gene directionality column of Table 2.

In embodiments, the population of cells is derived from a subject, optionally, from the subject's bone marrow or the subject's blood, and/or the population of cells is fractionated to obtain fractionated cells prior to contacting with at least one perturbagen, optionally, wherein the fractionated cells are enriched for T cells.

In embodiments, the method further comprises the step of administering a resultant population of cells to a subject wherein resultant population of cells is obtained upon contacting the population of cells with at least one perturbagen, optionally, wherein the subject is the source of the population of cells to which perturbagen is contacted or the subject is not the source of the population of cells to which perturbagen is contacted, and/or the method further comprises contacting the population of cells with one or more of IL-2, an antigen, and an antigen-presenting cell.

In embodiments, altering the gene signature comprises an activation of a network module designated in the network module column of Table 2.

In embodiments, the T cell is selected from an effector T cell, an exhausted T cell, and a naïve T cell.

In embodiments, the change in cell state is a prevention or reduction of a cellular transition to an exhausted T cell state, or a prevention or reduction of a transition of an effector T cell to an exhausted T cell state, or a prevention or reduction of a transition of a naïve T cell to an exhausted T cell state, or a stimulation or increase of a transition of an exhausted T cell to an effector T cell, or a stimulation or increase of a transition of a naïve T cell to an effector T cell, or a stimulation or increase of a cell death of an exhausted T cell, or a prevention or reduction of a cell death of an effector T cell and/or a naïve T cell.

In embodiments, the change in cell state provides an increase in the number of one or more of effector T cells and naïve T cells, or an increase in the number of one or more of effector T cells and naïve T cells relative to the number of effector T cells or naïve T cells obtained from a population of T cells that is not contacted with the at least one perturbagen, or an increase in the number of one or more of effector T cells and naïve T cells relative to the number of effector T cells or naïve T cells obtained from a population of T cells prior to contacting with the at least one perturbagen, or a decrease in the number of one or more of exhausted T cells, or a decrease in the number of one or more of exhausted T cells relative to the number of exhausted T cells obtained from a population of T cells that is not contacted with the at least one perturbagen, or a decrease in the number of one or more of exhausted T cells relative to the number of exhausted T cells obtained from a population of T cells prior to contacting with the at least one perturbagen.

In embodiments, the number of effector T cells and/or naïve T cells is increased after contacting the population of T cells with the at least one perturbagen.

In embodiments, the number of exhausted T cells is decreased after contacting the population of T cells with the at least one perturbagen.

In embodiments, the method promotes the formation of T cells that are responsive to antigen, the antigen optionally being presented by a professional antigen-presenting cell, and/or T cells that are responsive to both TCR engagement and co-stimulation, and/or T cells that are capable of elaborating cytokines, and/or T cells that are capable of proliferating, e.g. in the presence of TCR engagement and co-stimulation, or T cells that are anabolic, or T cells that are cytotoxic, or effector T cells, or T cells that are not responsive or poorly responsive to antigen, optionally wherein the antigen is being presented by a professional antigen-presenting cell.

In embodiments, the method represses the formation of T cells that are not responsive or poorly responsive to both TCR engagement and co-stimulation, or T cells that are incapable or poorly capable of elaborating cytokines, or the formation of T cells that are incapable or poorly capable of proliferating, e.g. in the presence of signal 1 and signal 2, and/or the formation of T cells that are not anabolic or minimally anabolic, or T cells that are not cytotoxic or minimally cytotoxic, and/or exhausted T cells. In embodiments, the T cells are CD8+ T cells and/or CD4+ T cells.

In embodiments, at least one perturbagen selected from Table 3, or a variant thereof, comprises at least 2, at least 3, at least 4, or at least 5, at least 6, at least 7, at least 8, at least 9, at least 10, at least 11, at least 12, at least 13, at least 14, at least 15, at least 16, at least 17, at least 18, at least 19, and at least 20 perturbagens selected from Table 3, or variants thereof.

In embodiments, one or more genes selected from Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, and 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more genes are selected from Table 1 designated as an “up” gene in the gene directionality column of Table 1.

In embodiments, one or more genes selected from Table 1 comprises at least one of NFKBIA, TSC22D3, ZFP36, ARHGEF2, FHL2, STMN1, CDC25B, CCND3, TMEM109, E2F2, SCP2, PDLIM1, CORO1A, ATP11B, SATB1, CXCR4, ARL4C, CTSD, CD44, ZMIZ1, TBXA2R, GNA15, PRKCQ, RHOA, SLC25A4, PRUNE, CDC42, TIMP2, FAM69A, NRAS, BHLHE40, DNAJC15, GNAI2, DHRS7, CYTH1, ADGRE5, IGF2R, ADRB2, EIF4EBP1, FAS, MRPS16, TMEM50A, S100A4, RSU1, SPTAN1, S100A13, RAC2, REEP5, MACF1, PLP2, and TWF2.

In embodiments, one or more genes selected from Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, and 20 or more, 21 or more, 22 or more, 23 or more, 24 or more, 25 or more, 26 or more, 27 or more, 28 or more, 29 or more, 30 or more, or 31 or more genes selected from Table 1 designated as a “down” gene in the gene directionality column of Table 1.

In embodiments, one or more genes selected from Table 1 comprises at least one of STAT1, DUSP6, INPP1, PSMB8, MLEC, ID2, RGS2, UBE2L6, SSBP2, PRKCH, ALDOA, ADGRG1, MFSD10, HERC6, CEP57, FBXL 12, ICAM1, GLRX, PSME2, MYCBP2, IKZF1, PSMB10, PSME1, EVL, MBNL1, FYN, DNAJB6, FOXO3, TSPAN3, SYNE2, and RPS6.

In embodiments, at least one perturbagen selected from Table 4, or a variant thereof, comprises at least 2, at least 3, at least 4, or at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35, at least 36, at least 37, at least 38, at least 39, at least 40, at least 41, at least 42, or at least 43 perturbagens selected from Table 4, or variants thereof.

In embodiments, one or more genes selected from Table 2 designated as an “up” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, and 19 or more, 20 or more, 21 or more, 22 or more, 23 or more, 24 or more genes selected from Table 2 designated as an “up” gene in the gene directionality column of Table 2.

In embodiments, one or more genes selected from Table 2 comprises at least one of MYC, TES, CXCR4, IGFBP3, PRSS23, SYPL1, CYB561, CCNH, XBP1, RPS6, ADRB2, GDPD5, SORBS3, ZFP36, FOS, PXN, SLC25A4, DSG2, SATB1, IER3, SSBP2, RPS5, ATP1B1, and GADD45B.

In embodiments, one or more genes selected from Table 2 designated as a “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, and 62 or more genes selected from Table 2 designated as a “down” gene in the gene directionality column of Table 2.

In embodiments, one or more genes selected from Table 2 comprises at least one of CDK6, MTHFD2, ID2, SCCPDH, SLC25A46, ETFB, HLA-DRA, CHN1, RAB27A, TBXA2R, NFKB2, ITGAE, SMC4, STMN1, GATA3, ETS1, IQGAP1, CAT, RALA, TSC22D3, CBLB, INPP4B, PLSCR1, NUSAP1, RGS2, EVL, PSMB8, HERPUD1, APBB2, MIF, SQSTM1, PGAM1, TWF2, DRAP1, ETV1, CCNA1, HTRA1, DUSP4, GAPDH, RPA3, ADGRG1, ACOT9, CALM3, SOX4, HMOX1, RHOA, S100A4, ANKRD10, FCHO1, KDM5B, SPTAN1, CTSD, HLA-DMA, FGFR4, SLC1A4, HSPB1, CDKN2A, STAT3, RAC2, TIAM1, RALGDS, and EZH2.

In aspects and embodiments, there is provided a perturbagen for use in a method disclosed herein.

In aspects and embodiments, there is provided a pharmaceutical composition comprising a perturbagen disclosed herein.

In aspects and embodiments, there is provided a method for treating a disease or disorder characterized by insufficient T cell response, comprising administering to a subject in need thereof a therapeutically effective amount of at least one perturbagen selected from Table 3, or a variant thereof, wherein the at least one perturbagen is capable of changing a gene signature in a T cell.

In aspects and embodiments, there is provided a method for treating a disease or disorder characterized by insufficient T cell response, comprising administering to a subject in need thereof a population of cells, the population of cells having been contacted with at least one perturbagen selected from Table 4, or a variant thereof, wherein the at least one perturbagen is capable of changing a gene signature in a T cell.

In embodiments, the disease or disorder is characterized by immune tolerance. In embodiments, the disease or disorder characterized by insufficient T cell response is a cancer. In embodiments, the cancer is a solid tumor or a liquid tumor.

In embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In embodiments, the cancer is a melanoma, optionally selected from metastatic melanoma, cutaneous melanoma, BRAF V600, Merkel cell carcinoma, cutaneous squamous cell carcinoma, a lung cancer, optionally selected from NSCLC, metastatic nonsquamous non-small cell lung cancer, metastatic small cell carcinoma, a head and neck cancer, optionally selected from head and neck squamous cell cancer, a bladder cancer, optionally selected from urothelial carcinoma, locally advanced or metastatic urothelial carcinoma, a breast cancer, optionally selected from triple-negative breast cancer), microsatellite Instability-high cancer, gastric cancer, optionally selected from gastric or gastroesophageal junction adenocarcinoma, metastatic colorectal cancer, cervical cancer, optionally selected from recurrent or metastatic cervical cancer, liver cancer, optionally selected from HCC, hematological disorder, optionally selected from primary mediastinal large B-cell lymphoma, and Hodgkin lymphoma.

In embodiments, the subject is selected by steps comprising, obtaining from the subject a sample of cells comprising a T cell; and contacting the sample of cells with least one perturbagen selected from Table 3 or Table 4, or a variant thereof, wherein the at least one perturbagen alters a gene signature in the sample of cells.

In embodiments, the subject is selected by steps comprising, obtaining from the subject a sample of cells comprising a T cell, and contacting the sample of cells with at least one perturbagen capable of altering a gene signature in a T cell, wherein the at least one perturbagen increases in the sample of cells the expression and/or activity of one or more genes selected from Table 1 or Table 2 designated as an “up” gene in the gene directionality column of Table 1 or Table 2 and/or decreases in the sample of cells the expression and/or activity of one or more genes selected from Table 1 or Table 2 designated as a “down” gene in the gene directionality column of Table 1 or Table 2.

In embodiments, altering the gene signature comprises an activation of a network module designated in the network module column of Table 1 or Table 2.

In embodiments, the population of cells is derived from the subject, optionally wherein the population of cells is derived from the subject's bone marrow or the subject's blood, or the population of cells is not derived from the subject, or the population of cells is fractionated prior to contacting with at least one perturbagen, optionally wherein the fractionated cells are enriched for T cells.

In aspects and embodiments, there is provided a method for treating or preventing cancer, comprising administering to a subject in need thereof a therapeutically effective amount of at least one perturbagen selected from Table 3, or a variant thereof, wherein the at least one perturbagen is capable of changing a gene signature in a T cell.

In aspects and embodiments, there is provided a method for treating or preventing cancer, comprising administering to a subject in need thereof a population of cells, the population of cells having been contacted with at least one perturbagen selected from Table 4, or a variant thereof, wherein the at least one perturbagen is capable of changing a gene signature in a T cell.

In embodiments, the population of cells is derived from the subject, optionally wherein the population of cells is derived from the subject's bone marrow or the subject's blood, or the population of cells is not derived from the subject, or the population of cells is fractionated prior to contacting with at least one perturbagen, optionally wherein the fractionated cells are enriched for T cells.

In embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In embodiments, the cancer is a melanoma, optionally selected from metastatic melanoma, cutaneous melanoma, BRAF V600, Merkel cell carcinoma, cutaneous squamous cell carcinoma, a lung cancer, optionally selected from NSCLC, metastatic nonsquamous non-small cell lung cancer, metastatic small cell carcinoma, a head and neck cancer, optionally selected from head and neck squamous cell cancer, a bladder cancer, optionally selected from urothelial carcinoma, locally advanced or metastatic urothelial carcinoma, a breast cancer, optionally selected from triple-negative breast cancer), microsatellite Instability-high cancer, gastric cancer, optionally selected from gastric or gastroesophageal junction adenocarcinoma, metastatic colorectal cancer, cervical cancer, optionally selected from recurrent or metastatic cervical cancer, liver cancer, optionally selected from HCC, hematological disorder, optionally selected from primary mediastinal large B-cell lymphoma, and Hodgkin lymphoma.

In aspects and embodiments, there is provided a method for treating or preventing an infection, comprising administering to a subject in need thereof a therapeutically effective amount of at least one perturbagen selected from Table 3, or a variant thereof, wherein the at least one perturbagen is capable of changing a gene signature in a T cell. In aspects and embodiments, there is provided a method for treating or preventing an infection, comprising administering to a subject in need thereof a population of cells, the population of cells having been contacted with at least one perturbagen selected from Table 4, or a variant thereof, wherein the at least one perturbagen is capable of changing a gene signature in a T cell.

In embodiments, the population of cells is derived from the subject, optionally wherein the population of cells is derived from the subject's bone marrow or the subject's blood, or the population of cells is not derived from the subject, or the population of cells is fractionated prior to contacting with at least one perturbagen, optionally wherein the fractionated cells are enriched for T cells.

In embodiments, the infection is selected from bacterial infections, viral infections, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

In aspects and embodiments, there is provided a method of identifying a candidate perturbation for reducing or reversing the conversion of a T cell into an exhausted T cell, the method comprising, exposing the starting population of T cells to a perturbation, identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of T cells into exhausted T cells following exposure of the population of cells to the perturbation, and identifying the perturbation as a candidate perturbation for reducing or reversing the conversion of a T cell thereof based on the perturbation signature, wherein the perturbation signature is an increase in expression and/or activity in the T cell of one or more genes selected from Table 1 or Table 2 designated as an “up” gene in the gene directionality column of Table 1 or Table 2, and/or a decrease in expression and/or activity in the T cell of one or more genes selected from Table 1 or Table 2 designated as a “down” gene in the gene directionality column of Table 1 or Table 2.

In embodiments, altering the perturbation signature comprises an activation of a network module designated in the network module column of Table 1 or Table 2.

In embodiments, there is provided a method for making a therapeutic agent for a cancer or infection, comprising, (a) identifying a candidate perturbation according to a method disclosed herein, and (b) formulating the candidate perturbation as a therapeutic agent for the treatment of the disease or disorder.

In embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In embodiments, the infection is selected from bacterial infections, viral infections, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

In aspects and embodiments, there is provided a method for directing a change in cell state of a T cell in a subject, comprising: administering at least one perturbagen capable of altering a gene signature in the T cell to the subject, wherein altering the gene signature comprises an increase in expression and/or activity in the T cell of one or more genes selected from Table 1 designated as an “up” gene in the gene directionality column of Table 1 and/or a decrease in expression and/or activity in the T cell of one or more genes selected from Table 1 designated as a “down” gene in the gene directionality column of Table 1; wherein the change in cell state is a prevention or reduction of a cellular transition to an exhausted T cell state; and wherein the T cell is selected from an effector T cell, an exhausted T cell, and a naïve T cell.

In embodiments, the change in cell state is a prevention or reduction of a transition of an effector T cell to an exhausted T cell state.

In embodiments, the at least one perturbagen is selected from Table 3, or a variant thereof.

In embodiments, the at least one perturbagen selected from Table 3 comprises at least 2, at least 3, at least 4, or at least 5, at least 6, at least 7, at least 8, at least 9, at least 10, at least 11, at least 12, at least 13, at least 14, at least 15, at least 16, at least 17, at least 18, at least 19, and at least 20 perturbagens selected from Table 3, or variants thereof.

In embodiments, the T cells are CD8+ T cells.

In embodiments, the one or more genes selected from Table 1 comprises at least one of NFKBIA, TSC22D3, ZFP36, ARHGEF2, FHL2, STMN1, CDC25B, CCND3, TMEM109, E2F2, SCP2, PDLIM1, CORO1A, ATP11B, SATB1, CXCR4, ARL4C, CTSD, CD44, ZMIZ1, TBXA2R, GNA15, PRKCQ, RHOA, SLC25A4, PRUNE, CDC42, TIMP2, FAM69A, NRAS, BHLHE40, DNAJC15, GNAI2, DHRS7, CYTH1, ADGRE5, IGF2R, ADRB2, EIF4EBP1, FAS, MRPS16, TMEM50A, S100A4, RSU1, SPTAN1, S100A13, RAC2, REEP5, MACF1, PLP2, and TWF2.

In embodiments, the one or more genes selected from Table 1 comprises at least one of STAT1, DUSP6, INPP1, PSMB8, MLEC, ID2, RGS2, UBE2L6, SSBP2, PRKCH, ALDOA, ADGRG1, MFSD10, HERC6, CEP57, FBXL12, ICAM1, GLRX, PSME2, MYCBP2, IKZF1, PSMB10, PSME1, EVL, MBNL1, FYN, DNAJB6, FOXO3, TSPAN3, SYNE2, and RPS6.

In aspects and embodiments, there is provided an ex-vivo method for directing a change in cell state of a T cell, comprising: contacting a population of cells with at least one perturbagen capable of altering a gene signature in the T cell, wherein altering the gene signature comprises an increase in expression and/or activity in the T cell of one or more genes selected from Table 2 designated as an “up” gene in the gene directionality column of Table 2 and/or a decrease in expression and/or activity in the T cell of one or more genes selected from Table 2 designated as a “down” gene in the gene directionality column of Table 2; wherein the change in cell state is a prevention or reduction of a cellular transition to an exhausted T cell state; and wherein the T cell is selected from an effector T cell, an exhausted T cell, and a naïve T cell.

In embodiments, the change in cell state is a prevention or reduction of a transition of an effector T cell to an exhausted T cell state.

In embodiments, the at least one perturbagen is selected from Table 4, or a variant thereof.

In embodiments, the at least one perturbagen selected from Table 4 comprises at least 2, at least 3, at least 4, or at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35, at least 36, at least 37, at least 38, at least 39, at least 40, at least 41, at least 42, or at least 43 perturbagens selected from Table 4, or variants thereof.

In embodiments, the T cells are CD8+ T cells.

In embodiments, the one or more genes selected from Table 2 comprises at least one of MYC, TES, CXCR4, IGFBP3, PRSS23, SYPL1, CYB561, CCNH, XBP1, RPS6, ADRB2, GDPD5, SORBS3, ZFP36, FOS, PXN, SLC25A4, DSG2, SATB1, IER3, SSBP2, RPS5, ATP1B1, and GADD45B.

In embodiments, the one or more genes selected from Table 2 comprises at least one of CDK6, MTHFD2, ID2, SCCPDH, SLC25A46, ETFB, HLA-DRA, CHN1, RAB27A, TBXA2R, NFKB2, ITGAE, SMC4, STMN1, GATA3, ETS1, IQGAP1, CAT, RALA, TSC22D3, CBLB, INPP4B, PLSCR1, NUSAP1, RGS2, EVL, PSMB8, HERPUD1, APBB2, MIF, SQSTM1, PGAM1, TWF2, DRAP1, ETV1, CCNA1, HTRA1, DUSP4, GAPDH, RPA3, ADGRG1, ACOT9, CALM3, SOX4, HMOX1, RHOA, S100A4, ANKRD10, FCHO1, KDM5B, SPTAN1, CTSD, HLA-DMA, FGFR4, SLC1A4, HSPB1, CDKN2A, STAT3, RAC2, TIAM1, RALGDS, and EZH2.

### BRIEF DESCRIPTION OF THE DRAWINGS

FIG. 1A a schematic of single cell manifold derived from data of all tumors, labelled with cell states. Macs, macrophages; B cell, B lymphocytes; T cell, T lymphocytes; NK, natural killer cells; Mal, malignant cells; CAF, cancer associated fibroblasts.

FIG. 1B illustrates the experimental scheme for MC38 syngeneic tumor model.

FIG. 2A shows manipulated cell states, in vivo decrease (if applicable). The figure shows that T cell exhaustion is blocked. The block in T cell exhaustion is revealed by a reduction in the expression of PD-1 and TIGIT on CD8+ T cells, in vivo. Perturbagens referenced refer to perturbagens of Table 3.

FIG. 2B shows manipulated cell states, in vivo decrease (if applicable). The figure shows an increase in the number of CD8+ T cells in vivo. Perturbagens referenced refer to perturbagens of Table 3.

FIG. 3A shows phenotypic change as a consequence of cell state change. The figure shows inhibition of tumor growth as a result of small molecule dosing. Perturbagens referenced refer to perturbagens of Table 3.

FIG. 3B shows phenotypic change as a consequence of cell state change. The figure shows reduced fold increase in tumor fold relative to the tumor at the start of dosing for those small molecule compound that affect tumor size. Perturbagens referenced refer to perturbagens of Table 3.

FIGS. 4A-4D show scRNAseq analysis for T cell exhaustion in the MC38 syngeneic mouse. Manifold for all cells captured in the experiment is shown in FIG. 4A. Using published cell-type gene expression signatures a distinct population of T cells was identified in the manifold (FIG. 4B). The positive control, pembrolizumab, enhanced the density of T cells (FIG. 4C (PBS) and 4D (pembrolizumab)).

FIGS. 5A-5B show an increased number of T cells (FIG. 5A) and natural killer cells associated with a decrease in tumor size (FIG. 5B). Perturbagens referenced refer to perturbagens of Table 3.

FIG. 6A shows memory and cytotoxic CD8+ T cells are elevated in responders to checkpoint inhibition, compared to non-responders who have elevated exhausted CD8+ T cells.

FIG. 6B shows Kaplan-Meier Curve in adenocarcinoma, stratified by CD8+ T cell exhaustion status (22 gene signature).

FIGS. 7A-7D show Compound A prevents T cell exhaustion in vitro. FIG. 7A shows experimental data demonstrating that Compound A restores T cell proliferation under exhausted conditions. FIG. 7B shows a graph of flow cytometry assessment of T cell numbers. FIG. 7C shows a graph of experimental data demonstrating that Compound A prevents expression of multiple immune checkpoint receptors (ICR). Expression of checkpoint receptors PD1, TIM3, LAG3 and TIGIT was assessed by flow cytometry. For each set of three bars, the left bar is expanded T cells, the middle bar is exhausted T cells, and the right bar is exhausted+Compound A. FIG. 7D shows a distribution map of the cell population demonstrating that transcriptionally, Compound A treated exhausted cells are indistinguishable from non-exhausted cells.

FIGS. 8A-8D show Compound A is immuno-modulatory via mild T cell receptor (TCR) inhibition. FIG. 8A shows a schematic of an immuno-modulation assay. FIG. 8B shows experimental data demonstrating the dose dependent immuno-modulatory effect of Compound A. The top curve represents immuno-modulation assay proliferation, the bottom curve represents immuno-modulation assay cell number. FIG. 8C shows a schematic of TCR inhibition assay.

FIG. 8D shows experimental data demonstrating that Compound A is a mild TCR inhibitor. Flow cytometry of Compound A was assessed at 300 nM partially inhibited early CD69 expression following TCR activation.

FIGS. 9A-9M show Compound A reduces T cell exhaustion in vivo but with limited window of activity. FIG. 9A shows a non-limiting example of workflow of Compound A evaluation in a CT26 model. FIGS. 9B-9F show experimental data demonstrating that Compound A treatment at low dose recapitulates anti-PD1 mediated population changes while high dose treatment induces accumulation of naïve cells. FIG. 9B shows the analysis of CD8 T cells population changes following 7 days treatment with Compound A by scRNAseq and flow cytometry. FIGS. 9C-9F show Leiden Clustering of CD8 T cell populations normalized to vehicle and proportion of CD8 T cell populations analyzed by flow cytometry (progenitor exhausted TCF7+TOX+ (FIG. 9C), exhausted TCF7−TOX+ (FIG. 9D), activated TCF7−TOX− (FIG. 9E), naïve/memory TCF7+TOX− (FIG. 9F)). FIG. 9G shows experimental data demonstrating that Compound A treatment reduces TOX expression in exhausted cells. TOX expression on exhausted cells by scRNAseq and flow cytometry in CT26 model was quantified. FIG. 9H shows a non-limiting example of workflow of Compound A evaluation in MC38 model. FIGS. 9I-9M show experimental data demonstrating that Compound A pulsed treatment at high dose recapitulates anti-PD1 activity and reduces TOX expression on Exhausted cells in most animals. The graphs show a proportion of CD8 T cells populations and TOX expression on exhausted T cells analyzed by flow cytometry. (progenitor exhausted TCF7+TOX+ (FIG. 9I), exhausted TCF7−TOX+ (FIG. 9J), activated TCF7−TOX−(FIG. 9K), naïve/memory TCF7+TOX−(FIG. 9L)). FIG. 9M shows quantification of TOX expression on exhausted cells by flow cytometry in MC38 model. One way ANOVA followed by posthoc Dunnett's Vs Vehicle *p<0.05, ** p<0.01, *** p<0.001, *** p<0.0001, **** p<0.00001 (N=5 mice per group).

FIG. 10 is a schematic of experimental setup for a screen of compounds in a CD8+ T cell exhaustion assay. On day 7, the cell number was determined by CellTiter-Glo assay (CTG).

FIG. 11 shows the effect of small molecules on CD8+ T cell number in a 7-day exhaustion assay. Cell number was determined using CellTiter-Glo and data are represented as fold change in CD8+ T cell number relative to the DMSO control at day 7. Perturbagens referenced refer to perturbagens of Table 4.

FIGS. 12A-12C show hit compounds from predictions made to prevent effector T cells transitioning into exhausted T cells. Compounds were incubated with CD8+ T cells during the ex vivo exhaustion assay (6× anti-CD3/CD28). Mean±standard deviation shown; square symbol, DMSO control.

FIG. 13A shows single cell manifold of exhausted (dark gray) and non-exhausted (light gray) T cells. FIGS. 13B-13F show the expression levels of different markers of T cell exhaustion plotted over the complete manifold for T cells (on a grayscale with darker gray showing higher expression level). Using scRTNAseq, we show that specific markers are associated with the exhausted T-cell state, including PD-1 (FIG. 13B), CTLA-4 (FIG. 13C), LAG-3 (FIG. 13D), TIM-3 (FIG. 13E), TIGIT (FIG. 13F).

### DETAILED DESCRIPTION

The present disclosure is based, in part, on the discovery that T cells, including those in various states of activation and/or functionality can be characterized by specific gene signatures. Additionally, the present disclosure is based on the discovery that certain active agents (i.e., perturbagens) can alter these specific gene signatures. Such alteration is associated with the acquisition of specific cell states by the T cells. For instance, present disclosure is based, in part, on the discovery of compounds and methods that shift populations of T cells away from an exhausted state. These perturbagens are, in some instances, useful as therapeutics and/or in directing a change in the cell state of T cells and provide benefit by, e.g., by preventing or reducing T cell transition to an exhausted T cell state; preventing or reducing transition of an effector T cell to an exhausted T cell state; preventing or reducing transition of a naïve T cell to an exhausted T cell state; stimulating or increasing transition of an exhausted T cell to an effector T cell; stimulating or increasing transition of a naïve T cell to an effector T cell; stimulating or increasing cell death of an exhausted T cell; preventing or reducing cell death of an effector T cell and/or naïve T cell; and/or increasing the number of one or more of effector T cells and naïve T cells.

### In-Vivo Methods

### Genes Signatures

Cell state transitions (i.e., a transition in a cell's state from a first cell state to a second cell state) are characterized by a change in expression of genes in the cell. Changes in gene expression may be quantified as, e.g., an increase in mRNA expressed for a specific gene or a decrease in mRNA expressed for another specific gene; especially significant here may be mRNAs that encode transcription factors. Collectively, the sum of multiple differences in gene expression between one cell type or cells of one state relative to another cell type or cells of another state are referred to herein as a gene signature. For instance, a gene signature, in various embodiments, includes the sum of multiple differences in gene expression between non-exhausted and exhausted T cells.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may be useful to first characterize the cell type or cells of a specific state by surface proteins that are characteristic of the cell type or cells of a specific state.

Knowing the gene signature for each cell type or cells of a specific state provides insight into what genes impact or are associated with the process of transition to other cell types.

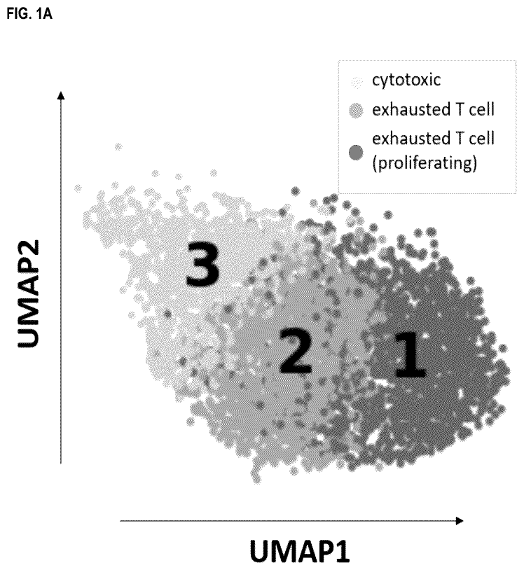

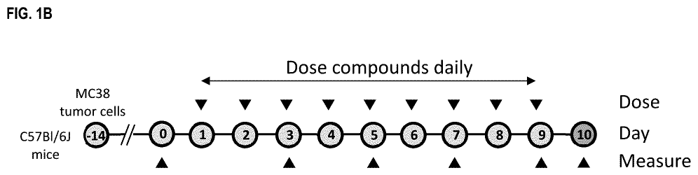

FIG. 1A, shows annotated clusters that associate gene signature with cell types or cells of an activation state. Clusters 1 and 2 show exhausted T cells, cluster 3 shows cytotoxic T cells and cluster 9 shows memory cells.

Genes that are differentially expressed and positively associated with the promotion of non-exhausted T cells are listed in Table 1. The genes listed in Table 1 show an increase or decrease in expression and/or activity in the cell state change.

TABLE 1

| | | Gene | Gene | Network |
|---|---|---|---|---|
| | Gene | Entrez_ID | Directionality | Module |
| 0 | NFKBIA | 4792 | up | 0 |
| 1 | TSC22D3 | 1831 | up | 0 |
| 2 | ZFP36 | 7538 | up | 0 |
| 3 | ARHGEF2 | 9181 | up | 0 |
| 4 | FHL2 | 2274 | up | 0 |
| 5 | STAT1 | 6772 | down | 0 |
| 6 | DUSP6 | 1848 | down | 0 |
| 7 | INPP1 | 3628 | down | 0 |
| 8 | STMN1 | 3925 | up | 1 |
| 9 | CDC25B | 994 | up | 1 |
| 10 | CCND3 | 896 | up | 1 |
| 11 | TMEM109 | 79073 | up | 1 |
| 12 | E2F2 | 1870 | up | 1 |
| 13 | SCP2 | 10388 | up | 1 |
| 14 | PSMB8 | 5696 | down | 1 |
| 15 | MLEC | 9761 | down | 1 |
| 16 | PDLIM1 | 9124 | up | 2 |
| 17 | CORO1A | 11151 | up | 2 |
| 18 | ATP11B | 23200 | up | 2 |
| 19 | SATB1 | 6304 | up | 2 |
| 20 | CXCR4 | 7852 | up | 2 |
| 21 | ID2 | 3398 | down | 2 |
| 22 | RGS2 | 5997 | down | 2 |
| 23 | ARL4C | 10123 | up | 3 |
| 24 | CTSD | 1509 | up | 3 |
| 25 | CD44 | 960 | up | 3 |
| 26 | ZMIZ1 | 57178 | up | 3 |
| 27 | TBXA2R | 6915 | up | 3 |
| 28 | UBE2L6 | 9246 | down | 3 |
| 29 | SSBP2 | 23635 | down | 3 |
| 30 | GNA15 | 2769 | up | 4 |
| 31 | PRKCQ | 5588 | up | 4 |
| 32 | RHOA | 387 | up | 4 |
| 33 | SLC25A4 | 291 | up | 4 |
| 34 | PRUNE | 58497 | up | 4 |
| 35 | PRKCH | 5583 | down | 4 |
| 36 | ALDOA | 226 | down | 4 |
| 37 | CDC42 | 998 | up | 5 |
| 38 | TIMP2 | 7077 | up | 5 |
| 39 | FAM69A | 388650 | up | 5 |
| 40 | NRAS | 4893 | up | 5 |
| 41 | ADGRG1 | 9289 | down | 5 |
| 42 | MFSD10 | 10227 | down | 5 |
| 43 | BHLHE40 | 8553 | up | 6 |
| 44 | DNAJC15 | 29103 | up | 6 |
| 45 | HERC6 | 55008 | down | 6 |
| 46 | CEP57 | 9702 | down | 6 |
| 47 | FBXL12 | 54850 | down | 6 |
| 48 | ICAM1 | 3383 | down | 6 |
| 49 | GNAI2 | 2771 | up | 7 |
| 50 | DHRS7 | 51635 | up | 7 |
| 51 | CYTH1 | 9267 | up | 7 |
| 52 | ADGRE5 | | up | 7 |
| 53 | IGF2R | 3482 | up | 7 |
| 54 | GLRX | 2745 | down | 7 |
| 55 | ADRB2 | 154 | up | 8 |
| 56 | EIF4EBP1 | 1978 | up | 8 |
| 57 | PSME2 | 5721 | down | 8 |
| 58 | MYCBP2 | 23077 | down | 8 |
| 59 | IKZF1 | 10320 | down | 8 |
| 60 | FAS | 355 | up | 9 |
| 61 | MRPS16 | 51021 | up | 9 |
| 62 | PSMB10 | 5699 | down | 9 |
| 63 | PSME1 | 5720 | down | 9 |
| 64 | EVL | 51466 | down | 9 |
| 65 | TMEM50A | 23585 | up | 10 |
| 66 | MBNL1 | 4154 | down | 10 |
| 67 | FYN | 2534 | down | 10 |
| 68 | DNAJB6 | 10049 | down | 10 |
| 69 | FOX03 | 2309 | down | 10 |
| 70 | S100A4 | 6275 | up | 11 |
| 71 | RSU1 | 6251 | up | 11 |
| 72 | SPTAN1 | 6709 | up | 11 |
| 73 | TSPAN3 | 10099 | down | 11 |
| 74 | S100A13 | 6284 | up | 12 |
| 75 | RAC2 | 5880 | up | 12 |
| 76 | SYNE2 | 23224 | down | 12 |
| 77 | RPS6 | 6194 | down | 12 |
| 78 | REEP5 | 7905 | up | 13 |
| 79 | MACF1 | 23499 | up | 13 |
| 80 | PLP2 | 5355 | up | 13 |
| 81 | TWF2 | 11344 | up | 13 |

In Table 1 and associated embodiments:
“Gene ID”: at the time of filing the present disclosure, the World Wide Web at ncbi.nlm.nih.gov/gene provides a description of and the nucleic acid sequence for each GeneID listed in Table 1; the contents of each of which is incorporated herein by reference in its entirety.
“Up” indicates a gene for which an increase in expression and/or activity in the T cell is associated with the gene signature.
“Down” indicates a gene for which a decrease in expression and/or activity in the T cell is associated with the gene signature.
A “network module” (sometimes also referred to as “module”) is a set of genes whose activity and/or expression are mutually predictive and, individually and collectively, are correlated with regard to a cell state change, which correlation may be positive or negative. That is, a module may contain genes that are positively associated with the cell state transition-such that an increase in expression and/or activity of the gene associated with the cell state transition; as well as genes that are negatively associated with the cell state transition such that a decrease in expression and/or activity of the gene associated with the cell state transition.

In certain embodiments, a network module includes genes in addition (or substituted for) to those exemplified in Table 1, which should be viewed as illustrative and not limiting unless expressly provided, namely with genes with correlated expression. A correlation, e.g., by the method of Pearson or Spearman, is calculated between a query gene expression profile for the desired cell state transition and one or more of the exemplary genes recited in the module. Those genes with a correlation with one or more genes of the module of at significance level below p=0.05 (e.g., 0.04, 0.03, 0.02, 0.01, 0.005, 0.001, 0.0005, 0.0001, or less) can be added to, or substituted for, other genes in the module.

“Activation of a network module” refers to a perturbation that modulates expression and/or activity of 2 or more genes (e.g., 3, 4, 5, 6 . . . genes; or about 10, 20, 25, 30, 40, 50, 60, 70, 75, 80, 85, 90, 95, or 100%) within a module, which modulation may be an increase or decrease in expression and/or activity of the gene as consonant with the modules described in Table 1. In certain embodiments, a perturbation activates multiple network modules for the desired cell state transition, such as 2, 3, 4, 5, 6, 7, 8, 9, 10, 11, 12, or all 13 modules.

In some embodiments, one or more genes of network module 0 are modulated. In some embodiments, the present disclosure relates to the activation of network module 0, e.g., one or more of (inclusive of all of) NFKBIA, TSC22D3, ZFP36, ARHGEF2, FHL2, STAT1, DUSP6, and INPP1.

In some embodiments, one or more genes of network module 1 are modulated. In some embodiments, the present disclosure relates to the activation of network module 1, e.g., one or more of (inclusive of all of) STMN1, CDC25B, CCND3, TMEM109, E2F2, SCP2, PSMB8, and MLEC.

In some embodiments, one or more genes of network module 2 are modulated. In some embodiments, the present disclosure relates to the activation of network module 2, e.g., one or more of (inclusive of all of) PDLIM1, CORO1A, ATP11B, SATB1, CXCR4, ID2, and RGS2.

In some embodiments, one or more genes of network module 3 are modulated. In some embodiments, the presents relate to the activation of network module 3, e.g., one or more of (inclusive of all of) ARL4C, CTSD, CD44, ZMIZ1, TBXA2R, UBE2L6, and SSBP2.

In some embodiments, one or more genes of network module 4 are modulated. In some embodiments, the presents relate to the activation of network module 4, e.g., one or more of (inclusive of all of) GNA15, PRKCQ, RHOA, SLC25A4, PRUNE, PRKCH, and ALDOA.

In some embodiments, one or more genes of network module 5 are modulated. In some embodiments, the presents relate to the activation of network module 5, e.g., one or more of (inclusive of all of) CDC42, TIMP2, FAM69A, NRAS, ADGRG1, and MFSD10.

In some embodiments, one or more genes of network module 6 are modulated. In some embodiments, the presents relate to the activation of network module 6, e.g., one or more of (inclusive of all of) BHLHE40, DNAJC15, HERC6, CEP57, FBXL 12, and ICAM1.

In some embodiments, one or more genes of network module 7 are modulated. In some embodiments, the presents relate to the activation of network module 7, e.g., one or more of (inclusive of all of) GNAI2, DHRS7, CYTH1, ADGRE5, IGF2R, and GLRX.

In some embodiments, one or more genes of network module 8 are modulated. In some embodiments, the presents relate to the activation of network module 8, e.g., one or more of (inclusive of all of) ADRB2, EIF4EBP1, PSME2, MYCBP2, and IKZF1.

In some embodiments, one or more genes of network module 9 are modulated. In some embodiments, the presents relate to the activation of network module 9, e.g., one or more of (inclusive of all of) FAS, MRPS16, PSMB10, PSME1, and EVL.

In some embodiments, one or more genes of network module 10 are modulated. In some embodiments, the presents relate to the activation of network module 10, e.g., one or more of (inclusive of all of) TMEM50A, MBNL1, FYN, DNAJB6, and FOXO3.

In some embodiments, one or more genes of network module 11 are modulated. In some embodiments, the presents relate to the activation of network module 11, e.g., one or more of (inclusive of all of) S100A4, RSU1, SPTAN1, and TSPAN3.

In some embodiments, one or more genes of network module 12 are modulated. In some embodiments, the presents relate to the activation of network module 12, e.g., one or more of (inclusive of all of) S100A13, RAC2, SYNE2, and RPS6.

In some embodiments, one or more genes of network module 13 are modulated. In some embodiments, the presents relate to the activation of network module 13, e.g., one or more of (inclusive of all of) REEP5, MACF1, PLP2, and TWF2.

In some embodiments, the present methods alter a gene signature in the sample of cells, comprising an activation of a network module designated in the network module column of Table 1.

In some embodiments, the activation of the network module designated in the network module column of Table 1 comprises modulating expression and/or activity of 2 or more genes within a network module.

In some embodiments, the activation of the network module designated in the network module column of Table 1 comprises modulating expression and/or activity of all of the genes within a network module.

In some embodiments, the activation of the network module designated in the network module column of Table 1 comprises modulating expression and/or activity of 2 or more genes within 2 or more network modules. In some embodiments, the activation of the network module designated in the network module column of Table 1 comprises modulating expression and/or activity of 2 or more genes (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, or 50 or more genes) within 2 or more network modules (e.g. 2 or more, or 3 or more, or 4 or more, or 5 or more, or 6 or more, or 7 or more, or 8 or more, or 9 or more, or 10 or more, or 11 or more, or 12 or more, or 13 or more network modules).

### Perturbagens

A perturbagen useful in the present disclosure can be a small molecule, a biologic, a protein, a nucleic acid, such as a cDNA over-expressing a wild-type gene or an mRNA encoding a wild-type gene, or any combination of any of the foregoing. Illustrative perturbagens useful in the present disclosure are listed in Table 3.

TABLE 3
Exemplary Perturbagens

| | | Molecular | | |
|---|---|---|---|---|
| | | weight | | |
| Perturbagen No. | Molecular formula | (g/mol) | Dose | |
| 1 | C₂₂H₁₉ClN₄O₅ | 454.9 | 10 | mg/kg |
| 2 | C₁₆H₁₂N₂O₃ | 280.28 | 5 | mg/kg |
| 3 | C₂₁H₂₃N₃O₂ | 349.4 | 30 | mg/kg |
| 4 | C₁₅H₈ClF₆NO₂ | 383.67 | 30 | mg/kg |
| 5 | C₁₉H₁₆N₄O₃ | 348.4 | 3 | mg/kg |
| 6 | C₁₅H₁₄O₅ | 274.27 | 30 | mg/kg |
| 7 | C₁₈H₁₉Cl₂NO₄ | 384.2 | 3 | mg/kg |
| 8 | C₁₁H₁₃NO₅ | 239.22 | 10 | mg/kg |
| 9 | C₂₃H₁₈ClF₂N₃O₃S | 489.9 | 3 | mg/kg |
| 10 | C₃₀H₂₃N₅O | 469.5 | | |
| 11 | C₂₇H₃₃N₅O₅S | 539.6 | 10 | mg/kg |
| 12 | C₂₁H₂₀N₄ | 328.4 | 3 | mg/kg |
| 13 | C₈H₁₇NO₅ | 207.22 | 30 | mg/kg |
| 14 | C₂₃H₂₃N₃O₃ | 389.4 | 10 | mg/kg |
| 15 | C₂₈H₂₃N₉O | 501.5 | 10 | mg/kg |
| 16 | C₁₉H₁₈ClF₃N₂O₅S | 478.9 | 30 | mg/kg |
| 17 | C₁₈H₁₆O₈ | 360.3 | 30 | mg/kg |
| 18 | C₂₄H₂₁ClF₃NO₄S | 511.9 | 30 | mg/kg |
| 19 | C₇₅H₇₀N₆O₆ | 1151.4 | | |
| 20 | C₃₀H₄₂O₇ | 514.6 | | |
| 21 | C₂₄H₂₀Cl₂FN₅O₂ | 500.35 | | |

In various embodiments herein, a perturbagen of Table 3 encompasses the perturbagens named and/or other perturbagens identified in Table 3. Thus, the named perturbagens of Table 3 represent examples of perturbagens of the present disclosure.

In Table 3, the column titled dose is the concentration of a perturbagen that is capable of increasing gene expression in a T cell, as assayed, at least, by single cell gene expression profiling (GEP).

In embodiments, a perturbagen used in the present disclosure is a variant of a perturbagen of Table 3. A variant may be a derivative, analog, enantiomer or a mixture of enantiomers thereof or a pharmaceutically acceptable salt, solvate, hydrate, co-crystal, clathrate, or polymorph of the perturbagen of Table 3. A variant of a perturbagen of Table 3 retains the biological activity of the perturbagen of Table 3.

### Methods and Perturbagens for Directing a Change in Cell State

In some aspects, the present disclosure is related to a method for directing a change in cell state of a T cell in a subject, comprising administering at least one perturbagen selected from Table 3, or a variant thereof to the subject, the perturbagen being capable of altering a gene signature in the T cell.

Another aspect of the present disclosure is related to a method for directing a change in cell state of a T cell in a subject, comprising administering at least one perturbagen capable of altering a gene signature in the T cell to the subject, wherein altering the gene signature comprises an increase in expression and/or activity in the T cell of one or more genes selected from Table 1 designated as an “up” gene in the gene directionality column of Table 1 and/or a decrease in expression and/or activity in the T cell of one or more genes selected from Table 1 designated as a “down” gene in the gene directionality column of Table 1. In some embodiments, the method is related altering the gene signature which includes an increase in expression and/or activity in the T cell of one or more genes selected from Table 1 designated as an “up” gene in the gene directionality column of Table 1. In some embodiments, altering the gene signature includes a decrease in expression and/or activity in the T cell of one or more genes selected from Table 1 designated as a “down” gene in the gene directionality column of Table 1.

In one aspect, the present disclosure is related to a method for directing a change in cell state of a T cell in a subject. This method includes the following steps i) administering at least one perturbagen selected from Table 3, or a variant thereof to the subject, the perturbagen being capable of altering a gene signature in the T cell, wherein altering the gene signature comprises an increase in expression and/or activity in the T cell of one or more genes selected from Table 1 designated as a “up” gene in the gene directionality column of Table 1 and/or a decrease in expression and/or activity in the T cell of one or more genes selected from Table 1 designated as a “down” gene in the gene directionality column of Table 1. In some embodiments, the method is related to altering the gene signature which includes an increase in expression and/or activity in the T cell of one or more genes selected from Table 1 designated as an “up” gene in the gene directionality column of Table 1. In some embodiments, altering the gene signature includes a decrease in expression and/or activity in the T cell of one or more genes selected from Table 1 designated as an “up” gene in the gene directionality column of Table 1.

In some embodiments, the T cell is selected from an effector T cell, exhausted T cell, and naïve T cell. In some embodiments, the T cells are CD8+ T cells. In other embodiments, the T cells are CD4+ T cells. In some embodiments, in the methods described herein, the change in cell state is within the tumor microenvironment, if the subject is afflicted with cancer. In some embodiments, the change in cell state is within the thymus. In other embodiments, the change in cell state is within the bone marrow.

In some embodiments, the methods described herein are related to a change in cell state where the change is the cell state is selected from:
i) prevention or reduction of a cellular transition to an exhausted T cell state;
ii) prevention or reduction of a transition of an effector T cell to an exhausted T cell state;
iii) prevention or reduction of a transition of a naïve T cell to an exhausted T cell state;
iv) stimulation or increase of a transition of an exhausted T cell to an effector T cell;
v) stimulation or increase of a transition of a naïve T cell to an effector T cell;
vi) stimulation or increase of a cell death of an exhausted T cell;
vii) prevention or reduction of a cell death of an effector T cell and/or a naïve T cell; and
viii) increase in the number of one or more of effector T cells and naïve T cells.

In some embodiments, the change in cell state provides an increase in the number of one or more of effector T cells and naïve T cells relative to the number of effector T cells or naïve T cells obtained from a population of T cells that is not contacted with the at least one perturbagen. In other embodiments, the change in cell state provides an increase in the number of one or more of effector T cells and naïve T cells relative to the number of effector T cells or naïve T cells obtained from a population of T cells prior to contacting with the at least one perturbagen.

In some embodiments, in the methods described herein, the change in cell state provides a decrease in the number of one or more of exhausted T cells. In embodiments, the change in cell state provides a decrease in the number of one or more of exhausted T cells relative to the number of exhausted T cells obtained from a population of T cells that is not contacted with the at least one perturbagen. In some embodiments, the change in cell state provides a decrease in the number of one or more of exhausted T cells relative to the number of exhausted T cells obtained from a population of T cells prior to contacting with the at least one perturbagen.

In embodiments, the methods described herein are such that the ratio of the number of effector T cells and/or naïve T cells to the number of exhausted T cells is increased relative to the ratio obtained from a population of T cells that is not contacted with the at least one perturbagen. In other embodiments, the ratio of the number of effector T cells and/or naïve T cells to the number of exhausted T cells is increased relative to the ratio obtained from a population of T cells prior to contacting with the at least one perturbagen.

In some embodiments, the increase in the number of effector T cells and/or naïve T cells is due in part to:
i) increased cell proliferation of the effector T cells and/or naïve T cells;
ii) increased lifespan of the effector T cells and/or naïve T cells; and
iii) reduced cell death among the effector T cells and/or naïve T cells.

In some embodiments, the decrease in the number of exhausted T cells is due in part to decreased cell proliferation of the T cells. In embodiments, the decrease in the number of exhausted T cells is due in part to a decreased lifespan of the T cells. In some embodiments, the decrease in the number of exhausted T cells is due in part to increased cell death among the T cells.

In some embodiments, the number of effector T cells and/or naïve T cells is increased after contacting the population of T cells with the at least one perturbagen. In other embodiments, the number of exhausted T cells is decreased after contacting the population of T cells with the at least one perturbagen.

In some embodiments, in the methods described herein, the ratio of the number of effector T cells and/or naïve T cells to the number of exhausted T cells is increased relative to the ratio obtained from a population of T cells that is not contacted with the at least one perturbagen. In other embodiments, in the methods described herein, the ratio of the number of effector T cells and/or naïve T cells to the number of exhausted T cells is increased relative to the ratio in the population of T cells prior to contacting with the at least one perturbagen.

In some embodiments, the methods described herein promote the formation of a T cells that are responsive to antigen.

In embodiments, the antigen is presented by a professional antigen-presenting cell.

In some embodiments, the methods described herein promote the formation of T cells that are responsive to both TCR engagement and co-stimulation. In other embodiments, the methods described herein promote the formation of T cells that are capable of elaborating cytokines. The cytokines can be, e.g., IL-2, IFNγ, TNFα, and a CC chemokine (or β-chemokine).

In some embodiments, the methods described herein promote the formation of T cells that are capable of proliferating, e.g. in the presence of TCR engagement and co-stimulation. In embodiments, the methods described herein promote the formation of T cells that are anabolic. In other embodiments, the methods described herein promote the formation of T cells that are cytotoxic. In embodiments, the methods described herein promote the formation of effector T cells.

In some embodiments, the methods described herein repress the formation of a T cells that are not responsive or poorly responsive to antigen. In embodiments, the antigen is presented by a professional antigen-presenting cell.

In some embodiments, the methods described herein repress the formation of T cells that are not responsive or poorly responsive to both TCR engagement and co-stimulation. In other embodiments, the methods described herein repress the formation of T cells that are incapable or poorly capable of elaborating cytokines. In embodiments, such cytokines are selected from IL-2, IFNγ, TNFα, and a CC chemokine (or β-chemokine).

In some embodiments, the methods described herein repress the formation of T cells that are incapable or poorly capable of proliferating, e.g. in the presence of signal 1 and signal 2. In order to activate a cytotoxic or helper T cell to proliferate and differentiate into an effector cell, an antigen-presenting cell provides two kinds of signals: signal 1 and signal 2. In some embodiments, signal 1 is provided by a foreign peptide bound to an MHC protein on the surface of the presenting cell. This peptide-MHC complex signals through the T cell receptor and its associated proteins. In some embodiments, signal 2 is provided by costimulatory proteins, e.g., the B7 proteins (CD80 and CD86), which are recognized by the co-receptor protein CD28 on the surface of the T cell. The expression of B7 proteins on an antigen-presenting cell is, e.g., induced by pathogens during the innate response to an infection. Effector T cells act to promote the expression of B7 proteins on antigen-presenting cells, creating a positive feedback loop that amplifies the T cell response. The combined actions of signal 1 and signal 2 stimulate the T cell to proliferate and begin to differentiate into an effector cell.

In some embodiments, the methods described herein repress the formation of T cells that are not anabolic or minimally anabolic. In other embodiments, the methods described herein repress the formation of T cells that are not cytotoxic or minimally cytotoxic. In embodiments, the methods repress the formation of exhausted T cells.

In some embodiments, the methods described herein reduce the number of T cells which demonstrate expression or increased expression of one of more of PD-1, PD-L1, PD-L2, CTLA-4, LAG-3, TIM-3, 2B4/CD244/SLAMF4, CD160, and TIGIT. In embodiments, the methods described herein prevent or reduce formation of T cells which demonstrate expression or increased expression of one of more of PD-1, PD-L1, PD-L2, CTLA-4, LAG-3, TIM-3, 2B4/CD244/SLAMF4, CD160, and TIGIT.

In some embodiments, the exhausted T cells according to the present disclosure demonstrate expression or increased expression of one of more of PD-1, PD-L1, PD-L2, CTLA-4, LAG-3, TIM-3, 2B4/CD244/SLAMF4, CD160, and TIGIT. In some embodiments, the exhausted T cells demonstrate expression or increased expression of a transcription factor selected from TOX, TCF1, NR4A and NFAT. In embodiments, the exhausted T cells demonstrate a loss or reduction of IL-2 productive capacity, proliferative capacity, and/or cytolytic activity. In embodiments, the exhausted T cells demonstrate a loss or reduction of TNFα, IFNγ, and CC chemokine (or β-chemokine) signaling.

In some embodiments, the exhausted T cells demonstrate degranulation and/or increased or high expression of granzyme B. In other embodiments, the exhausted T cells demonstrate poor responsiveness to IL-7 and/or IL-15.

In some embodiments, the methods described herein include administering IL-2.

In some embodiments, the methods described herein include administering at least one perturbagen selected from Table 3, or a variant thereof. In some embodiments, the method includes administering at least 2, at least 3, at least 4, or at least 5, at least 6, at least 7, at least 8, at least 9, at least 10, at least 11, at least 12, at least 13, at least 14, at least 15, at least 16, at least 17, at least 18, at least 19 or at least 20 perturbagens selected from Table 3, or variants thereof.

In some embodiments, in the methods described herein, the one or more genes are selected from Table 1 designated as an “up” gene in the gene directionality column of Table 1. In some embodiments, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, and 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more genes are selected from Table 1. In some embodiments, the one or more genes selected from Table 1 include at least one of NFKBIA, TSC22D3, ZFP36, ARHGEF2, FHL2, STMN1, CDC25B, CCND3, TMEM109, E2F2, SCP2, PDLIM1, CORO1A, ATP11B, SATB1, CXCR4, ARL4C, CTSD, CD44, ZMIZ1, TBXA2R, GNA15, PRKCQ, RHOA, SLC25A4, PRUNE, CDC42, TIMP2, FAM69A, NRAS, BHLHE40, DNAJC15, GNAI2, DHRS7, CYTH1, ADGRE5, IGF2R, ADRB2, EIF4EBP1, FAS, MRPS16, TMEM50A, S100A4, RSU1, SPTAN1, S100A13, RAC2, REEP5, MACF1, PLP2, and TWF2.

In some embodiments, in the methods described herein, the one or more genes are selected from Table 1 designated as a “down” gene in the gene directionality column of Table 1. In some embodiments, one or more genes selected from Table 1 include 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, and 20 or more, 21 or more, 22 or more, 23 or more, 24 or more, 25 or more, 26 or more, 27 or more, 28 or more, 29 or more, 30 or more, or 31 or more genes selected from Table 1. In some embodiments, the one or more genes selected from Table 1 comprises at least one of STAT1, DUSP6, INPP1, PSMB8, MLEC, ID2, RGS2, UBE2L6, SSBP2, PRKCH, ALDOA, ADGRG1, MFSD10, HERC6, CEP57, FBXL12, ICAM1, GLRX, PSME2, MYCBP2, IKZF1, PSMB10, PSME1, EVL, MBNL1, FYN, DNAJB6, FOXO3, TSPAN3, SYNE2, and RPS6. Methods for determining the extension of the lifespan of a specific cell type or a reduction of cell death is well known in the art. As examples, markers for dying cells, e.g., caspases can be detected, or dyes for dead cells, e.g., methylene blue, may be used. Methods for counting cells are well known in the art. Non-limiting examples include hemocytometry, flow cytometry, and cell sorting techniques, e.g., fluorescence activated cell sorting (FACS).

In embodiments, an increase in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (including DMSO).

In various embodiments, an increase in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (including DMSO).

In embodiments, contacting the population of cells comprising a T cell (including, e.g., naïve T cell, effector T cell and exhausted T cell) occurs in a subject. In embodiments, the subject is a human. In embodiments, the human is an adult human.

In yet another aspect, the present disclosure provides a perturbagen for use in any herein disclosed method. In a further aspect, the present disclosure provides a pharmaceutical composition comprising a perturbagen for use in any herein disclosed method.

Embodiments associated with the above aspects are likewise relevant to the present aspect. In other words, each of the embodiments mentioned above for the above aspects may be revised/adapted to be applicable to the present aspect.

### Methods and Perturbagens for Treating a Disease or Disorder

The ability of a perturbagen to mitigate or stop T cell exhaustion would be valuable in designing a therapeutic composition for the treatment of a disease. As an example, for a disease characterized by an increased number of exhausted T cells, a therapeutic composition comprising a perturbagen that decreases the number of exhausted T cells could be beneficial. Similarly, for a disease characterized by an increased number of exhausted T cells, a therapeutic composition comprising a perturbagen that i) increases expression and/or activity in the T cell, ii) prevents or reduces transition of an effector T cell to an exhausted T cell, iii) prevents or reduces transition of a naïve T cell to an exhausted T cell, iv) stimulates or increases transition of an exhausted T cell to an effector T cell, v) stimulates or increases transition of a naïve T cell to an effector T cell, vi) stimulates or increases cell death of an exhausted T cell, vii) prevents or reduces cell death of an effector T cell and/or a naïve T cell, viii) provides an increase in the number of one or more of effector T cells and naïve T cells can be beneficial.

In one aspect, the present disclosure is related to a method for treating a disease or disorder characterized by insufficient T cell response. This method includes administering to a subject in need thereof a therapeutically effective amount of at least one perturbagen selected from Table 3, or a variant thereof, wherein the at least one perturbagen is capable of changing a gene signature in a T cell. In some embodiments, the disease or disorder is characterized by immune tolerance. In some embodiments, the method further includes administering IL-2.

Administering related to this aspect of the disclosure can be done orally or parenterally. In some embodiments, the administering is done via intravenous, intramuscular, intraperitoneal, subcutaneous, intra-articular injection, or infusion route. In other embodiments, the administering is done via intraosseous injection or intraosseous infusion.

In some embodiments, the disease or disorder characterized by insufficient T cell response is a cancer. The cancer, e.g., can be a solid tumor or a liquid tumor. In some embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments, the cancer is a melanoma, optionally selected from metastatic melanoma, cutaneous melanoma, BRAF V600, Merkel cell carcinoma, cutaneous squamous cell carcinoma, a lung cancer, optionally selected from NSCLC, metastatic nonsquamous non-small cell lung cancer, metastatic small cell carcinoma, a head and neck cancer, optionally selected from head and neck squamous cell cancer, a bladder cancer, optionally selected from urothelial carcinoma, locally advanced or metastatic urothelial carcinoma, a breast cancer, optionally selected from triple-negative breast cancer), microsatellite Instability-high cancer, gastric cancer, optionally selected from gastric or gastroesophageal junction adenocarcinoma, metastatic colorectal cancer, cervical cancer, optionally selected from recurrent or metastatic cervical cancer, liver cancer, optionally selected from HCC, hematological disorder, optionally selected from primary mediastinal large B-cell lymphoma, and Hodgkin lymphoma.

In some embodiments, the cancer is characterized by a tumor expressing one or more of PD-1, PD-L1, PD-L2, CTLA-4, Tim-3, or LAG-3. In some embodiments, the cancer is poorly responsive or non-responsive to checkpoint inhibitor therapy or has presented as poorly responsive or non-responsive to checkpoint inhibitor therapy. The checkpoint inhibitor therapy is selected from, e.g., an antibody or antibody format specific for one of PD-1, PD-L1, PD-L2, CTLA-4, Tim-3, and LAG-3.

In some embodiments, the antibody or antibody format specific for PD-1 is selected from nivolumab, pembrolizumab, and pidilizumab. In embodiments, the antibody or antibody format specific for PD-L1 is selected from atezolizumab, avelumab, durvalumab, and BMS-936559. In other embodiments, the antibody or antibody format specific for CTLA-4 is selected from ipilimumab (YERVOY), tremelimumab, AGEN1884, and RG2077.

In some embodiments, the tumor is a less-immunogenic tumor and the methods of treatment described herein elicit a potent immune response in the less-immunogenic tumors.

In some embodiments, the tumor has reduced inflammation (“cold tumor”) and the methods of treatment described herein convert the tumor to a responsive, inflamed tumor (“hot tumor”). In embodiments, the cold tumor is characterized by one or more of an absence of T cells, lack of tumor antigens, APC deficit, absence of T cell priming/activation, and impaired trafficking of T cells to the tumor mass. In embodiments, the hot tumor is characterized by one or more of a presence of T cells, presence of tumor antigens, presence of APCs, presence of T cell priming/activation, and effective trafficking of T cells to the tumor mass.

In some embodiments, the method of treatment described herein makes the cancer responsive or more responsive to a checkpoint inhibitor therapy and, optionally one or more chemotherapeutic agents and/or radiotherapy.

In some embodiments, the subject of the methods of treatment described herein is predicted to be poorly responsive or non-responsive to the checkpoint inhibitor therapy based on expression of one or more of PD-1, PD-L1, or PD-L2, in a subject's biological specimen. In other embodiments, the subject is predicted to be poorly responsive or non-responsive to an agent that modulates one or more of PD-1, PD-L1, and PD-L2 based on low on expression of PD-1, PD-L1, and PD-L2 in a tumor specimen. In embodiments, the subject is predicted to be poorly responsive or non-responsive to an agent that modulates one or more of PD-1, PD-L1, and PD-L2 tumor proportion score (TPS) of less than about 49% for PD-L1 staining. The tumor proportion score (TPS) is a PD-L1 measurement which is applied, for example, to lung cancer, head and neck cancer and melanomas. PD-L1 protein expression is determined by using Tumor Proportion Score (TPS), which is the percentage of viable tumor cells showing partial or complete membrane staining.

In some aspects, the present disclosure is related to a method for treating or preventing cancer, comprising administering to a subject in need thereof a therapeutically effective amount of at least one perturbagen selected from Table 3, or a variant thereof, wherein the at least one perturbagen is capable of changing a gene signature in a T cell. In this method, the administering is done orally or parenterally. In some embodiments, the administration is done via intravenous, intramuscular, intraperitoneal, subcutaneous, intra-articular injection, and infusion route. In other embodiments, the administering is done via intraosseous injection or intraosseous infusion.

In some embodiments related to this aspect of the disclosure, the cancer is a solid tumor. In other embodiments, related to this aspect of the disclosure, the cancer is a liquid tumor. In some embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments, the cancer is a melanoma, optionally selected from metastatic melanoma, cutaneous melanoma, BRAF V600, Merkel cell carcinoma, cutaneous squamous cell carcinoma, a lung cancer, optionally selected from NSCLC, metastatic nonsquamous non-small cell lung cancer, metastatic small cell carcinoma, a head and neck cancer, optionally selected from head and neck squamous cell cancer, a bladder cancer, optionally selected from urothelial carcinoma, locally advanced or metastatic urothelial carcinoma, a breast cancer, optionally selected from triple-negative breast cancer), microsatellite Instability-high cancer, gastric cancer, optionally selected from gastric or gastroesophageal junction adenocarcinoma, metastatic colorectal cancer, cervical cancer, optionally selected from recurrent or metastatic cervical cancer, liver cancer, optionally selected from HCC, hematological disorder, optionally selected from primary mediastinal large B-cell lymphoma, and Hodgkin lymphoma. In some embodiments, the cancer is characterized by a tumor expressing one or more of PD-1, PD-L1, PD-L2, CTLA-4, Tim-3, or LAG-3. In some embodiments, the cancer is poorly responsive or non-responsive to checkpoint inhibitor therapy or has presented as poorly responsive or non-responsive to checkpoint inhibitor therapy. The checkpoint inhibitor therapy is selected from, e.g., an antibody or antibody format specific for one of PD-1, PD-L1, PD-L2, CTLA-4, Tim-3, and LAG-3.

In some embodiments, the antibody or antibody format specific for PD-1 is selected from nivolumab, pembrolizumab, and pidilizumab. In embodiments, the antibody or antibody format specific for PD-L1 is selected from atezolizumab, avelumab, durvalumab, and BMS-936559. In other embodiments, the antibody or antibody format specific for CTLA-4 is selected from ipilimumab (YERVOY), tremelimumab, AGEN1884, and RG2077.

In some embodiments, the tumor is a less-immunogenic tumor and the methods of treatment described herein elicit a potent immune response in the less-immunogenic tumors.

In some embodiments, the tumor has reduced inflammation (“cold tumor”) and the methods of treatment described herein convert the tumor to a responsive, inflamed tumor (“hot tumor”). In embodiments, the cold tumor is characterized by one or more of an absence of T cells, lack of tumor antigens, APC deficit, absence of T cell priming/activation, and impaired trafficking of T cells to the tumor mass. In embodiments, the hot tumor is characterized by one or more of a presence of T cells, presence of tumor antigens, presence of APCs, presence of T cell priming/activation and effective trafficking of T cells to the tumor mass.

In some embodiments, the method of treatment described herein makes the cancer responsive or more responsive to a checkpoint inhibitor therapy and, optionally one or more chemotherapeutic agents and/or radiotherapy.

In some embodiments, the subject of the methods of treatment described herein is predicted to be poorly responsive or non-responsive to the checkpoint inhibitor therapy based on expression of one or more of PD-1, PD-L1, or PD-L2, in a subject's biological specimen. In other embodiments, the subject is predicted to be poorly responsive or non-responsive to an agent that modulates one or more of PD-1, PD-L1, and PD-L2 based on low on expression of PD-1, PD-L1, and PD-L2 in a tumor specimen. In embodiments, the subject is predicted to be poorly responsive or non-responsive to an agent that modulates one or more of PD-1, PD-L1, and PD-L2 tumor proportion score (TPS) of less than about 49% for PD-L1 staining. The tumor proportion score (TPS) is a PD-L1 measurement which is applied, for example, to lung cancer, head and neck cancer and melanomas. PD-L1 protein expression is determined by using Tumor Proportion Score (TPS), which is the percentage of viable tumor cells showing partial or complete membrane staining. In some embodiments, the method improves clinical outcome or response to therapy with an anti-cancer agent as compared to clinical outcome or response to therapy in the absence of the perturbagen.

In one aspect, the present disclosure is related to a method for treating or preventing an infection, comprising administering to a subject in need thereof a therapeutically effective amount of at least one perturbagen selected from Table 3, or a variant thereof, wherein the at least one perturbagen is capable of changing a gene signature in a T cell. In some embodiments, the infection is selected from bacterial infections, viral infections, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections. In some embodiments, the method improves clinical outcome or response to therapy with an anti-infective agent or vaccine as compared to clinical outcome or response to therapy in the absence of the perturbagen. In some embodiments, the administering is oral or parenteral and is optionally selected from intravenous, intramuscular, intraperitoneal, subcutaneous, intra-articular injection and infusion.

In some aspects, the present disclosure is related to a method for making a therapeutic agent for a cancer or infection. This method includes the steps of (a) identifying a therapeutic agent for therapy using methods described herein and (b) formulating the therapeutic agent for the treatment of the disease or disorder.

In some embodiments, the method includes making a therapeutic agent for cancer. In embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma;

intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments, the method includes making a therapeutic agent for an infection, an infectious disease or disorder. In embodiments, the infection is selected from bacterial infections, viral infections, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

In embodiments, the administering is directed to the bone marrow of the patient. In embodiments, the administering is via intraosseous injection or intraosseous infusion. In embodiments, the administering the cell is via intravenous injection or intravenous infusion. In other embodiments, the administering of the cell is via intravenous injection or intravenous infusion. In some embodiments, the administering is simultaneously or sequentially to one or more mobilization agents.

In some embodiments, the methods described herein are useful for treatment of a disease or disorder characterized by an abnormal number of exhausted T cells.

In some embodiments, the methods described herein are where at least one perturbagen is administered on the basis of previously determining the patient exhibits an abnormal number of exhausted T cells or a disease or disorder characterized thereby.

In embodiments related to this aspect of the disclosure, the administering is directed to the bone marrow of the patient. In other embodiments, the administering is via intraosseous injection or intraosseous infusion. In embodiments, the administering the cell is via intravenous injection or intravenous infusion. In embodiments, the administering is simultaneously or sequentially to one or more mobilization agents. In embodiments, the administering occurs about once per day for one or more days. In embodiments, the administering occurs more than once per day for one or more days. In embodiments, the administering occurs at most once per day for one or more days. In embodiments, the administering occurs substantially continuously per administration period.

### Administration, Dosing, and Treatment Regimens

As examples, administration results in the delivery of one or more perturbagens disclosed herein into the bloodstream (via enteral or parenteral administration), or alternatively, the one or more perturbagens is administered directly to the site of T cell development, proliferation and/or maturation, i.e., in the bone marrow or in thymus. In some embodiments, the administering is done orally or parenterally. In embodiments, the administering is done via intravenous, intramuscular, intraperitoneal, subcutaneous, intra-articular injection, or infusion route.

Delivery of one or more perturbagens disclosed herein to the bone marrow may be via intravenous injection or intravenous infusion or via intraosseous injection or intraosseous infusion. Devices and apparatuses for performing these delivery methods are well known in the art.

Delivery of one or more perturbagens disclosed herein into the bloodstream via intravenous injection or intravenous infusion may follow or be contemporaneous with stem cell mobilization. In stem cell mobilization, certain drugs are used to cause the movement of stem cells from the bone marrow into the bloodstream. Once in the bloodstream, the stem cells are contacted with the one or more perturbagens and are able to alter a gene signature in a T cell, for example. Drugs and methods relevant to stem cell mobilization are well known in the art; see, e.g., Mohammadi et al, “Optimizing Stem Cells Mobilization Strategies to Ameliorate Patient Outcomes: A Review of Guide-lines and Recommendations.” Int. J. Hematol. Oncol. Stem Cell Res. 2017 Jan. 1; 11 (1): 78-88; Hopman and DiPersio “Advances in Stem Cell Mobilization.” Blood Review, 2014, 28 (1): 31-40; and Kim “Hematopoietic stem cell mobilization: current status and future perspective.” Blood Res. 2017 June; 52 (2): 79-81. The content of each of which is incorporated herein by reference in its entirety.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any perturbagen disclosed herein as well as the dosing schedule can depend on various parameters and factors, including, but not limited to, the specific perturbagen, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A perturbagen disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, e.g., the bone marrow, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

The dosage regimen utilizing any perturbagen disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed. Any perturbagen disclosed herein can be administered in a single daily dose, or the total daily dosage can be administered in divided doses of two, three or four times daily. Furthermore, any perturbagen disclosed herein can be administered continuously rather than intermittently throughout the dosage regimen.

### Pharmaceutical Compositions and Formulations

Aspects of the present disclosure include a pharmaceutical composition comprising a therapeutically effective amount of one or more perturbagens, as disclosed herein.

The perturbagens disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. In embodiments, the compositions disclosed herein are in the form of a pharmaceutically acceptable salt.

Further, any perturbagen disclosed herein can be administered to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any perturbagen disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference. In embodiments, the compositions, e.g., pharmaceutical compositions, disclosed herein are suspended in a saline buffer (including, without limitation TBS, PBS, and the like).

The present disclosure includes the disclosed perturbagens in various formulations of pharmaceutical compositions. Any perturbagens disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Where necessary, the pharmaceutical compositions comprising the perturbagens can also include a solubilizing agent. Also, the agents can be delivered with a suitable vehicle or delivery device as known in the art.

Combination therapies, comprising more than one perturbagen, can be co-delivered in a single delivery vehicle or delivery device.

Compositions for administration can optionally include a local anesthetic such as, for example, lignocaine to lessen pain at the site of the injection.

The pharmaceutical compositions comprising the perturbagens of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any perturbagens disclosed herein is formulated in accordance with routine procedures as a pharmaceutical composition adapted for a mode of administration disclosed herein.

### Other Aspects of the Present Disclosure

Embodiments associated with any of the above-disclosed aspects are likewise relevant to the below-mentioned aspects. In other words, each of the embodiments mentioned above for the above aspects may be revised/adapted to be applicable to the below aspects.

In some embodiments, the methods of treatments described herein include a step of selecting a subject. In such embodiments, the subject is selected by steps including: obtaining from the subject a sample of cells comprising a T cell; and contacting the sample of cells with least one perturbagen selected from Table 3, or a variant thereof, wherein the at least one perturbagen alters a gene signature in the sample of cells.

In some embodiments, the methods of treatments described herein include a step of selecting a subject. In such embodiments, the subject is selected by steps including: obtaining from the subject a sample of cells comprising a T cell; and contacting the sample of cells with at least one perturbagen capable of altering a gene signature in a T cell, wherein the at least one perturbagen increases in the sample of cells the expression and/or activity of one or more genes selected from Table 1 designated as an “up” gene in the gene directionality column of Table 1 and/or decreases in the sample of cells the expression and/or activity of one or more genes selected from Table 1 designated as an “up” gene in the gene directionality column of Table 1.

In embodiments, the at least one perturbagen increases in the sample of cells the expression and/or activity of one or more genes selected from Table 1 designated as an “up” gene in the gene directionality column of Table 1. In embodiments, the at least one perturbagen decreases in the sample of cells the expression and/or activity of one or more genes selected from Table 1 designated as a “down” gene in the gene directionality column of Table 1.

Yet another aspect of the present disclosure is a use of the perturbagen of Table 3, or a variant thereof in the manufacture of a medicament for treating a disease or disorder characterized by production of exhausted T cells, an increased rate of production of exhausted T cells, an increased rate of production of exhausted T cells, an abnormal number of exhausted T cells, or a high number of exhausted T cells.

In some aspects, the present disclosure is related to a method of identifying a candidate perturbation for reducing or reversing the conversion of a T cell into an exhausted T cell. This method includes i) exposing a starting population of T cells to a perturbation; ii) identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of T cells into exhausted T cells following exposure of the population of cells to the perturbation; and iii) identifying the perturbation as a candidate perturbation for reducing or reversing the conversion of a T cell thereof based on the perturbation signature, wherein the perturbation signature is an increase in expression and/or activity in the T cell of one or more genes selected from Table 1 designated as an “up” gene in the gene directionality column of Table 1, and/or a decrease in expression and/or activity in the T cell of one or more genes selected from Table 1 designated as a “down” gene in the gene directionality column of Table 1.

In some embodiments, the perturbation signature is an increase in expression and/or activity in the T cell of one or more genes selected from Table 1 designated as an “up” gene in the gene directionality column of Table 1.

In embodiments, the perturbation signature is a decrease in expression and/or activity in the T cell of one or more genes selected from Table 1 designated as a “down” gene in the gene directionality column of Table 1.

Yet another aspect of the present disclosure is a perturbagen capable of causing a change in a gene signature within naïve T cells, effector T cells or exhausted T cells. In an aspect, the present disclosure provides a perturbagen capable of causing a change in a cell fate of T cells. In another aspect, the present disclosure provides a perturbagen capable of causing a change in a gene signature and a change in a cell fate of T cells.

In yet another aspect, the present disclosure provides a pharmaceutical composition comprising any herein disclosed perturbagen. In a further aspect, the present disclosure provides a unit dosage form comprising an effective amount of the pharmaceutical composition comprising any herein disclosed perturbagen.

The instant disclosure also provides certain embodiments as follows:

Embodiment 1: A method for directing a change in cell state of a T cell in a subject, comprising: administering at least one perturbagen selected from Table 3, or a variant thereof to the subject, the perturbagen being capable of altering a gene signature in the T cell.

Embodiment 2: A method for directing a change in cell state of a T cell in a subject, comprising: administering at least one perturbagen capable of altering a gene signature in the T cell to the subject, wherein altering the gene signature comprises an increase in expression and/or activity in the T cell of one or more genes selected from Table 1 designated as an “up” gene in the gene directionality column of Table 1 and/or a decrease in expression and/or activity in the T cell of one or more genes selected from Table 1 designated as a “down” gene in the gene directionality column of Table 1.

Embodiment 3: The method of Embodiment 2, wherein altering the gene signature comprises an activation of a network module designated in the network module column of Table 1.

Embodiment 4: The method of Embodiment 3, wherein the activation of the network module comprises modulating expression and/or activity of 2 or more genes within a network module.

Embodiment 5: The method of Embodiment 4, wherein the activation of the network module comprises modulating expression and/or activity of all genes within a network module.

Embodiment 6: The method of Embodiment 2, wherein the activation of the network module comprises modulating expression and/or activity of 2 or more genes within 2 or more network modules.

Embodiment 7: A method for directing a change in cell state of a T cell in a subject, comprising: administering at least one perturbagen selected from Table 3, or a variant thereof to the subject, the perturbagen being capable of altering a gene signature in the T cell, wherein altering the gene signature comprises an increase in expression and/or activity in the T cell of one or more genes selected from Table 1 designated as an “up” gene in the gene directionality column of Table 1 and/or a decrease in expression and/or activity in the T cell of one or more genes selected from Table 1 designated as a “down” gene in the gene directionality column of Table 1.

Embodiment 8: The method of Embodiment 7, wherein altering the gene signature comprises an activation of a network module designated in the network module column of Table 1.

Embodiment 9: The method of Embodiment 8, wherein the activation of the network module comprises modulating expression and/or activity of 2 or more genes within a network module.

Embodiment 10: The method of Embodiment 9, wherein the activation of the network module comprises modulating expression and/or activity of all genes within a network module.

Embodiment 11: The method of Embodiment 10, wherein the activation of the network module comprises modulating expression and/or activity of 2 or more genes within 2 or more network modules.

Embodiment 12: The method of any one of Embodiments 1-11, wherein the T cell is selected from an effector T cell, an exhausted T cell, and a naïve T cell.

Embodiment 13: The method of any one of Embodiments 1-12, wherein the change in cell state is a prevention or reduction of a cellular transition to an exhausted T cell state.

Embodiment 14: The method of any one of Embodiments 1-12, wherein the change in cell state is a prevention or reduction of a transition of an effector T cell to an exhausted T cell state.

Embodiment 15: The method of any one of Embodiments 1-12, wherein the change in cell state is a prevention or reduction of a transition of a naïve T cell to an exhausted T cell state.

Embodiment 16: The method of any one of Embodiments 1-12, wherein the change in cell state is a stimulation or increase of a transition of an exhausted T cell to an effector T cell.

Embodiment 17: The method of any one of Embodiments 1-12, wherein the change in cell state is a stimulation or increase of a transition of a naïve T cell to an effector T cell.

Embodiment 18: The method of any one of Embodiments 1-12, wherein the change in cell state is a stimulation or increase of a cell death of an exhausted T cell.

Embodiment 19: The method of any one of Embodiments 1-12, wherein the change in cell state is a prevention or reduction of a cell death of an effector T cell and/or a naïve T cell.

Embodiment 20: The method of any one of Embodiments 1-12, wherein the change in cell state provides an increase in the number of one or more of effector T cells and naïve T cells.

Embodiment 21: The method of any one of Embodiments 1-12, wherein the change in cell state provides an increase in the number of one or more of effector T cells and naïve T cells relative to the number of effector T cells or naïve T cells obtained from a population of T cells that is not contacted with the at least one perturbagen.

Embodiment 22: The method of any one of Embodiments 1-12, wherein the change in cell state provides an increase in the number of one or more of effector T cells and naïve T cells relative to the number of effector T cells or naïve T cells obtained from a population of T cells prior to contacting with the at least one perturbagen.

Embodiment 23: The method of any one of Embodiments 1-12, wherein the change in cell state provides a decrease in the number of one or more of exhausted T cells.

Embodiment 24: The method of any one of Embodiments 1-12, wherein the change in cell state provides a decrease in the number of one or more of exhausted T cells relative to the number of exhausted T cells obtained from a population of T cells that is not contacted with the at least one perturbagen.

Embodiment 25: The method of any one of Embodiments 1-12, wherein the change in cell state provides a decrease in the number of one or more of exhausted T cells relative to the number of exhausted T cells obtained from a population of T cells prior to contacting with the at least one perturbagen.

Embodiment 26: The method of any one of Embodiments 1-12, wherein the ratio of the number of effector T cells and/or naïve T cells to the number of exhausted T cells is increased relative to the ratio obtained from a population of T cells that is not contacted with the at least one perturbagen.

Embodiment 27: The method of any one of Embodiments 1-12, wherein the ratio of the number of effector T cells and/or naïve T cells to the number of exhausted T cells is increased relative to the ratio obtained from a population of T cells prior to contacting with the at least one perturbagen.

Embodiment 28: The method of any one of Embodiments 20-22, wherein the increase in the number of effector T cells and/or naïve T cells is due in part to increased cell proliferation of the effector T cells and/or naïve T cells. Embodiment 29: The method of any one of Embodiments 20-22, wherein the increase in the number of effector T cells and/or naïve T cells is due in part to an increased lifespan of the effector T cells and/or naïve T cells.

Embodiment 30: The method of any one of Embodiments 20-22, wherein the increase in the number of effector T cells and/or naïve T cells is due in part to reduced cell death among the effector T cells and/or naïve T cells.

Embodiment 31: The method of any one of Embodiments 23-25, wherein the decrease in the number of exhausted T cells is due in part to decreased cell proliferation of the T cells.

Embodiment 32: The method of any one of Embodiments 23-25, wherein the decrease in the number of exhausted T cells is due in part to a decreased lifespan of the T cells.

Embodiment 33: The method of any one of Embodiments 23-25, wherein the decrease in the number of exhausted T cells is due in part to increased cell death among the T cells.

Embodiment 34: The method of any one of Embodiments 20-22, wherein the number of effector T cells and/or naïve T cells is increased after contacting the population of T cells with the at least one perturbagen.

Embodiment 35: The method of any one of Embodiments 23-25, wherein the number of exhausted T cells is decreased after contacting the population of T cells with the at least one perturbagen.

Embodiment 36: The method of any one of Embodiments 1-35, wherein the method promotes the formation of a T cells that are responsive to antigen, the antigen optionally being presented by a professional antigen-presenting cell.

Embodiment 37: The method of any one of Embodiments 1-36, wherein the method promotes the formation of T cells that are responsive to both TCR engagement and co-stimulation.

Embodiment 38: The method of any one of Embodiments 1-37, wherein the method promotes the formation of T cells that are capable of elaborating cytokines.

Embodiment 39: The method of Embodiment 38, wherein the cytokines are selected from IL-2, IFNγ, TNFα, and a CC chemokine (or β-chemokine).

Embodiment 40: The method of any one of Embodiments 1-39, wherein the method promotes the formation of T cells that are capable of proliferating, e.g. in the presence of TCR engagement and co-stimulation.

Embodiment 41: The method of any one of Embodiments 1-40, wherein the method promotes the formation of T cells that are anabolic.

Embodiment 42: The method of any one of Embodiments 1-40, wherein the method promotes the formation of T cells that are cytotoxic.

Embodiment 43: The method of any one of Embodiments 1-40, wherein the method promotes the formation of effector T cells.

Embodiment 44: The method of any one of Embodiments 1-40, wherein the method represses the formation of a T cells that are not responsive or poorly responsive to antigen.

Embodiment 45: The method of Embodiment 44, wherein the antigen is being presented by a professional antigen-presenting cell.

Embodiment 46: The method of any one of Embodiments 1-39, wherein the method represses the formation of T cells that are not responsive or poorly responsive to both TCR engagement and co-stimulation.

Embodiment 47: The method of any one of Embodiments 1-39, wherein the method represses the formation of T cells that of a T cells that are incapable or poorly capable of elaborating cytokines.

Embodiment 48: The method of Embodiment 47, wherein the cytokines are selected from IL-2, IFNγ, TNFα, and a CC chemokine (or β-chemokine).

Embodiment 49: The method of any one of Embodiments 1-39, wherein the method represses the formation of T cells that are incapable or poorly capable of proliferating, e.g. in the presence of signal 1 and signal 2.

Embodiment 50: The method of Embodiment 49, wherein the method represses the formation of T cells that are not anabolic or minimally anabolic.

Embodiment 51: The method of any one of Embodiments 1-39, wherein the method represses the formation of T cells that are not cytotoxic or minimally cytotoxic.

Embodiment 52: The method of any one of Embodiments 1-51, wherein the method represses the formation of exhausted T cells.

Embodiment 53: The method of any one of Embodiments 1-52, wherein the method reduces the number of T cells which demonstrate expression or increased expression of one of more of PD-1, PD-L1, PD-L2, CTLA-4, LAG-3, TIM-3, 2B4/CD244/SLAMF4, CD160, and TIGIT.

Embodiment 54: The method of any one of Embodiments 1-52, wherein the method prevents or reduces formation of T cells which demonstrate expression or increased expression of one of more of PD-1, PD-L1, PD-L2, CTLA-4, LAG-3, TIM-3, 2B4/CD244/SLAMF4, CD160, and TIGIT.

Embodiment 55: The method of Embodiment 52, wherein the exhausted T cells demonstrate expression or increased expression of one of more of PD-1, PD-L1, PD-L2, CTLA-4, LAG-3, TIM-3, 2B4/CD244/SLAMF4, CD160, and TIGIT.

Embodiment 56: The method of Embodiment 52, wherein the exhausted T cells demonstrate expression or increased expression of a transcription factor selected from TOX, TCF1, NR4A and NFAT.

Embodiment 57: The method of Embodiment 52, wherein the exhausted T cells demonstrate a loss or reduction of IL-2 productive capacity, proliferative capacity, and/or cytolytic activity.

Embodiment 58: The method of Embodiment 52, wherein the exhausted T cells demonstrate a loss or reduction of TNFα, IFNγ, and CC chemokine (or β-chemokine) signaling.

Embodiment 59: The method of Embodiment 52, wherein the exhausted T cells demonstrate degranulation and/or increased or high expression of granzyme B.

Embodiment 60: The method of Embodiment 52, wherein the exhausted T cells demonstrate poor responsiveness to IL-7 and/or IL-15.

Embodiment 61: The method of any one of Embodiments 1-60, wherein the method further comprises administering IL-2.

Embodiment 62: The method of any of Embodiments 1-61, wherein the T cells are CD8+ T cells.

Embodiment 63: The method of any of Embodiments 1-62, wherein the T cells are CD4+ T cells.

Embodiment 64: The method of any of Embodiments 1 or 7-63, wherein the at least one perturbagen selected from Table 3, or a variant thereof, comprises at least 2, at least 3, at least 4, or at least 5, at least 6, at least 7, at least 8, at least 9, at least 10, at least 11, at least 12, at least 13, at least 14, at least 15, at least 16, at least 17, at least 18, at least 19, and at least 20 perturbagens selected from Table 3, or variants thereof.

Embodiment 65: The method of any of Embodiments 2-64, wherein the one or more genes selected from Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, and 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more genes are selected from Table 1 designated as an “up” gene in the gene directionality column of Table 1.

Embodiment 66: The method of Embodiment 65, wherein the one or more genes selected from Table 1 comprises at least one of NFKBIA, TSC22D3, ZFP36, ARHGEF2, FHL2, STMN1, CDC25B, CCND3, TMEM109, E2F2, SCP2, PDLIM1, CORO1A, ATP11B, SATB1, CXCR4, ARL4C, CTSD, CD44, ZMIZ1, TBXA2R, GNA15, PRKCQ, RHOA, SLC25A4, PRUNE, CDC42, TIMP2, FAM69A, NRAS, BHLHE40, DNAJC15, GNAI2, DHRS7, CYTH1, ADGRE5, IGF2R, ADRB2, EIF4EBP1, FAS, MRPS16, TMEM50A, S100A4, RSU1, SPTAN1, S100A13, RAC2, REEP5, MACF1, PLP2, and TWF2.

Embodiment 67: The method of Embodiments 2-66, wherein the one or more genes selected from Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, and 20 or more, 21 or more, 22 or more, 23 or more, 24 or more, 25 or more, 26 or more, 27 or more, 28 or more, 29 or more, 30 or more, or 31 or more genes selected from Table 1 designated as a “down” gene in the gene directionality column of Table 1.

Embodiment 68: The method of Embodiment 67, wherein the one or more genes selected from Table 1 comprises at least one of STAT1, DUSP6, INPP1, PSMB8, MLEC, ID2, RGS2, UBE2L6, SSBP2, PRKCH, ALDOA, ADGRG1, MFSD10, HERC6, CEP57, FBXL12, ICAM1, GLRX, PSME2, MYCBP2, IKZF1, PSMB10, PSME1, EVL, MBNL 1, FYN, DNAJB6, FOXO3, TSPAN3, SYNE2, and RPS6.

Embodiment 69: The method of any of Embodiments 1-68, wherein the subject is a human.

Embodiment 70: The method of Embodiment 69, wherein the human is an adult human.

Embodiment 71: The method of any of Embodiments 1-70, wherein the administering is oral or parenteral.

Embodiment 72: The method of Embodiment 71, wherein the administering is done via intravenous, intramuscular, intraperitoneal, subcutaneous, intra-articular injection, or infusion route.

Embodiment 73: The method of Embodiment 71, wherein the administering is via intraosseous injection or intraosseous infusion.

Embodiment 74: The method of any of Embodiments 1-73, wherein the change in cell state is within the tumor microenvironment, if the subject is afflicted with cancer.

Embodiment 75: The method of Embodiment 74, wherein the change in cell state is within the thymus.

Embodiment 76: The method of Embodiment 74, wherein the change in cell state is within the bone marrow.

Embodiment 77: A perturbagen for use in the method of any of Embodiments 1-76.

Embodiment 78: A pharmaceutical composition comprising the perturbagen of Embodiment 77.

Embodiment 79: A method for treating a disease or disorder characterized by insufficient T cell response, comprising administering to a subject in need thereof a therapeutically effective amount of at least one perturbagen selected from Table 3, or a variant thereof, wherein the at least one perturbagen is capable of changing a gene signature in a T cell.

Embodiment 80: The method of Embodiment 79, wherein the disease or disorder is characterized by immune tolerance.

Embodiment 81: The method of any one of Embodiments 79 or 80, wherein the administering is oral or parenteral.

Embodiment 82: The method of Embodiment 81, wherein the administering is done via an intravenous, intramuscular, intraperitoneal, subcutaneous, intra-articular injection, or infusion route.

Embodiment 83: The method of Embodiment 81, wherein the administering is done via intraosseous injection or intraosseous infusion.

Embodiment 84: The method of any one of Embodiments 79-83, wherein the method further comprises administering IL-2.

Embodiment 85: The method of any one of Embodiments 79-84, wherein the disease or disorder characterized by insufficient T cell response is a cancer.

Embodiment 86: The method of Embodiment 85, wherein the cancer is a solid tumor.

Embodiment 87: The method of Embodiment 85, wherein the cancer is a liquid tumor.

Embodiment 88: The method of one of Embodiments 85-87, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

Embodiment 89: The method of one of Embodiments 85-88, wherein the cancer is a melanoma, optionally selected from metastatic melanoma, cutaneous melanoma, BRAF V600, Merkel cell carcinoma, cutaneous squamous cell carcinoma, a lung cancer, optionally selected from NSCLC, metastatic nonsquamous non-small cell lung cancer, metastatic small cell carcinoma, a head and neck cancer, optionally selected from head and neck squamous cell cancer, a bladder cancer, optionally selected from urothelial carcinoma, locally advanced or metastatic urothelial carcinoma, a breast cancer, optionally selected from triple-negative breast cancer), microsatellite Instability-high cancer, gastric cancer, optionally selected from gastric or gastroesophageal junction adenocarcinoma, metastatic colorectal cancer, cervical cancer, optionally selected from recurrent or metastatic cervical cancer, liver cancer, optionally selected from HCC, hematological disorder, optionally selected from primary mediastinal large B-cell lymphoma, and Hodgkin lymphoma.

Embodiment 90: The method of one of Embodiments 85-89, wherein the cancer is characterized by a tumor expressing one or more of PD-1, PD-L1, PD-L2, CTLA-4, Tim-3, or LAG-3.

Embodiment 91: The method of one of Embodiments 85-90, wherein the cancer is poorly responsive or non-responsive to checkpoint inhibitor therapy or has presented as poorly responsive or non-responsive to checkpoint inhibitor therapy.

Embodiment 92: The method of Embodiment 91, wherein the checkpoint inhibitor therapy is selected from an antibody or antibody format specific for one of PD-1, PD-L1, PD-L2, CTLA-4, Tim-3, and LAG-3.

Embodiment 93: The method of Embodiment 92, wherein the antibody or antibody format specific for PD-1 is selected from nivolumab, pembrolizumab, and pidilizumab.

Embodiment 94: The method of Embodiment 92, wherein the antibody or antibody format specific for PD-L1 is selected from atezolizumab, avelumab, durvalumab, and BMS-936559.

Embodiment 95: The method of Embodiment 92, wherein the antibody or antibody format specific for CTLA-4 is selected from ipilimumab (YERVOY), tremelimumab, AGEN1884, and RG2077.

Embodiment 96: The method of Embodiment 90, wherein the tumor is a less-immunogenic tumor and the method elicits a potent immune response in the less-immunogenic tumors.

Embodiment 97: The method of Embodiment 90, wherein the tumor has reduced inflammation (“cold tumor”) and the method converts the tumor to a responsive, inflamed tumor (“hot tumor”).

Embodiment 98: The method of Embodiment 91, wherein the method makes the cancer responsive or more responsive to a checkpoint inhibitor therapy and, optionally one or more chemotherapeutic agents and/or radiotherapy.

Embodiment 99: The method of Embodiment 91, wherein the subject is predicted to be poorly responsive or non-responsive to the checkpoint inhibitor therapy based on expression of one or more of PD-1, PD-L1, or PD-L2, in a subject's biological specimen.

Embodiment 100: The method of Embodiment 90, wherein the subject is predicted to be poorly responsive or non-responsive to an agent that modulates one or more of PD-1, PD-L1, and PD-L2 based on low on expression of PD-1, PD-L1, and PD-L2 in a tumor specimen.

Embodiment 101: The method of Embodiment 90, wherein the subject is predicted to be poorly responsive or non-responsive to an agent that modulates one or more of PD-1, PD-L1, and PD-L2 tumor proportion score (TPS) of less than about 49% for PD-L1 staining.

Embodiment 102: The method of any of Embodiments 79-101, wherein the subject is selected by steps comprising: obtaining from the subject a sample of cells comprising a T cell; and contacting the sample of cells with least one perturbagen selected from Table 3, or a variant thereof, wherein the at least one perturbagen alters a gene signature in the sample of cells.

Embodiment 103: The method of any of Embodiments 79-101, wherein the subject is selected by steps comprising: obtaining from the subject a sample of cells comprising a T cell; and contacting the sample of cells with at least one perturbagen capable of altering a gene signature in a T cell, wherein the at least one perturbagen increases in the sample of cells the expression and/or activity of one or more genes selected from Table 1 designated as an “up” gene in the gene directionality column of Table 1 and/or decreases in the sample of cells the expression and/or activity of one or more genes selected from Table 1 designated as a “down” gene in the gene directionality column of Table 1.

Embodiment 104: The method of Embodiment 103, wherein altering the gene signature comprises an activation of a network module designated in the network module column of Table 1.

Embodiment 105: The method of Embodiment 104, wherein the activation of the network module designated in the network module column of Table 1 comprises modulating expression and/or activity of 2 or more genes within a network module.

Embodiment 106: The method of Embodiment 105, wherein the activation of the network module designated in the network module column of Table 1 comprises modulating expression and/or activity of all of the genes within a network module.

Embodiment 107: The method of Embodiment 106, wherein the activation of the network module designated in the network module column of Table 1 comprises modulating expression and/or activity of 2 or more genes within 2 or more network modules.

Embodiment 108: A method for treating or preventing cancer, comprising administering to a subject in need thereof a therapeutically effective amount of at least one perturbagen selected from Table 3, or a variant thereof, wherein the at least one perturbagen is capable of changing a gene signature in a T cell.

Embodiment 109: The method of Embodiment 108, wherein the administering is oral or parenteral, optionally selected from intravenous, intramuscular, intraperitoneal, subcutaneous, intra-articular injection, and infusion.

Embodiment 110: The method of Embodiment 109, wherein the administering is via intraosseous injection or intraosseous infusion.

Embodiment 111: The method of Embodiment 108, wherein the cancer is a solid tumor.

Embodiment 112: The method of Embodiment 108, wherein the cancer is a liquid tumor.

Embodiment 113: The method of any of Embodiments 108-112, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

Embodiment 114: The method of any of Embodiments 108-113, wherein the cancer is a melanoma, optionally selected from metastatic melanoma, cutaneous melanoma, BRAF V600, Merkel cell carcinoma, cutaneous squamous cell carcinoma, a lung cancer, optionally selected from NSCLC, metastatic nonsquamous non-small cell lung cancer, metastatic small cell carcinoma, a head and neck cancer, optionally selected from head and neck squamous cell cancer, a bladder cancer, optionally selected from urothelial carcinoma, locally advanced or metastatic urothelial carcinoma, a breast cancer, optionally selected from triple-negative breast cancer), microsatellite Instability-high cancer, gastric cancer, optionally selected from gastric or gastroesophageal junction adenocarcinoma, metastatic colorectal cancer, cervical cancer, optionally selected from recurrent or metastatic cervical cancer, liver cancer, optionally selected from HCC, hematological disorder, optionally selected from primary mediastinal large B-cell lymphoma, and Hodgkin lymphoma.

Embodiment 115: The method of any of Embodiments 108-114, wherein the cancer is characterized by a tumor expressing one or more of PD-1, PD-L1, PD-L2, CTLA-4, Tim-3, or LAG-3.

Embodiment 116: The method of any of Embodiments 108-115, wherein the cancer is poorly responsive or non-responsive to checkpoint inhibitor therapy or has presented as poorly responsive or non-responsive to checkpoint inhibitor therapy.

Embodiment 117: The method of Embodiment 116, wherein the checkpoint inhibitor therapy is selected from an antibody or antibody format specific for one of PD-1, PD-L1, PD-L2, CTLA-4, Tim-3, or LAG-3.

Embodiment 118: The method of Embodiment 117, wherein the antibody or antibody format specific for PD-1 is selected from nivolumab, pembrolizumab, and pidilizumab.

Embodiment 119: The method of Embodiment 117, wherein the antibody or antibody format specific for PD-L1 is selected from atezolizumab, avelumab, durvalumab, and BMS-936559.

Embodiment 120: The method of Embodiment 117, wherein the antibody or antibody format specific for CTLA-4 is selected from ipilimumab (YERVOY), tremelimumab, AGEN1884, and RG2077.

Embodiment 121: The method of any of Embodiments 108-120, wherein the method elicits a potent immune response in less-immunogenic tumors.

Embodiment 122: The method of any of Embodiments 108-121, wherein the method converts a tumor with reduced inflammation (“cold tumor”) to a responsive, inflamed tumor (“hot tumor”).

Embodiment 123: The method of any of Embodiments 108-122, wherein the method makes the cancer responsive or more responsive to a checkpoint inhibitor therapy and, optionally one or more chemotherapeutic agents and/or radiotherapy.

Embodiment 124: The method of any of Embodiments 108-123, wherein the subject is predicted to be poorly responsive or non-responsive to the checkpoint inhibitor therapy based on expression of one or more of PD-1, PD-L1, or PD-L2, in a subject's biological specimen.

Embodiment 125: The method of Embodiment 124, wherein the subject is predicted to be poorly responsive or non-responsive to an agent that modulates one or more of PD-1, PD-L1, and PD-L2 based on low on expression of PD-1, PD-L1, and PD-L2 in a tumor specimen.

Embodiment 126: The method of Embodiment 124, wherein the subject is predicted to be poorly responsive or non-responsive to an agent that modulates one or more of PD-1, PD-L1, and PD-L2 tumor proportion score (TPS) of less than about 49% for PD-L1 staining.

Embodiment 127: The method of any of Embodiments 108-126, wherein the method improves clinical outcome or response to therapy with an anti-cancer agent as compared to clinical outcome or response to therapy in the absence of the perturbagen.

Embodiment 128: A method for treating or preventing an infection, comprising administering to a subject in need thereof a therapeutically effective amount of at least one perturbagen selected from Table 3, or a variant thereof, wherein the at least one perturbagen is capable of changing a gene signature in a T cell.

Embodiment 129: The method of Embodiment 128, wherein the infection is selected from bacterial infections, viral infections, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

Embodiment 130: The method of Embodiment 128, wherein the method improves clinical outcome or response to therapy with an anti-infective agent or vaccine as compared to clinical outcome or response to therapy in the absence of the perturbagen.

Embodiment 131: The method of Embodiment 128, wherein the administering is oral or parenteral, optionally selected from intravenous, intramuscular, intraperitoneal, subcutaneous, intra-articular injection and infusion.

Embodiment 132: A method of identifying a candidate perturbation for reducing or reversing the conversion of a T cell into an exhausted T cell, the method comprising: exposing the starting population of T cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of T cells into exhausted T cells following exposure of the population of cells to the perturbation; and identifying the perturbation as a candidate perturbation for reducing or reversing the conversion of a T cell thereof based on the perturbation signature; wherein the perturbation signature is an increase in expression and/or activity in the T cell of one or more genes selected from Table 1 designated as an “up” gene in the gene directionality column of Table 1, and/or a decrease in expression and/or activity in the T cell of one or more genes selected from Table 1 designated as a “down” gene in the gene directionality column of Table 1.

Embodiment 133: The method of Embodiment 132, wherein altering the perturbation signature comprises an activation of a network module designated in the network module column of Table 1.

Embodiment 134: The method of Embodiment 133, wherein the activation of the network module designated in the network module column of Table 1 comprises modulating expression and/or activity of 2 or more genes within a network module.

Embodiment 135: The method of Embodiment 134, wherein the activation of the network module designated in the network module column of Table 1 comprises modulating expression and/or activity of all of the genes within a network module.

Embodiment 136: The method of Embodiment 135, wherein the activation of the network module designated in the network module column of Table 1 comprises modulating expression and/or activity of 2 or more genes within 2 or more network modules.

Embodiment 137: A method for making a therapeutic agent for a cancer or infection, comprising: (a) identifying a candidate perturbation according to the method of Embodiment 132, and (b) formulating the candidate perturbation as a therapeutic agent for the treatment of the disease or disorder.

Embodiment 138: The method of Embodiment 137, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

Embodiment 139: The method of Embodiment 137, wherein the infection is selected from bacterial infections, viral infections, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

Embodiment 139.1: A method for directing a change in cell state of a T cell in a subject, comprising: administering at least one perturbagen capable of altering a gene signature in the T cell to the subject, wherein altering the gene signature comprises an increase in expression and/or activity in the T cell of one or more genes selected from Table 1 designated as an “up” gene in the gene directionality column of Table 1 and/or a decrease in expression and/or activity in the T cell of one or more genes selected from Table 1 designated as a “down” gene in the gene directionality column of Table 1; wherein the change in cell state is a prevention or reduction of a cellular transition to an exhausted T cell state; and wherein the T cell is selected from an effector T cell, an exhausted T cell, and a naïve T cell.

Embodiment 139.2: The method of Embodiment 139.1, wherein the change in cell state is a prevention or reduction of a transition of an effector T cell to an exhausted T cell state.

Embodiment 139.3: The method of Embodiment 139.1, wherein the at least one perturbagen is selected from Table 3, or a variant thereof.

Embodiment 139.4: The method of Embodiment 139.3, wherein the at least one perturbagen selected from Table 3 comprises at least 2, at least 3, at least 4, or at least 5, at least 6, at least 7, at least 8, at least 9, at least 10, at least 11, at least 12, at least 13, at least 14, at least 15, at least 16, at least 17, at least 18, at least 19, and at least 20 perturbagens selected from Table 3, or variants thereof.

Embodiment 139.5: The method of Embodiment 139.1, wherein the T cells are CD8+ T cells.

Embodiment 139.6: The method of Embodiment 139.1, wherein the one or more genes selected from Table 1 comprises at least one of NFKBIA, TSC22D3, ZFP36, ARHGEF2, FHL2, STMN1, CDC25B, CCND3, TMEM109, E2F2, SCP2, PDLIM1, CORO1A, ATP11B, SATB1, CXCR4, ARL4C, CTSD, CD44, ZMIZ1, TBXA2R, GNA15, PRKCQ, RHOA, SLC25A4, PRUNE, CDC42, TIMP2, FAM69A, NRAS, BHLHE40, DNAJC15, GNAI2, DHRS7, CYTH1, ADGRE5, IGF2R, ADRB2, EIF4EBP1, FAS, MRPS16, TMEM50A, S100A4, RSU1, SPTAN1, S100A13, RAC2, REEP5, MACF1, PLP2, and TWF2.

Embodiment 139.7: The method of Embodiment 139.1, wherein the one or more genes selected from Table 1 comprises at least one of STAT1, DUSP6, INPP1, PSMB8, MLEC, ID2, RGS2, UBE2L6, SSBP2, PRKCH, ALDOA, ADGRG1, MFSD10, HERC6, CEP57, FBXL12, ICAM1, GLRX, PSME2, MYCBP2, IKZF1, PSMB10, PSME1, EVL, MBNL1, FYN, DNAJB6, FOXO3, TSPAN3, SYNE2, and RPS6.

### Ex-Vivo Methods

### Genes Signatures

Cell state transitions (i.e., a transition in a cell's state from a first cell state to a second cell state) are characterized by a change in expression of genes in the cell. Changes in gene expression may be quantified as, e.g., an increase in mRNA expressed for a specific gene or a decrease in mRNA expressed for another specific gene; especially significant here may be mRNAs that encode transcription factors. Collectively, the sum of multiple differences in gene expression between one cell type or cells of one state relative to another cell type or cells of another state are referred to herein as a gene signature. For instance, a gene signature, in various embodiments, includes the sum of multiple differences in gene expression between non-exhausted and exhausted T cells.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may be useful to first characterize the cell type or cells of a specific state by surface proteins that are characteristic of the cell type or cells of a specific state.

Knowing the gene signature for each cell type or cells of a specific state provides insight into what genes impact or are associated with the process of transition to other cell types.

Genes that are differentially expressed and positively associated with the promotion of non-exhausted T cells ex vivo are listed in Table 2. The genes listed in Table 2 show an increase or decrease in expression and/or activity in the cell state change.

TABLE 2
Genes showing an increase in expression
in the cell state change ex vivo.

| | | | | Network |
|---|---|---|---|---|
| | Gene | Gene EntrezID | Gene Directionality | Module |
| 0 | MYC | 4609 | up | 0 |
| 1 | CDK6 | 1021 | down | 0 |
| 2 | MTHFD2 | 10797 | down | 0 |
| 3 | ID2 | 3398 | down | 0 |
| 4 | SCCPDH | 51097 | down | 0 |
| 5 | SLC25A46 | 91137 | down | 0 |
| 6 | ETFB | 2109 | down | 0 |
| 7 | TES | 26136 | up | 1 |
| 8 | CXCR4 | 7852 | up | 1 |
| 9 | HLA-DRA | 3122 | down | 1 |
| 10 | CHN1 | 1123 | down | 1 |
| 11 | RAB27A | 5873 | down | 1 |
| 12 | TBXA2R | 6915 | down | 1 |
| 13 | NFKB2 | 4791 | down | 1 |
| 14 | IGFBP3 | 3486 | up | 2 |
| 15 | PRSS23 | 11098 | up | 2 |
| 16 | SYPL1 | 6856 | up | 2 |
| 17 | ITGAE | 3682 | down | 2 |
| 18 | SMC4 | 10051 | down | 2 |
| 19 | STMN1 | 3925 | down | 2 |
| 20 | CYB561 | 1534 | up | 3 |
| 21 | CCNH | 902 | up | 3 |
| 22 | GATA3 | 2625 | down | 3 |
| 23 | ETS1 | 2113 | down | 3 |
| 24 | IQGAP1 | 8826 | down | 3 |
| 25 | CAT | 847 | down | 3 |
| 26 | RALA | 5898 | down | 4 |
| 27 | TSC22D3 | 1831 | down | 4 |
| 28 | CBLB | 868 | down | 4 |
| 29 | INPP4B | 8821 | down | 4 |
| 30 | PLSCR1 | 5359 | down | 4 |
| 31 | NUSAP1 | 51203 | down | 4 |
| 32 | XBP1 | 7494 | up | 5 |
| 33 | RGS2 | 5997 | down | 5 |
| 34 | EVL | 51466 | down | 5 |
| 35 | PSMB8 | 5696 | down | 5 |
| 36 | HERPUD1 | 9709 | down | 5 |
| 37 | RPS6 | 6194 | up | 6 |
| 38 | ADRB2 | 154 | up | 6 |
| 39 | GDPD5 | 81544 | up | 6 |
| 40 | APBB2 | 323 | down | 6 |
| 41 | MIF | 268 | down | 6 |
| 42 | SORBS3 | 10174 | up | 7 |
| 43 | SQSTM1 | 8878 | down | 7 |
| 44 | PGAM1 | 5223 | down | 7 |
| 45 | TWF2 | 11344 | down | 7 |
| 46 | DRAP1 | 10589 | down | 7 |
| 47 | ZFP36 | 55552 | up | 8 |
| 48 | FOS | 2353 | up | 8 |
| 49 | ETV1 | 2115 | down | 8 |
| 50 | CCNA1 | 8900 | down | 8 |
| 51 | HTRA1 | 5654 | down | 8 |
| 52 | DUSP4 | 1846 | down | 9 |
| 53 | GAPDH | 2597 | down | 9 |
| 54 | RPA3 | 6119 | down | 9 |
| 55 | ADGRG1 | 9289 | down | 9 |
| 56 | ACOT9 | 23597 | down | 9 |
| 57 | PXN | 5829 | up | 10 |
| 58 | SLC25A4 | 291 | up | 10 |
| 59 | DSG2 | 1829 | up | 10 |
| 60 | SATB1 | 6304 | up | 10 |
| 61 | CALM3 | 801 | down | 10 |
| 62 | IER3 | 8870 | up | 11 |
| 63 | SOX4 | 6659 | down | 11 |
| 64 | HMOX1 | 3162 | down | 11 |
| 65 | RHOA | 387 | down | 11 |
| 66 | SSBP2 | 23635 | up | 12 |
| 67 | S100A4 | 6275 | down | 12 |
| 68 | ANKRD10 | 55608 | down | 12 |
| 69 | FCHO1 | 23149 | down | 12 |
| 70 | RPS5 | 6193 | up | 13 |
| 71 | ATP1B1 | 481 | up | 13 |
| 72 | KDM5B | 10765 | down | 13 |
| 73 | SPTAN1 | 6709 | down | 13 |
| 74 | GADD45B | 4616 | up | 14 |
| 75 | CTSD | 1509 | down | 14 |
| 76 | HLA-DMA | 3108 | down | 14 |
| 77 | FGFR4 | 2264 | down | 14 |
| 78 | SLC1A4 | 6509 | down | 15 |
| 79 | HSPB1 | 3315 | down | 15 |
| 80 | CDKN2A | 1029 | down | 15 |
| 81 | STAT3 | 6774 | down | 16 |
| 82 | RAC2 | 5880 | down | 16 |
| 83 | TIAM1 | 7074 | down | 16 |
| 84 | RALGDS | 5900 | down | 17 |
| 85 | EZH2 | 2146 | down | 17 |

In Table 2 and associated embodiments:
“Gene ID”: at the time of filing the present disclosure, the World Wide Web at ncbi.nlm.nih.gov/gene provides a description of and the nucleic acid sequence for each GeneID listed in Table 2; the contents of each of which is incorporated herein by reference in its entirety.
“Up” indicates a gene for which an increase in expression and/or activity in the T cell is associated with the gene signature.
“Down” indicates a gene for which a decrease in expression and/or activity in the T cell is associated with the gene signature.
A “network module” (sometimes also referred to as “module”) is a set of genes whose activity and/or expression are mutually predictive and, individually and collectively, are correlated with regard to a cell state change, which correlation may be positive or negative. That is, a module may contain genes that are positively associated with the cell state transition-such that an increase in expression and/or activity of the gene associated with the cell state transition; as well as genes that are negatively associated with the cell state transition such that a decrease in expression and/or activity of the gene associated with the cell state transition.

In certain embodiments, a network module includes genes in addition (or substituted for) to those exemplified in Table 2, which should be viewed as illustrative and not limiting unless expressly provided, namely with genes with correlated expression. A correlation, e.g., by the method of Pearson or Spearman, is calculated between a query gene expression profile for the desired cell state transition and one or more of the exemplary genes recited in the module. Those genes with a correlation with one or more genes of the module of at significance level below p=0.05 (e.g., 0.04, 0.03, 0.02, 0.01, 0.005, 0.001, 0.0005, 0.0001, or less) can be added to, or substituted for, other genes in the module.

“Activation of a network module” refers to a perturbation that modulates expression and/or activity of 2 or more genes (e.g., 3, 4, 5, 6 . . . genes; or about 10, 20, 25, 30, 40, 50, 60, 70, 75, 80, 85, 90, 95, or 100%) within a module, which modulation may be an increase or decrease in expression and/or activity of the gene as consonant with the modules described in Table 2. In certain embodiments, a perturbation activates multiple network modules for the desired cell state transition, such as 2, 3, 4, 5, 6, 7, 8, 9, 10, 11, 12, or all 13 modules.

In some embodiments, one or more genes of network module 0 are modulated. In some embodiments, the present disclosure relates to the activation of network module 0, e.g., one or more of (inclusive of all of) MYC, CDK6, MTHFD2, ID2, SCCPDH, SLC25A46, and ETFB.

In some embodiments, one or more genes of network module 1 are modulated. In some embodiments, the present disclosure relates to the activation of network module 1, e.g., one or more of (inclusive of all of) TES, CXCR4, HLA-DRA, CHN1, RAB27A, TBXA2R, and NFKB2.

In some embodiments, one or more genes of network module 2 are modulated. In some embodiments, the present disclosure relates to the activation of network module 2, e.g., one or more of (inclusive of all of) IGFBP3, PRSS23, SYPL1, ITGAE, SMC4, and STMN1.

In some embodiments, one or more genes of network module 3 are modulated. In some embodiments, the presents relate to the activation of network module 3, e.g., one or more of (inclusive of all of) CYB561, CCNH, GATA3, ETS1, IQGAP1, and CAT.

In some embodiments, one or more genes of network module 4 are modulated. In some embodiments, the presents relate to the activation of network module 4, e.g., one or more of (inclusive of all of) RALA, TSC22D3, CBLB, INPP4B, PLSCR1, and NUSAP1.

In some embodiments, one or more genes of network module 5 are modulated. In some embodiments, the presents relate to the activation of network module 5, e.g., one or more of (inclusive of all of) XBP1, RGS2, EVL, PSMB8, and HERPUD1.

In some embodiments, one or more genes of network module 6 are modulated. In some embodiments, the presents relate to the activation of network module 6, e.g., one or more of (inclusive of all of) RPS6, ADRB2, GDPD5, APBB2, and MIF.

In some embodiments, one or more genes of network module 7 are modulated. In some embodiments, the presents relate to the activation of network module 7, e.g., one or more of (inclusive of all of) SORBS3, SQSTM1, PGAM1, TWF2, and DRAP1.

In some embodiments, one or more genes of network module 8 are modulated. In some embodiments, the presents relate to the activation of network module 8, e.g., one or more of (inclusive of all of) ZFP36, FOS, ETV1, CCNA1, and HTRA1.

In some embodiments, one or more genes of network module 9 are modulated. In some embodiments, the presents relate to the activation of network module 9, e.g., one or more of (inclusive of all of) DUSP4, GAPDH, RPA3, ADGRG1, and ACOT9.

In some embodiments, one or more genes of network module 10 are modulated. In some embodiments, the presents relate to the activation of network module 10, e.g., one or more of (inclusive of all of) PXN, SLC25A4, DSG2, SATB1, and CALM3.

In some embodiments, one or more genes of network module 11 are modulated. In some embodiments, the presents relate to the activation of network module 11, e.g., one or more of (inclusive of all of) IER3, SOX4, HMOX1, and RHOA.

In some embodiments, one or more genes of network module 12 are modulated. In some embodiments, the presents relate to the activation of network module 12, e.g., one or more of (inclusive of all of) SSBP2, S100A4, ANKRD10, and FCHO1.

In some embodiments, one or more genes of network module 13 are modulated. In some embodiments, the presents relate to the activation of network module 13, e.g., one or more of (inclusive of all of) RPS5, ATP1B1, KDM5B, and SPTAN1.

In some embodiments, one or more genes of network module 14 are modulated. In some embodiments, the presents relate to the activation of network module 14, e.g., one or more of (inclusive of all of) GADD45B, CTSD, HLA-DMA, and FGFR4.

In some embodiments, one or more genes of network module 15 are modulated. In some embodiments, the presents relate to the activation of network module 15, e.g., one or more of (inclusive of all of) SLC1A4, HSPB1, and CDKN2A.

In some embodiments, one or more genes of network module 16 are modulated. In some embodiments, the presents relate to the activation of network module 16, e.g., one or more of (inclusive of all of) STAT3, RAC2, and TIAM1.

In some embodiments, one or more genes of network module 17 are modulated. In some embodiments, the presents relate to the activation of network module 17, e.g., one or more of (inclusive of all of) RALGDS and EZH2.

In some embodiments, the present methods alter a gene signature in the sample of cells, comprising an activation of a network module designated in the network module column of Table 2.

In some embodiments, the activation of the network module designated in the network module column of Table 2 comprises modulating expression and/or activity of 2 or more genes within a network module.

In some embodiments, the activation of the network module designated in the network module column of Table 2 comprises modulating expression and/or activity of all of the genes within a network module.

In some embodiments, the activation of the network module designated in the network module column of Table 2 comprises modulating expression and/or activity of 2 or more genes within 2 or more network modules. In some embodiments, the activation of the network module designated in the network module column of Table 2 comprises modulating expression and/or activity of 2 or more genes (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, or 50 or more genes) within 2 or more network modules (e.g. 2 or more, or 3 or more, or 4 or more, or 5 or more, or 6 or more, or 7 or more, or 8 or more, or 9 or more, or 10 or more, or 11 or more, or 12 or more, or 13 or more network modules).

### Perturbagens

A perturbagen useful in the present disclosure can be a small molecule, a biologic, a protein, a nucleic acid, such as a cDNA over-expressing a wild-type gene or an mRNA encoding a wild-type gene, or any combination of any of the foregoing. Illustrative perturbagens useful in the present disclosure are listed in Table 4.

TABLE 4
Exemplary Perturbagens

| | | Molecular | | |
|---|---|---|---|---|
| Perturbagen | Molecular | weight | | |
| No. | formula | (g/mol) | Dose | |
| 1 | C₁₅H₂₀O₃ | 248.32 | 100 | nM |
| 2 | C₂₃H₃₀ClN₃O | 400 | | |
| 3 | C₂₄H₂₇NO₅S | 441.5 | 10 000 | nM |
| 4 | C₂₀H₃₄AuO₉PS | 678.5 | 100 | nM |
| 5 | C₅H₅N₅ | 135.13 | 10 000 | nM |
| 6 | C₂₄H₂₀Cl₂FN₅O₂ | 500.3 | 100 | nM |
| 7 | C₂₀H₂₁N₅O₆ | 427.4 | 10 000 | nM |
| 8 | C₂₆H₂₉NO | 371.5 | 1000 | nM |
| 9 | C₂₂H₂₇N₃O₄ | 397.5 | 1000 | nM |
| 10 | C₁₄H₁₁NO₅ | 273.24 | 10 000 | nM |
| 11 | C₁₃H₁₈NAO₃ | 278.31 | 1000 | nM |
| 12 | C₂₈H₅₄N₈ | 502.8 | 100 | nM |
| 13 | C₂₁H₂₆N₂O₃ | 354.4 | 10 000 | nM |
| 14 | C₁₉H₂₈O₈ | 384.4 | 10 000 | nM |
| 15 | C₃₁H₂₇IN₄S | 614.5 | 1000 | nM |
| 16 | C₁₉H₂₁N₃O₅ | 371.4 | 10 000 | nM |
| 17 | C₂₄H₄₀O₄ | 392.6 | | |
| 18 | C₁₁H₉FN₆ | 244.23 | 10 000 | nM |
| 19 | C₁₆H₁₄O₃ | 254.28 | 10 000 | nM |
| 20 | C₁₈H₁₄N₄O₂ | 318.3 | | |
| 21 | C₁₇H₂₀N₂S | 284.4 | 1000 | nM |
| 22 | C₂₉H₃ClN₂O₂ | 477 | | |
| 23 | C₂₁H₂₆N₂OS₂ | 386.6 | 1000 | nM |
| 24 | C₃₄H₃₄ClN₂NaO₃S | 609.2 | | |
| 25 | C₃₁H₄₃N₃O₈ | 585.7 | | |
| 26 | C₁₈H₁₈N₂S | 294.4 | 1000 | nM |
| 27 | C₂₄H₃₆O₅ | 404.5 | 1000 | nM |
| 28 | C₂₂H₁₉ClN₄O₅ | 454.9 | 1000 | nM |
| 29 | C₁₂H₉NS | 199.27 | 10 000 | nM |
| 30 | C₂₆H₄₁N₃O₅ | 475.6 | | |
| 31 | C₁₆H₂₄O₄ | 280.36 | | |
| 32 | C₁₉H₂₅BN₄O₄ | 384.2 | | |
| 33 | C₂₀H₁₆N₂O₄ | 348.4 | | |
| 34 | C₂₉H₄₀N₂O₉ | 560.6 | | |
| 35 | C₁₇H₂₂N₂O₃ | 302.37 | | |
| 36 | C₁₅H₁₄N₂O₄S | 318.3 | | |
| 37 | C₂₂H₂₀N₂O₅ | 392.40 | | |
| 38 | C₂₂H₂₃ClN₆O | 422.9 | | |
| 39 | C₂₃H₁₈Cl₃FN₄O₃S | 555.8 | | |
| 40 | C₁₈H₁₈ClN₅O | 355.8 | | |
| 41 | C₃₄H₄₉N₅O₆ | 623.8 | | |
| 42 | C₁₄H₂₀N₂O₃ | 264.32 | | |
| 43 | C₂₁H₂₃N₃O₂ | 349.4 | | |
| 44 | C₂₄H₂₀Cl₂FN₅O | 500.35 | | |

In various embodiments herein, a perturbagen of Table 4 encompasses the perturbagens named. Thus, the named perturbagens of Table 4 represent examples of perturbagens of the present disclosure.

In Table 2, the column titled dose is the concentration of a perturbagen that is capable of increasing gene expression in a T cell, as assayed, at least, by single cell gene expression profiling (GEP).

In embodiments, a perturbagen used in the present disclosure is a variant of a perturbagen of Table 4. A variant may be a derivative, analog, enantiomer or a mixture of enantiomers thereof or a pharmaceutically acceptable salt, solvate, hydrate, co-crystal, clathrate, or polymorph of the perturbagen of Table 4. A variant of a perturbagen of Table 4 retains the biological activity of the perturbagen of Table 4.

### Methods and Perturbagens for Directing a Change in Cell State

In some aspects, the present disclosure is related to an ex-vivo method for directing a change in cell state of a T cell, comprising contacting a population of cells with at least one perturbagen selected from Table 4, or a variant thereof. In this method the perturbagen is capable of altering a gene signature in the T cell.

Another aspect of the present disclosure is related to an ex vivo method for directing a change in cell state of a T cell, comprising contacting a population of cells with at least one perturbagen capable of altering a gene signature in the T cell, wherein altering the gene signature comprises an increase in expression and/or activity in the T cell of one or more genes selected from Table 2 designated as an “up” gene in the gene directionality column of Table 2 and/or a decrease in expression and/or activity in the T cell of one or more genes selected from Table 2 designated as a “down” gene in the gene directionality column of Table 2. In some embodiments, the method is related altering the gene signature which includes an increase in expression and/or activity in the T cell of one or more genes selected from Table 2 designated as an “up” gene in the gene directionality column of Table 2. In some embodiments, altering the gene signature includes a decrease in expression and/or activity in the T cell of one or more genes selected from Table 2 designated as a “down” gene in the gene directionality column of Table 2

In one aspect, the present disclosure is related to an ex vivo method for directing a change in cell state of a T cell. This method includes contacting a population of cells with at least one perturbagen selected from Table 4, or a variant thereof, the perturbagen being capable of altering a gene signature in the T cell, wherein altering the gene signature comprises an increase in expression and/or activity in the T cell of one or more genes selected from Table 2 designated as an “up” gene in the gene directionality column of Table 2 and/or a decrease in expression and/or activity in the T cell of one or more genes selected from Table 2 designated as a “down” gene in the gene directionality column of Table 2. In some embodiments, the method is related altering the gene signature which includes an increase in expression and/or activity in the T cell of one or more genes selected from Table 2 designated as an “up” gene in the gene directionality column of Table 2. In some embodiments, altering the gene signature includes a decrease in expression and/or activity in the T cell of one or more genes selected from Table 2 designated as a “down” gene in the gene directionality column of Table 2

In some embodiments, in the methods described herein, the step of contacting the cell or a population of cells with the perturbagen is performed simultaneously with, or after, genetic manipulations. For example, in the case of Chimeric Antigen Receptor (CAR) T cells, the step of contacting the perturbagen with the CAR-T cells can be performed simultaneously with, or after, genetic manipulations needed to make a CAR-T cell.

In some embodiments, in the methods described herein, the cell or a population of cells being contacted by the perturbagen include a CAR-T cell or a cell that may be used for CAR-T cell based therapy. In various embodiments, the methods described herein prevent exhaustion of the CAR-T cells. In various embodiments, the CAR-T cell therapy comprises CAR-T cells that target antigens (e.g., tumor antigens) such as, but not limited to, carbonic anhydrase IX (CAIX), 5T4, CD19, CD20, CD22, CD30, CD33, CD38, CD47, CS1, CD138, Lewis-Y, L1-CAM, MUC16, ROR-1, IL13Rα2, gp 100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-α), GD2, human epidermal growth factor receptor 2 (HER2), mesothelin, EGFRvIII, fibroblast activation protein (FAP), carcinoembryonic antigen (CEA), and vascular endothelial growth factor receptor 2 (VEGF-R2), as well as other tumor antigens well known in the art. Additional illustrative tumor antigens that may be targeted using the methods described herein, including CAR-T cell therapy, include, but are not limited to MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD37, CD56, CD70, CD74, CD138, AGS16, MUC1, GPNMB, Ep-CAM, PD-L1, and PD-L2. Illustrative CAR T-cell therapy include, but are not limited to, JCAR014 (JUNO THERAPEUTICS), JCAR015 (JUNO THERAPEUTICS), JCAR017 (JUNO THERAPEUTICS), JCAR018 (JUNO THERAPEUTICS), JCAR020 (JUNO THERAPEUTICS), JCAR023 (JUNO THERAPEUTICS), JCAR024 (JUNO THERAPEUTICS), CTL019 (NOVARTIS), KTE-C19 (KITE PHARMA), BPX-401 (BELLICUM PHARMACEUTICALS), BPX-501 (BELLICUM PHARMACEUTICALS), BPX-601 (BELLICUM PHARMACEUTICALS), bb2121 (BLUEBIRD BIO), CD-19 Sleeping Beauty cells (ZIOPHARM ONCOLOGY), UCART19 (CELLECTIS), UCART123 (CELLECTIS), UCART38 (CELLECTIS), UCARTCS1 (CELLECTIS), OXB-302 (OXFORD BIOMEDICA), MB-101 (MUSTANG BIO) and CAR T-cells developed by Innovative Cellular Therapeutics.

In some embodiments, in the methods described herein, the cell or a population of cells being contacted by the perturbagen include at least one tumor infiltrating lymphocytes (TIL). In various embodiments, the methods described herein prevent exhaustion of the TIL.

In some embodiments, in all of the methods described herein-including methods where a population of cells is contacted with a perturbagen—the population of cells can be derived from a subject. In embodiments, in the methods described herein, the population of cells is derived from a subject's bone marrow or a subject's blood. In some embodiments, in the methods described herein, the population of cells is fractionated to obtain fractionated cells prior to contacting with at least one perturbagen. In the embodiments where the population of cells is obtained from the bone marrow of a subject, the subject is treated with or administered one or more mobilization agents. In embodiments, where the population of cells is obtained from blood, the population includes one or more of peripheral blood mononuclear cells (PBMCs), CD3+ T cells, CD3+CD4+ T cells, and CD3+CD8+ T cells.

In some embodiments, the methods described herein include a step of fractionating the population of cells obtained from the subject. In other embodiments, the methods described herein include a step of fractionating as well as enriching the population of cells obtained from the subject. In some embodiments, the population of cells obtained from the subject are fractionated. In embodiments, the fractionated cells are enriched for T cells. In other embodiments, the fractionated cells are enriched for one or more of PBMCs, CD3+ T cells, CD3+CD4+ T cells, and CD3+CD8+ T cells.

In embodiments, the methods described herein further include the step of administering the resultant population of cells to a subject. In embodiments, the resultant population of the cells, as disclosed herein, is a cell population obtained upon contacting the cell population with at least one perturbagen. In other embodiments, the resultant population of the cells is a cell population obtained upon contacting the cell population with at least one perturbagen and having a change/alteration in its gene signature. In some embodiments, the methods described herein are such that the subject is the source of the population of cells to which perturbagen is contacted. In other embodiments, the subject is not the source of the population of cells to which perturbagen is contacted.

In some embodiments, the methods described herein further include the step of contacting the population of cells with one or more of IL-2, an antigen, and an antigen-presenting cell.

In some embodiments, the T cell is selected from an effector T cell, exhausted T cell, and naïve T cell. In some embodiments, the T cells are CD8+ T cells. In other embodiments, the T cells are CD4+ T cells. In some embodiments, in the methods described herein, the cells upon administration to a subject contact a tumor microenvironment, if the subject is afflicted with cancer.

In some embodiments, the methods described herein are related to a change in cell state where the change is the cell state is selected from:
i) prevention or reduction of a cellular transition to an exhausted T cell state;
ii) prevention or reduction of a transition of an effector T cell to an exhausted T cell state;
iii) prevention or reduction of a transition of a naïve T cell to an exhausted T cell state;
iv) stimulation or increase of a transition of an exhausted T cell to an effector T cell;
v) stimulation or increase of a transition of a naïve T cell to an effector T cell;
vi) stimulation or increase of a cell death of an exhausted T cell;
vii) prevention or reduction of a cell death of an effector T cell and/or a naïve T cell; and
viii) increase in the number of one or more of effector T cells and naïve T cells.

In some embodiments, the change in cell state provides an increase in the number of one or more of effector T cells and naïve T cells relative to the number of effector T cells or naïve T cells obtained from a population of T cells that is not contacted with the at least one perturbagen. In other embodiments, the change in cell state provides an increase in the number of one or more of effector T cells and naïve T cells relative to the number of effector T cells or naïve T cells obtained from a population of T cells prior to contacting with the at least one perturbagen.

In some embodiments, in the methods described herein, the change in cell state provides a decrease in the number of one or more of exhausted T cells. In embodiments, the change in cell state provides a decrease in the number of one or more of exhausted T cells relative to the number of exhausted T cells obtained from a population of T cells that is not contacted with the at least one perturbagen. In some embodiments, the change in cell state provides a decrease in the number of one or more of exhausted T cells relative to the number of exhausted T cells obtained from a population of T cells prior to contacting with the at least one perturbagen.

In embodiments, the methods described herein are such that the ratio of the number of effector T cells and/or naïve T cells to the number of exhausted T cells is increased relative to the ratio obtained from a population of T cells that is not contacted with the at least one perturbagen. In other embodiments, the ratio of the number of effector T cells and/or naïve T cells to the number of exhausted T cells is increased relative to the ratio obtained from a population of T cells prior to contacting with the at least one perturbagen.

In some embodiments, the increase in the number of effector T cells and/or naïve T cells is due in part to:
i) increased cell proliferation of the effector T cells and/or naïve T cells;
ii) increased lifespan of the effector T cells and/or naïve T cells; and
iii) reduced cell death among the effector T cells and/or naïve T cells.

In some embodiments, the decrease in the number of exhausted T cells is due in part to decreased cell proliferation of the T cells. In embodiments, the decrease in the number of exhausted T cells is due in part to a decreased lifespan of the T cells. In some embodiments, the decrease in the number of exhausted T cells is due in part to increased cell death among the T cells.

In some embodiments, the number of effector T cells and/or naïve T cells is increased after contacting the population of T cells with the at least one perturbagen. In other embodiments, the number of exhausted T cells is decreased after contacting the population of T cells with the at least one perturbagen.

In some embodiments, in the methods described herein, the ratio of the number of effector T cells and/or naïve T cells to the number of exhausted T cells is increased relative to the ratio obtained from a population of T cells that is not contacted with the at least one perturbagen. In other embodiments, in the methods described herein, the ratio of the number of effector T cells and/or naïve T cells to the number of exhausted T cells is increased relative to the ratio in the population of T cells prior to contacting with the at least one perturbagen.

In some embodiments, the methods described herein promote the formation of a T cells that are responsive to antigen.

In embodiments, the antigen is presented by a professional antigen-presenting cell.

In some embodiments, the methods described herein promote the formation of T cells that are responsive to both TCR engagement and co-stimulation. In other embodiments, the methods described herein promote the formation of T cells that are capable of elaborating cytokines. The cytokines can be, e.g., IL-2, IFNγ, TNFα, and a CC chemokine (or β-chemokine).

In some embodiments, the methods described herein promote the formation of T cells that are capable of proliferating, e.g. in the presence of TCR engagement and co-stimulation. In embodiments, the methods described herein promote the formation of T cells that are anabolic. In other embodiments, the methods described herein promote the formation of T cells that are cytotoxic. In embodiments, the methods described herein promote the formation of effector T cells.

In some embodiments, the methods described herein repress the formation of a T cells that are not responsive or poorly responsive to antigen. In embodiments, the antigen is presented by a professional antigen-presenting cell.

In some embodiments, the methods described herein repress the formation of T cells that are not responsive or poorly responsive to both TCR engagement and co-stimulation. In other embodiments, the methods described herein repress the formation of T cells that are incapable or poorly capable of elaborating cytokines. In embodiments, such cytokines are selected from IL-2, IFNγ, TNFα, and a CC chemokine (or β-chemokine).

In some embodiments, the methods described herein repress the formation of T cells that are incapable or poorly capable of proliferating, e.g. in the presence of signal 1 and signal 2. In order to activate a cytotoxic or helper T cell to proliferate and differentiate into an effector cell, an antigen-presenting cell provides two kinds of signals: signal 1 and signal 2. In some embodiments, signal 1 is provided by a foreign peptide bound to an MHC protein on the surface of the presenting cell. This peptide-MHC complex signals through the T cell receptor and its associated proteins. In some embodiments, signal 2 is provided by costimulatory proteins, e.g., the B7 proteins (CD80 and CD86), which are recognized by the co-receptor protein CD28 on the surface of the T cell. The expression of B7 proteins on an antigen-presenting cell is, e.g., induced by pathogens during the innate response to an infection. Effector T cells act to promote the expression of B7 proteins on antigen-presenting cells, creating a positive feedback loop that amplifies the T cell response. The combined actions of signal 1 and signal 2 stimulate the T cell to proliferate and begin to differentiate into an effector cell.

In some embodiments, the methods described herein repress the formation of T cells that are not anabolic or minimally anabolic. In other embodiments, the methods described herein repress the formation of T cells that are not cytotoxic or minimally cytotoxic. In embodiments, the methods repress the formation of exhausted T cells.

In some embodiments, the methods described herein reduce the number of T cells which demonstrate expression or increased expression of one of more of PD-1, PD-L1, PD-L2, CTLA-4, LAG-3, TIM-3, 2B4/CD244/SLAMF4, CD160, and TIGIT. In embodiments, the methods described herein prevent or reduce formation of T cells which demonstrate expression or increased expression of one of more of PD-1, PD-L1, PD-L2, CTLA-4, LAG-3, TIM-3, 2B4/CD244/SLAMF4, CD160, and TIGIT.

In some embodiments, the exhausted T cells according to the present disclosure demonstrate expression or increased expression of one of more of PD-1, PD-L1, PD-L2, CTLA-4, LAG-3, TIM-3, 2B4/CD244/SLAMF4, CD160, and TIGIT. In some embodiments, the exhausted T cells demonstrate expression or increased expression of a transcription factor selected from TOX, TCF1, NR4A and NFAT. In embodiments, the exhausted T cells demonstrate a loss or reduction of IL-2 productive capacity, proliferative capacity, and/or cytolytic activity. In embodiments, the exhausted T cells demonstrate a loss or reduction of TNFα, IFNγ, and CC chemokine (or β-chemokine) signaling.

In some embodiments, the exhausted T cells demonstrate degranulation and/or increased or high expression of granzyme B. In other embodiments, the exhausted T cells demonstrate poor responsiveness to IL-7 and/or IL-15.

In some embodiments, the methods described herein include contacting cells with at least one perturbagen selected from Table 4, or a variant thereof. In some embodiments, the method includes contacting cells with at least 2, at least 3, at least 4, or at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35, at least 36, at least 37, at least 38, at least 39, at least 40, at least 41, at least 42, or at least 43 perturbagens selected from Table 4, or variants thereof.

In some embodiments, in the methods described herein, the one or more genes are selected from Table 2 designated as an “up” gene in the gene directionality column of Table 2. In some embodiments, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, and 19 or more, 20 or more, 21 or more, 22 or more, 23 or more, 24 or more genes are selected from Table 2. In some embodiments, the one or more genes selected from Table 2 include at least one of MYC, TES, CXCR4, IGFBP3, PRSS23, SYPL1, CYB561, CCNH, XBP1, RPS6, ADRB2, GDPD5, SORBS3, ZFP36, FOS, PXN, SLC25A4, DSG2, SATB1, IER3, SSBP2, RPS5, ATP1B1, and GADD45B.

In some embodiments, in the methods described herein, the one or more genes are selected from Table 2 designated as a “down” gene in the gene directionality column of Table 2. In some embodiments, one or more genes selected from Table 2 designated as a “down” gene in the gene directionality column of Table 2 include 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, and 62 or more genes selected from Table 2. In some embodiments, the one or more genes selected from Table 2 comprises at least one of CDK6, MTHFD2, ID2, SCCPDH, SLC25A46, ETFB, HLA-DRA, CHN1, RAB27A, TBXA2R, NFKB2, ITGAE, SMC4, STMN1, GATA3, ETS1, IQGAP1, CAT, RALA, TSC22D3, CBLB, INPP4B, PLSCR1, NUSAP1, RGS2, EVL, PSMB8, HERPUD1, APBB2, MIF, SQSTM1, PGAM1, TWF2, DRAP1, ETV1, CCNA1, HTRA1, DUSP4, GAPDH, RPA3, ADGRG1, ACOT9, CALM3, SOX4, HMOX1, RHOA, S100A4, ANKRD10, FCHO1, KDM5B, SPTAN1, CTSD, HLA-DMA, FGFR4, SLC1A4, HSPB1, CDKN2A, STAT3, RAC2, TIAM1, RALGDS, and EZH2.

Methods for determining the extension of the lifespan of a specific cell type or a reduction of cell death is well known in the art. As examples, markers for dying cells, e.g., caspases can be detected, or dyes for dead cells, e.g., methylene blue, may be used. Methods for counting cells are well known in the art. Non-limiting examples include hemocytometry, flow cytometry, and cell sorting techniques, e.g., fluorescence activated cell sorting (FACS).

In embodiments, an increase in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (including DMSO).

In various embodiments, an increase in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (including DMSO).

In embodiments, contacting the population of cells comprising a T cell (including, e.g., naïve T cell, effector T cell, and exhausted T cell) occurs in a subject. In embodiments, the subject is a human. In embodiments, the human is an adult human.

In yet another aspect, the present disclosure provides a perturbagen for use in any herein disclosed method. In a further aspect, the present disclosure provides a pharmaceutical composition comprising perturbagen for use in any herein disclosed method.

Embodiments associated with the above aspects are likewise relevant to the present aspect. In other words, each of the embodiments mentioned above for the above aspects may be revised/adapted to be applicable to the present aspect.

### Methods and Perturbagens for Treating a Disease or Disorder

The ability of a perturbagen to mitigate or stop T cell exhaustion would be valuable in designing a therapeutic composition for the treatment of a disease. As an example, for a disease characterized by an increased number of exhausted T cells, a therapeutic composition comprising a perturbagen that decreases the number of exhausted T cells could be beneficial. Similarly, for a disease characterized by an increased number of exhausted T cells, a therapeutic composition comprising a perturbagen that i) increases expression and/or activity in the T cell, ii) prevents or reduces transition of an effector T cell to an exhausted T cell, iii) prevents or reduces transition of a naïve T cell to an exhausted T cell, iv) stimulates or increases transition of an exhausted T cell to an effector T cell, v) stimulates or increases transition of a naïve T cell to an effector T cell, vi) stimulates or increases cell death of an exhausted T cell, vii) prevents or reduces cell death of an effector T cell and/or a naïve T cell, viii) provides an increase in the number of one or more of effector T cells and naïve T cells can be beneficial.

Various aspects of the present disclosure are related to method for treating a disease or disorder characterized by insufficient T cell response, comprising administering to a subject in need thereof a population of cells, the population of cells having been contacted with at least one perturbagen selected from Table 4, or a variant thereof, wherein the at least one perturbagen is capable of changing a gene signature in a T cell. In some embodiments, the method described herein further comprises contacting the population of cells with one or more of IL-2, an antigen, and an antigen-presenting cell. In some embodiments, the disease or disorder is characterized by immune tolerance.

Administering related to this aspect of the disclosure can be done parenterally. In some embodiments, the administering is some via intravenous, intramuscular, intraperitoneal, subcutaneous, intra-articular injection or infusion route. In some embodiments, the administering is intratumoral, for example, in cases where the subject is afflicted with cancer.

In some embodiments, the disease or disorder characterized by insufficient T cell response is a cancer. The cancer, e.g., can be a solid tumor or a liquid tumor. In some embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL;

high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments, the cancer is a melanoma, optionally selected from metastatic melanoma, cutaneous melanoma, BRAF V600, Merkel cell carcinoma, cutaneous squamous cell carcinoma, a lung cancer, optionally selected from NSCLC, metastatic nonsquamous non-small cell lung cancer, metastatic small cell carcinoma, a head and neck cancer, optionally selected from head and neck squamous cell cancer, a bladder cancer, optionally selected from urothelial carcinoma, locally advanced or metastatic urothelial carcinoma, a breast cancer, optionally selected from triple-negative breast cancer), microsatellite Instability-high cancer, gastric cancer, optionally selected from gastric or gastroesophageal junction adenocarcinoma, metastatic colorectal cancer, cervical cancer, optionally selected from recurrent or metastatic cervical cancer, liver cancer, optionally selected from HCC, hematological disorder, optionally selected from primary mediastinal large B-cell lymphoma, and Hodgkin lymphoma.

In some embodiments, the cancer is characterized by a tumor expressing one or more of PD-1, PD-L1, PD-L2, CTLA-4, Tim-3, or LAG-3. In some embodiments, the cancer is poorly responsive or non-responsive to checkpoint inhibitor therapy or has presented as poorly responsive or non-responsive to checkpoint inhibitor therapy. The checkpoint inhibitor therapy is selected from, e.g., an antibody or antibody format specific for one of PD-1, PD-L1, PD-L2, CTLA-4, Tim-3, and LAG-3.

In some embodiments, the antibody or antibody format specific for PD-1 is selected from nivolumab, pembrolizumab, and pidilizumab. In embodiments, the antibody or antibody format specific for PD-L1 is selected from atezolizumab, avelumab, durvalumab, and BMS-936559. In other embodiments, the antibody or antibody format specific for CTLA-4 is selected from ipilimumab (YERVOY), tremelimumab, AGEN1884, and RG2077.

In some embodiments, the tumor is a less-immunogenic tumor and the methods of treatment described herein elicit a potent immune response in the less-immunogenic tumors.

In some embodiments, the tumor has reduced inflammation (“cold tumor”) and the methods of treatment described herein convert the tumor to a responsive, inflamed tumor (“hot tumor”). In embodiments, the cold tumor is characterized by one or more of an absence of T cells, lack of tumor antigens, APC deficit, absence of T cell priming/activation, and impaired trafficking of T cells to the tumor mass. In embodiments, the hot tumor is characterized by one or more of a presence of T cells, presence of tumor antigens, presence of APCs, presence of T cell priming/activation, and effective trafficking of T cells to the tumor mass.

In some embodiments, the method of treatment described herein makes the cancer responsive or more responsive to a checkpoint inhibitor therapy and, optionally one or more chemotherapeutic agents and/or radiotherapy.

In some embodiments, the subject of the methods of treatment described herein is predicted to be poorly responsive or non-responsive to the checkpoint inhibitor therapy based on expression of one or more of PD-1, PD-L1, or PD-L2, in a subject's biological specimen. In other embodiments, the subject is predicted to be poorly responsive or non-responsive to an agent that modulates one or more of PD-1, PD-L1, and PD-L2 based on low on expression of PD-1, PD-L1, and PD-L2 in a tumor specimen. In embodiments, the subject is predicted to be poorly responsive or non-responsive to an agent that modulates one or more of PD-1, PD-L1, and PD-L2 tumor proportion score (TPS) of less than about 49% for PD-L1 staining. The tumor proportion score (TPS) is a PD-L1 measurement which is applied, for example, to lung cancer, head and neck cancer and melanomas. PD-L1 protein expression is determined by using Tumor Proportion Score (TPS), which is the percentage of viable tumor cells showing partial or complete membrane staining.

In some aspects, the present disclosure is related to a method for treating or preventing cancer, comprising administering to a subject in need thereof a population of cells, the population of cells having been contacted with at least one perturbagen selected from Table 4, or a variant thereof, wherein the at least one perturbagen is capable of changing a gene signature in a T cell. In this method, the administering is parenteral, optionally, selected from intravenous, intramuscular, intraperitoneal, subcutaneous, intra-articular injection and infusion. In some embodiments, the administering is intratumoral, for example, in cases where the subject is afflicted with cancer. In some embodiments, in all of the methods described herein-including methods where a population of cells is contacted with a perturbagen—the population of cells can be derived from a subject. In embodiments, in the methods described herein, the population of cells is derived from a subject's bone marrow or a subject's blood. In some embodiments, in the methods described herein, the population of cells is fractionated to obtain fractionated cells prior to contacting with at least one perturbagen. In the embodiments where the population of cells is obtained from the bone marrow of a subject, the subject is treated with or administered one or more mobilization agents. In embodiments, where the population of cells is obtained from blood, the population includes one or more of peripheral blood mononuclear cells (PBMCs), CD3+ T cells, CD3+CD4+ T cells, and CD3+CD8+ T cells.

The cells or population of cells described herein can be obtained from a number of sources, including skin, peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art, may be used. In certain embodiments of the present disclosure, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps.

In some embodiments, the methods described herein include a step of fractionating the population of cells obtained from the subject. In other embodiments, the methods described herein include a step of fractionating as well as enriching the population of cells obtained from the subject. In some embodiments, the population of cells obtained from the subject are fractionated. In embodiments, the fractionated cells are enriched for T cells. In other embodiments, the fractionated cells are enriched for one or more of peripheral blood mononuclear cells (PBMCs), CD3+ T cells, CD3+CD4+ T cells, and CD3+CD8+ T cells. In some embodiments, T cells are isolated from peripheral blood lymphocytes by lysing the red blood cells and depleting the monocytes, for example, by centrifugation through a PERCOLL gradient or by counterflow centrifugal elutriation.

In some embodiments related to this aspect of the disclosure, the cancer is a solid tumor. In other embodiments, related to this aspect of the disclosure, the cancer is a liquid tumor. In some embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments, the cancer is a melanoma, optionally selected from metastatic melanoma, cutaneous melanoma, BRAF V600, Merkel cell carcinoma, cutaneous squamous cell carcinoma, a lung cancer, optionally selected from NSCLC, metastatic nonsquamous non-small cell lung cancer, metastatic small cell carcinoma, a head and neck cancer, optionally selected from head and neck squamous cell cancer, a bladder cancer, optionally selected from urothelial carcinoma, locally advanced or metastatic urothelial carcinoma, a breast cancer, optionally selected from triple-negative breast cancer), microsatellite Instability-high cancer, gastric cancer, optionally selected from gastric or gastroesophageal junction adenocarcinoma, metastatic colorectal cancer, cervical cancer, optionally selected from recurrent or metastatic cervical cancer, liver cancer, optionally selected from HCC, hematological disorder, optionally selected from primary mediastinal large B-cell lymphoma, and Hodgkin lymphoma.

In some embodiments, the cancer is characterized by a tumor expressing one or more of PD-1, PD-L1, PD-L2, CTLA-4, Tim-3, or LAG-3. In some embodiments, the cancer is poorly responsive or non-responsive to checkpoint inhibitor therapy or has presented as poorly responsive or non-responsive to checkpoint inhibitor therapy. The checkpoint inhibitor therapy is selected from, e.g., an antibody or antibody format specific for one of PD-1, PD-L1, PD-L2, CTLA-4, Tim-3, and LAG-3.

In some embodiments, the antibody or antibody format specific for PD-1 is selected from nivolumab, pembrolizumab, and pidilizumab. In embodiments, the antibody or antibody format specific for PD-L1 is selected from atezolizumab, avelumab, durvalumab, and BMS-936559. In other embodiments, the antibody or antibody format specific for CTLA-4 is selected from ipilimumab (YERVOY), tremelimumab, AGEN1884, and RG2077.

In some embodiments, the tumor is a less-immunogenic tumor and the methods of treatment described herein elicit a potent immune response in the less-immunogenic tumors.

In some embodiments, the tumor has reduced inflammation (“cold tumor”) and the methods of treatment described herein convert the tumor to a responsive, inflamed tumor (“hot tumor”). In embodiments, the cold tumor is characterized by one or more of an absence of T cells, lack of tumor antigens, APC deficit, absence of T cell priming/activation, and impaired trafficking of T cells to the tumor mass. In embodiments, the hot tumor is characterized by one or more of a presence of T cells, presence of tumor antigens, presence of APCs, presence of T cell priming/activation and effective trafficking of T cells to the tumor mass.

In some embodiments, the method of treatment described herein makes the cancer responsive or more responsive to a checkpoint inhibitor therapy and, optionally one or more chemotherapeutic agents and/or radiotherapy.

In some embodiments, the subject of the methods of treatment described herein is predicted to be poorly responsive or non-responsive to the checkpoint inhibitor therapy based on expression of one or more of PD-1, PD-L1, or PD-L2, in a subject's biological specimen. In other embodiments, the subject is predicted to be poorly responsive or non-responsive to an agent that modulates one or more of PD-1, PD-L1, and PD-L2 based on low on expression of PD-1, PD-L1, and PD-L2 in a tumor specimen. In embodiments, the subject is predicted to be poorly responsive or non-responsive to an agent that modulates one or more of PD-1, PD-L1, and PD-L2 tumor proportion score (TPS) of less than about 49% for PD-L1 staining. The tumor proportion score (TPS) is a PD-L1 measurement which is applied, for example, to lung cancer, head and neck cancer and melanomas. PD-L1 protein expression is determined by using Tumor Proportion Score (TPS), which is the percentage of viable tumor cells showing partial or complete membrane staining. In some embodiments, the method improves clinical outcome or response to therapy with an anti-cancer agent as compared to clinical outcome or response to therapy in the absence of the perturbagen.

In one aspect, the present disclosure is related to a method for treating or preventing an infection, comprising administering to a subject in need thereof a population of cells, the population of cells having been contacted with at least one perturbagen selected from Table 4, or a variant thereof, wherein the at least one perturbagen is capable of changing a gene signature in a T cell. In some embodiments, the infection is selected from bacterial infections, viral infections, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections. In some embodiments, the method improves clinical outcome or response to therapy with an anti-infective agent or vaccine as compared to clinical outcome or response to therapy in the absence of the perturbagen. In some embodiments, the administering is parenteral, optionally selected from intravenous, intramuscular, intraperitoneal, subcutaneous, intra-articular injection and infusion. In some embodiments, in all of the methods described herein-including methods where a population of cells is contacted with a perturbagen—the population of cells can be derived from a subject. In embodiments, in the methods described herein, the population of cells is derived from a subject's bone marrow or a subject's blood. In some embodiments, in the methods described herein, the population of cells is fractionated to obtain fractionated cells prior to contacting with at least one perturbagen. In the embodiments where the population of cells is obtained from the bone marrow of a subject, the subject is treated with or administered one or more mobilization agents. In embodiments, where the population of cells is obtained from blood, the population includes one or more of peripheral blood mononuclear cells (PBMCs), CD3+ T cells, CD3+CD4+ T cells, and CD3+CD8+ T cells.

In some embodiments, the methods described herein include a step of fractionating the population of cells obtained from the subject. In other embodiments, the methods described herein include a step of fractionating as well as enriching the population of cells obtained from the subject. In some embodiments, the population of cells obtained from the subject are fractionated. In embodiments, the fractionated cells are enriched for T cells. In other embodiments, the fractionated cells are enriched for one or more of PBMCs, CD3+ T cells, CD3+CD4+ T cells, and CD3+CD8+ T cells.

In some embodiments, in the methods described herein, the step of contacting the cell or a population of cells with the perturbagen is performed simultaneously with, or after, genetic manipulations. For example, in the case of Chimeric Antigen Receptor (CAR) T cells, the step of contacting the perturbagen with the CAR-T cells can be performed simultaneously with, or after, genetic manipulations needed to make a CAR-T cell.

In some embodiments, in the methods of treatment described herein, the cell or a population of cells being contacted by the perturbagen include a CAR-T cell or a cell that may be used for CAR-T cell based therapy. In various embodiments, the methods described herein prevent exhaustion of the CAR-T cells. In various embodiments, the CAR-T cell therapy comprises CAR-T cells that target antigens (e.g., tumor antigens) such as, but not limited to, carbonic anhydrase IX (CAIX), 5T4, CD19, CD20, CD22, CD30, CD33, CD38, CD47, CS1, CD138, Lewis-Y, L1-CAM, MUC16, ROR-1, IL 13Rα2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-α), GD2, human epidermal growth factor receptor 2 (HER2), mesothelin, EGFRvIII, fibroblast activation protein (FAP), carcinoembryonic antigen (CEA), and vascular endothelial growth factor receptor 2 (VEGF-R2), as well as other tumor antigens well known in the art. Additional illustrative tumor antigens that may be targeted using the methods described herein, including CAR-T cell therapy, include, but are not limited to MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD37, CD56, CD70, CD74, CD138, AGS16, MUC1, GPNMB, Ep-CAM, PD-L1, and PD-L2. Illustrative CAR T-cell therapy include, but are not limited to, JCAR014 (JUNO THERAPEUTICS), JCAR015 (JUNO THERAPEUTICS), JCAR017 (JUNO THERAPEUTICS), JCAR018 (JUNO THERAPEUTICS), JCAR020 (JUNO THERAPEUTICS), JCAR023 (JUNO THERAPEUTICS), JCAR024 (JUNO THERAPEUTICS), CTL019 (NOVARTIS), KTE-C19 (KITE PHARMA), BPX-401 (BELLICUM PHARMACEUTICALS), BPX-501 (BELLICUM PHARMACEUTICALS), BPX-601 (BELLICUM PHARMACEUTICALS), bb2121 (BLUEBIRD BIO), CD-19 Sleeping Beauty cells (ZIOPHARM ONCOLOGY), UCART19 (CELLECTIS), UCART123 (CELLECTIS), UCART38 (CELLECTIS), UCARTCS1 (CELLECTIS), OXB-302 (OXFORD BIOMEDICA), MB-101 (MUSTANG BIO) and CAR T-cells developed by Innovative Cellular Therapeutics.

In some embodiments, in the methods described herein, the cell or a population of cells being contacted by the perturbagen include at least one tumor infiltrating lymphocytes (TIL). In various embodiments, the methods described herein prevent exhaustion of the TIL.

In some aspects, the present disclosure is related to a method for making a therapeutic agent for a cancer or infection.

This method includes the steps of (a) identifying a therapeutic agent for therapy using methods described herein and (b) formulating the therapeutic agent for the treatment of the disease or disorder.

In some embodiments, the method includes making a therapeutic agent for cancer. In embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments, the method includes making a therapeutic agent for an infection, an infectious disease or disorder. In embodiments, the infection is selected from bacterial infections, viral infections, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

In embodiments, the administering the cell is via intravenous injection or intravenous infusion. In other embodiments, the administering of the cell is via intravenous injection or intravenous infusion. In some embodiments, the administering is simultaneously or sequentially to one or more mobilization agents.

In some embodiments, the methods described herein are useful for treatment of a disease or disorder characterized by an abnormal number of exhausted T cells.

In some embodiments, the methods described herein are where at least one perturbagen is contacted to a subject's cell on the basis of previously determining the subject exhibits an abnormal number of exhausted T cells or a disease or disorder characterized thereby.

In embodiments related to this aspect of the disclosure, the administering is directed to the bone marrow of the subject. In embodiments, the administering the cell is via intravenous injection or intravenous infusion. In embodiments, the administering is simultaneously or sequentially to one or more mobilization agents. In embodiments, the administering occurs about once per day for one or more days. In embodiments, the administering occurs more than once per day for one or more days. In embodiments, the administering occurs at most once per day for one or more days. In embodiments, the administering occurs substantially continuously per administration period.

### Administration, Dosing, and Treatment Regimens

As examples, administration results in the delivery of one or more cells or a population of cells disclosed herein into the bloodstream (via enteral or parenteral administration), or alternatively, the one or more cells or a population of cells is administered directly to the site of T cell development, proliferation and/or maturation, i.e., in the bone marrow or in thymus. In some embodiments, the administering is done parenterally. In embodiments, the administering is done via intravenous, intramuscular, intraperitoneal, subcutaneous, intra-articular injection, or infusion route. In embodiments, the administration is done intratumorally, if the subject is suffering from a cancer.

In some embodiments, a subject—as disclosed herein—is treated with or administered a mobilization agent before a cell or a population of cells is obtained from the subject. Delivery of the mobilization agent can be done parenterally.

In some embodiments, the mobilization agent is delivered into the bloodstream via intravenous injection or intravenous infusion and may precede or be contemporaneous with stem cell mobilization. In stem cell mobilization, certain drugs are used to cause the movement of stem cells from the bone marrow into the bloodstream. In some embodiments, once the mobilization agent is in the bloodstream, cells or a population of cells are isolated from the subject and contacted with the one or more perturbagens that are able to alter a gene signature in, e.g., a T cell. Drugs and methods relevant to stem cell mobilization are well known in the art; see, e.g., Mohammadi et al., “Optimizing Stem Cells Mobilization Strategies to Ameliorate Subject Outcomes: A Review of Guide-lines and Recommendations.” Int. J. Hematol. Oncol. Stem Cell Res. 2017 Jan. 1; 11 (1): 78-88; Hopman and DiPersio, “Advances in Stem Cell Mobilization.” Blood Review, 2014, 28 (1): 31-40; and Kim “Hematopoietic Stem Cell Mobilization: Current Status and Future Perspective.” Blood Res. 2017 June; 52 (2): 79-81, the content of each of which is incorporated herein by reference in its entirety.

Dosage forms suitable for parenteral administration include, for example, solutions, suspensions, dispersions, emulsions, and the like. They may also be manufactured in the form of sterile solid compositions that can be dissolved or suspended in sterile injectable medium immediately before use. They may contain, for example, suspending or dispersing agents known in the art.

The dosage of a cell or a population of cell disclosed herein as well as the dosing schedule can depend on various parameters and factors, including, but not limited to, the cell type, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the cells being administered, the time of administration, the route of administration, the nature of the formulation, the rate of excretion, rate of cell death, the particular disease being treated, the severity of the disorder, and the anatomical location of the disorder. Some variations in the dosage can be expected.

A mobilization agent disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds. In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105). In another embodiment, a controlled-release system can be placed in proximity of the target area to be treated, e.g., the bone marrow, thus requiring only a fraction of the systemic dose (see, e.g., Goodson, in Medical Applications of Controlled Release, supra, vol. 2, pp. 115-138 (1984). Other controlled-release systems discussed in the review by Langer, 1990, Science 249:1527-1533 may be used.

The dosage regimen utilizing any cell or population of cells disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific cell type employed. Any cell or a population of cells disclosed herein can be administered in a single daily dose, or the total daily dosage can be administered in divided doses of two, three or four times daily. Furthermore, any cell or cell type disclosed herein can be administered continuously rather than intermittently throughout the dosage regimen.

### Pharmaceutical Compositions and Formulations

Aspects of the present disclosure include a pharmaceutical composition comprising a therapeutically effective amount of cells (e.g. T cells, CAR-T cells, TILs), or populations of cells, and/or mobilization agents as disclosed herein.

The cells or a population of cells, as described herein, may be administered either alone, or as a pharmaceutical composition in combination with diluents and/or with other components such as IL-2 or other cytokines or cell populations. Briefly, pharmaceutical compositions of the present disclosure may comprise a target cell or cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in one aspect formulated for intravenous administration.

Pharmaceutical compositions of the present disclosure may be administered in a manner appropriate to the disease to be treated (or prevented). The quantity and frequency of administration will be determined by such factors as the condition of the subject, and the type and severity of the subject's disease, although appropriate dosages may be determined by clinical trials.

The pharmaceutical compositions comprising the cells or population of cells of the present disclosure may conveniently be presented in unit dosage forms and may be prepared by any of the methods well known in the art of pharmacy. Such methods generally include the step of bringing the cells or population of cells disclosed herein into association with a carrier, which constitutes one or more accessory ingredients. Typically, the pharmaceutical compositions are prepared by uniformly and intimately bringing the cells or population of cells disclosed herein into association with a liquid carrier, a finely divided solid carrier, or both, and then, if necessary, shaping the product into dosage forms of the desired formulation.

The precise amount of the compositions of the present disclosure to be administered can be determined by a physician with consideration of individual differences in age, weight, tumor size, extent of infection or metastasis, and condition of the subject. It can generally be stated that a pharmaceutical composition comprising the cells or population of cells described herein may be administered at a dosage of 10⁴ to 10⁹ cells, in some instances 10⁵ to 10⁶ cells, including all integer values within those ranges. The cell compositions may also be administered multiple times at these dosages. The cells can be administered by using infusion techniques that are commonly known in immunotherapy (see, e.g., Rosenberg et al., New Eng. J. of Med. 319: 1676, 1988, which is hereby incorporated by reference in its entirety). The optimal dosage and treatment regime for a particular subject can readily be determined by one skilled in the art of medicine by monitoring the subject for signs of disease and adjusting the treatment accordingly.

Administration of the cells of the disclosure may be carried out using any convenient means, including by aerosol inhalation, injection, ingestion, transfusion, implantation or transplantation. The compositions described herein may be administered to a subject transarterially, subcutaneously, intradermally, intratumorally, intranodally, intramedullary, intramuscularly, by intravenous (i.v.) injection, or intraperitoneally. In one embodiment, the cell compositions of the present disclosure are administered to a subject by intradermal or subcutaneous injection. In another embodiment, the cell compositions of the present disclosure are administered by i.v. injection. The compositions of cells may be injected directly into a tumor, lymph node, or site of infection.

In certain embodiments of the present disclosure, cells related to the methods described herein, or other methods known in the art, are administered to a subject in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS subjects or efalizumab treatment for psoriasis subjects or other treatments for PML subjects.

In further embodiments, the cells of the disclosure may be used in combination with chemotherapy, radiation, immunosuppressive agents, other immunoablative agents, antibody therapies, or irradiation. In a further embodiment, the cell compositions of the present disclosure are administered to a subject in conjunction with (e.g., before, simultaneously or following) bone marrow transplantation or T cell ablative therapy using, e.g., chemotherapy agents. For example, in one embodiment, subjects may undergo standard treatment with high dose chemotherapy followed by peripheral blood stem cell transplantation. In certain embodiments, following the transplant, subjects receive an infusion of the cells of the present disclosure. In an additional embodiment, the cells of the present disclosure are administered before or following surgery.

The dosage of the above treatments to be administered to a subject will vary with the precise nature of the condition being treated and the recipient of the treatment. The scaling of dosages for human administration can be performed according to art-accepted practices.

Another aspect of the present disclosure includes a pharmaceutical composition comprising one or more perturbagens where the pharmaceutical composition is contacted with the cells (e.g. T cells, CAR-T cells, TILs) or population of cells disclosed herein in, e.g., an ex vivo setting. The amount of perturbagens included in the composition for contacting the cells or population of cell ex vivo is—in some embodiments-suitable for or capable of directing a change in cell state of a T cell. In some embodiments, the amount of perturbagens included in the composition for contacting the cells or population of cell ex vivo is—in some embodiments-capable of altering a gene signature in the T cell. The perturbagens disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. In embodiments, the compositions disclosed herein are in the form of a pharmaceutically acceptable salt.

Further, any perturbagen disclosed herein can be administered ex vivo to a cell or a population of cells disclosed herein as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any perturbagen disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference. In embodiments, the compositions, e.g., pharmaceutical compositions, disclosed herein are suspended in a saline buffer (including, without limitation TBS, PBS, and the like).

The mobilization agents, and/or perturbagens disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Where necessary, the pharmaceutical compositions comprising the perturbagens or mobilization agents can also include a solubilizing agent. Also, the agents can be delivered with a suitable vehicle or delivery device as known in the art. Combination therapies, comprising more than one perturbagen, can be co-delivered in a single delivery vehicle or delivery device.

Compositions for administration can optionally include a local anesthetic such as, for example, lignocaine to lessen pain at the site of the injection.

In embodiments, any cells, population of cells and/or mobilization agents disclosed herein are formulated in accordance with routine procedures as a pharmaceutical composition adapted for a mode of administration disclosed herein.

### Other Aspects of the Present Disclosure

Embodiments associated with any of the above-disclosed aspects are likewise relevant to the below-mentioned aspects. In other words, each of the embodiments mentioned above for the above aspects may be revised/adapted to be applicable to the below aspects.

In some embodiments, the methods of treatments described herein include a step of selecting a subject. In such embodiments, the subject is selected by steps including: obtaining from the subject a sample of cells comprising a T cell; and contacting the sample of cells with least one perturbagen selected from Table 4, or a variant thereof, wherein the at least one perturbagen alters a gene signature in the sample of cells.

In some embodiments, the methods of treatments described herein include a step of selecting a subject. In such embodiments, the subject is selected by steps including: obtaining from the subject a sample of cells comprising a T cell; and contacting the sample of cells with at least one perturbagen capable of altering a gene signature in a T cell, wherein the at least one perturbagen increases in the sample of cells the expression and/or activity of one or more genes selected from Table 2 designated as an “up” gene in the gene directionality column of Table 2 and/or decreases in the sample of cells the expression and/or activity of one or more genes selected from Table 2 designated as a “down” gene in the gene directionality column of Table 2. In embodiments, the at least one perturbagen increases in the sample of cells the expression and/or activity of one or more genes selected from Table 2 designated as an “up” gene in the gene directionality column of Table 2 In embodiments, the at least one perturbagen decreases in the sample of cells the expression and/or activity of one or more genes selected from Table 2 designated as a “down” gene in the gene directionality column of Table 2 In some embodiments, in the methods described herein which include a step of selecting a subject, the population of cells is derived from the subject. In other embodiments, the population of cells is not derived from the subject. In embodiments, the population of cells is derived from the subject's bone marrow or the subject's blood.

In some embodiment, where the methods include a step of selecting a subject, the population of cells is fractionated prior to contacting with at least one perturbagen. Moreover, in some embodiments, the fractionated cells are enriched for T cells.

Yet another aspect of the present disclosure is a use of the perturbagen of Table 4, or a variant thereof in the manufacture of a medicament for treating a disease or disorder characterized by production of exhausted T cells, an increased rate of production of exhausted T cells, an increased rate of production of exhausted T cells, an abnormal number of exhausted T cells, or a high number of exhausted T cells.

In some aspects, the present disclosure is related to a method of identifying a candidate perturbation for reducing or reversing the conversion of a T cell into an exhausted T cell. This method includes i) exposing a starting population of T cells to a perturbation; ii) identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of T cells into exhausted T cells following exposure of the population of cells to the perturbation; and iii) identifying the perturbation as a candidate perturbation for reducing or reversing the conversion of a T cell thereof based on the perturbation signature, wherein the perturbation signature is an increase in expression and/or activity in the T cell of one or more genes selected from Table 2 designated as an “up” gene in the gene directionality column of Table 2 and/or decreases in the sample of cells the expression and/or activity of one or more genes selected from Table 2 designated as a “down” gene in the gene directionality column of Table 2.

In some embodiments, the perturbation signature is an increase in expression and/or activity in the T cell of one or more genes selected from Table 2 designated as an “up” gene in the gene directionality column of Table 2.

In embodiments, the perturbation signature is a decrease in expression and/or activity in the T cell of one or more genes selected from Table 2 designated as a “down” gene in the gene directionality column of Table 2

Yet another aspect of the present disclosure is a perturbagen capable of causing a change in a gene signature within naïve T cells, effector T cells or exhausted T cells. In an aspect, the present disclosure provides a perturbagen capable of causing a change in a cell fate of T cells. In another aspect, the present disclosure provides a perturbagen capable of causing a change in a gene signature and a change in a cell fate of T cells.

In yet another aspect, the present disclosure provides a pharmaceutical composition comprising any herein disclosed perturbagen. In a further aspect, the present disclosure provides a unit dosage form comprising an effective amount of the pharmaceutical composition comprising any herein disclosed perturbagen.

The instant disclosure also provides certain embodiments as follows:

Embodiment 140: An ex-vivo method for directing a change in cell state of a T cell, comprising: contacting a population of cells with at least one perturbagen selected from Table 4, or a variant thereof, the perturbagen being capable of altering a gene signature in the T cell.

Embodiment 141: An ex-vivo method for directing a change in cell state of a T cell, comprising: contacting a population of cells with at least one perturbagen capable of altering a gene signature in the T cell, wherein altering the gene signature comprises an increase in expression and/or activity in the T cell of one or more genes selected from Table 2 designated as an “up” gene in the gene directionality column of Table 2 and/or a decrease in expression and/or activity in the T cell of one or more genes selected from Table 2 designated as a “down” gene in the gene directionality column of Table 2.

Embodiment 142: An ex-vivo method for directing a change in cell state of a T cell, comprising: contacting a population of cells with at least one perturbagen selected from Table 4, or a variant thereof, the perturbagen being capable of altering a gene signature in the T cell, wherein altering the gene signature comprises an increase in expression and/or activity in the T cell of one or more genes selected from Table 2 designated as an “up” gene in the gene directionality column of Table 2 and/or a decrease in expression and/or activity in the T cell of one or more genes selected from Table 2 designated as a “down” gene in the gene directionality column of Table 2.

Embodiment 143: The method of Embodiments 140-142, wherein the population of cells is derived from a subject, optionally, from the subject's bone marrow or the subject's blood.

Embodiment 144: The method of Embodiments 140-143, wherein the population of cells is fractionated to obtain fractionated cells prior to contacting with at least one perturbagen, optionally, wherein the fractionated cells are enriched for T cells.

Embodiment 145: The method of Embodiments 140-144, wherein the method further comprises the step of administering a resultant population of cells to a subject wherein resultant population of cells is obtained upon contacting the population of cells with at least one perturbagen, optionally, wherein the subject is the source of the population of cells to which perturbagen is contacted or the subject is not the source of the population of cells to which perturbagen is contacted.

Embodiment 146: The method of Embodiments 140-145, wherein the method further comprises contacting the population of cells with one or more of IL-2, an antigen, and an antigen-presenting cell.

Embodiment 147: The method of Embodiment 141 or 142, wherein altering the gene signature comprises an activation of a network module designated in the network module column of Table 2.

Embodiment 148: The method of Embodiment 147, wherein the activation of the network module comprises modulating expression and/or activity of 2 or more genes within a network module.

Embodiment 149: The method of Embodiment 148, wherein the activation of the network module comprises modulating expression and/or activity of all genes within a network module.

Embodiment 150: The method of Embodiment 149, wherein the activation of the network module comprises modulating expression and/or activity of 2 or more genes within 2 or more network modules.

Embodiment 151: The method of any one of Embodiments 140-150, wherein the T cell is selected from an effector T cell, an exhausted T cell, and a naïve T cell.

Embodiment 152: The method of any one of Embodiments 140-151, wherein the change in cell state is a prevention or reduction of a cellular transition to an exhausted T cell state.

Embodiment 153: The method of any one of Embodiments 140-151, wherein the change in cell state is a prevention or reduction of a transition of an effector T cell to an exhausted T cell state.

Embodiment 154: The method of any one of Embodiments 140-151, wherein the change in cell state is a prevention or reduction of a transition of a naïve T cell to an exhausted T cell state.

Embodiment 155: The method of any one of Embodiments 140-151, wherein the change in cell state is a stimulation or increase of a transition of an exhausted T cell to an effector T cell.

Embodiment 156: The method of any one of Embodiments 140-151, wherein the change in cell state is a stimulation or increase of a transition of a naïve T cell to an effector T cell.

Embodiment 157: The method of any one of Embodiments 140-151, wherein the change in cell state is a stimulation or increase of a cell death of an exhausted T cell.

Embodiment 158: The method of any one of Embodiments 140-151, wherein the change in cell state is a prevention or reduction of a cell death of an effector T cell and/or a naïve T cell.

Embodiment 159: The method of any one of Embodiments 140-151, wherein the change in cell state provides an increase in the number of one or more of effector T cells and naïve T cells.

Embodiment 160: The method of any one of Embodiments 140-151, wherein the change in cell state provides an increase in the number of one or more of effector T cells and naïve T cells relative to the number of effector T cells or naïve T cells obtained from a population of T cells that is not contacted with the at least one perturbagen.

Embodiment 161: The method of any one of Embodiments 140-151, wherein the change in cell state provides an increase in the number of one or more of effector T cells and naïve T cells relative to the number of effector T cells or naïve T cells obtained from a population of T cells prior to contacting with the at least one perturbagen.

Embodiment 162: The method of any one of Embodiments 140-151, wherein the change in cell state provides a decrease in the number of one or more of exhausted T cells.

Embodiment 163: The method of any one of Embodiments 140-151, wherein the change in cell state provides a decrease in the number of one or more of exhausted T cells relative to the number of exhausted T cells obtained from a population of T cells that is not contacted with the at least one perturbagen.

Embodiment 164: The method of any one of Embodiments 140-151, wherein the change in cell state provides a decrease in the number of one or more of exhausted T cells relative to the number of exhausted T cells obtained from a population of T cells prior to contacting with the at least one perturbagen.

Embodiment 165: The method of any one of Embodiments 140-151, wherein the ratio of the number of effector T cells and/or naïve T cells to the number of exhausted T cells is increased relative to the ratio obtained from a population of T cells that is not contacted with the at least one perturbagen.

Embodiment 166: The method of any one of Embodiments 140-151, wherein the ratio of the number of effector T cells and/or naïve T cells to the number of exhausted T cells is increased relative to the ratio obtained from a population of T cells prior to contacting with the at least one perturbagen.

Embodiment 167: The method of any one of Embodiments 159-161, wherein the increase in the number of effector T cells and/or naïve T cells is due in part to increased cell proliferation of the effector T cells and/or naïve T cells.

Embodiment 168: The method of any one of Embodiments 159-161, wherein the increase in the number of effector T cells and/or naïve T cells is due in part to an increased lifespan of the effector T cells and/or naïve T cells.

Embodiment 169: The method of any one of Embodiments 159-161, wherein the increase in the number of effector T cells and/or naïve T cells is due in part to reduced cell death among the effector T cells and/or naïve T cells.

Embodiment 170: The method of any one of Embodiments 162-164, wherein the decrease in the number of exhausted T cells is due in part to decreased cell proliferation of the T cells.

Embodiment 171: The method of any one of Embodiments 162-164, wherein the decrease in the number of exhausted T cells is due in part to a decreased lifespan of the T cells.

Embodiment 172: The method of any one of Embodiments 162-164, wherein the decrease in the number of exhausted T cells is due in part to increased cell death among the T cells.

Embodiment 173: The method of any one of Embodiments 159-161, wherein the number of effector T cells and/or naïve T cells is increased after contacting the population of T cells with the at least one perturbagen.

Embodiment 174: The method of any one of Embodiments 162-164, wherein the number of exhausted T cells is decreased after contacting the population of T cells with the at least one perturbagen.

Embodiment 175: The method of any one of Embodiments 140-174, wherein the method promotes the formation of a T cells that are responsive to antigen, the antigen optionally being presented by a professional antigen-presenting cell.

Embodiment 176: The method of any one of Embodiments 140-175, wherein the method promotes the formation of T cells that are responsive to both TCR engagement and co-stimulation.

Embodiment 177: The method of any one of Embodiments 140-176, wherein the method promotes the formation of T cells that are capable of elaborating cytokines.

Embodiment 178: The method of Embodiment 177, wherein the cytokines are selected from IL-2, IFNγ, TNFα, and a CC chemokine (or β-chemokine).

Embodiment 179: The method of any one of Embodiments 140-178, wherein the method promotes the formation of T cells that are capable of proliferating, e.g., in the presence of TCR engagement and co-stimulation.

Embodiment 180: The method of any one of Embodiments 140-179, wherein the method promotes the formation of T cells that are anabolic.

Embodiment 181: The method of any one of Embodiments 140-179, wherein the method promotes the formation of T cells that are cytotoxic.

Embodiment 182: The method of any one of Embodiments 140-179, wherein the method promotes the formation of effector T cells.

Embodiment 183: The method of any one of Embodiments 140-179, wherein the method represses the formation of a T cells that are not responsive or poorly responsive to antigen.

Embodiment 184: The method of Embodiment 183, wherein the antigen is being presented by a professional antigen-presenting cell.

Embodiment 185: The method of any one of Embodiments 140-178, wherein the method represses the formation of T cells that are not responsive or poorly responsive to both TCR engagement and co-stimulation.

Embodiment 186: The method of any one of Embodiments 140-178, wherein the method represses the formation of T cells that of a T cells that are incapable or poorly capable of elaborating cytokines.

Embodiment 187: The method of Embodiment 186, wherein the cytokines are selected from IL-2, IFNγ, TNFα, and a CC chemokine (or β-chemokine).

Embodiment 188: The method of any one of Embodiments 140-178, wherein the method represses the formation of T cells that are incapable or poorly capable of proliferating, e.g. in the presence of signal 1 and signal 2.

Embodiment 189: The method of Embodiment 188, wherein the method represses the formation of T cells that are not anabolic or minimally anabolic.

Embodiment 190: The method of any one of Embodiments 140-178, wherein the method represses the formation of T cells that are not cytotoxic or minimally cytotoxic.

Embodiment 191: The method of any one of Embodiments 140-190, wherein the method represses the formation of exhausted T cells.

Embodiment 192: The method of any one of Embodiments 140-191, wherein the method reduces the number of T cells which demonstrate expression or increased expression of one of more of PD-1, PD-L1, PD-L2, CTLA-4, LAG-3, TIM-3, 2B4/CD244/SLAMF4, CD160, and TIGIT.

Embodiment 193: The method of any one of Embodiments 140-191, wherein the method prevents or reduces formation of T cells which demonstrate expression or increased expression of one of more of PD-1, PD-L1, PD-L2, CTLA-4, LAG-3, TIM-3, 2B4/CD244/SLAMF4, CD160, and TIGIT.

Embodiment 194: The method of Embodiment 191, wherein the exhausted T cells demonstrate expression or increased expression of one of more of PD-1, PD-L1, PD-L2, CTLA-4, LAG-3, TIM-3, 2B4/CD244/SLAMF4, CD160, and TIGIT.

Embodiment 195: The method of Embodiment 191, wherein the exhausted T cells demonstrate expression or increased expression of a transcription factor selected from TOX, TCF1, NR4A, and NFAT.

Embodiment 196: The method of Embodiment 191, wherein the exhausted T cells demonstrate a loss or reduction of IL-2 productive capacity, proliferative capacity, and/or cytolytic activity.

Embodiment 197: The method of Embodiment 191, wherein the exhausted T cells demonstrate a loss or reduction of TNFα, IFNγ, and CC chemokine (or β-chemokine) signaling.

Embodiment 198: The method of Embodiment 191, wherein the exhausted T cells demonstrate degranulation and/or increased or high expression of granzyme B.

Embodiment 199: The method of Embodiment 191, wherein the exhausted T cells demonstrate poor responsiveness to IL-7.

Embodiment 200: The method according to Embodiment 191, wherein the exhausted T cells demonstrate poor responsiveness to IL-15

Embodiment 201: The method of any of Embodiments 140-200, wherein the T cells are CD8+ T cells.

Embodiment 202: The method of any of Embodiments 140-201, wherein the T cells are CD4+ T cells.

Embodiment 203: The method of any of Embodiments 140, 143-146, or 151-202, wherein the at least one perturbagen selected from Table 4, or a variant thereof, comprises at least 2, at least 3, at least 4, or at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35, at least 36, at least 37, at least 38, at least 39, at least 40, at least 41, at least 42, or at least 43 perturbagens selected from Table 4, or variants thereof.

Embodiment 204: The method of any of Embodiments 141-203, wherein the one or more genes selected from Table 2 designated as an “up” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, and 19 or more, 20 or more, 21 or more, 22 or more, 23 or more, 24 or more genes selected from Table 2 designated as an “up” gene in the gene directionality column of Table 2.

Embodiment 205: The method of Embodiment 204, wherein the one or more genes selected from Table 2 comprises at least one of MYC, TES, CXCR4, IGFBP3, PRSS23, SYPL1, CYB561, CCNH, XBP1, RPS6, ADRB2, GDPD5, SORBS3, ZFP36, FOS, PXN, SLC25A4, DSG2, SATB1, IER3, SSBP2, RPS5, ATP1B1, and GADD45B.

Embodiment 206: The method of Embodiments 141-205, wherein the one or more genes selected from Table 2 designated as a “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, and 62 or more genes selected from Table 2 designated as a “down” gene in the gene directionality column of Table 2.

Embodiment 207: The method of Embodiment 206 wherein the one or more genes selected from Table 2 comprises at least one of CDK6, MTHFD2, ID2, SCCPDH, SLC25A46, ETFB, HLA-DRA, CHN1, RAB27A, TBXA2R, NFKB2, ITGAE, SMC4, STMN1, GATA3, ETS1, IQGAP1, CAT, RALA, TSC22D3, CBLB, INPP4B, PLSCR1, NUSAP1, RGS2, EVL, PSMB8, HERPUD1, APBB2, MIF, SQSTM1, PGAM1, TWF2, DRAP1, ETV1, CCNA1, HTRA1, DUSP4, GAPDH, RPA3, ADGRG1, ACOT9, CALM3, SOX4, HMOX1, RHOA, S100A4, ANKRD10, FCHO1, KDM5B, SPTAN1, CTSD, HLA-DMA, FGFR4, SLC1A4, HSPB1, CDKN2A, STAT3, RAC2, TIAM1, RALGDS, and EZH2.

Embodiment 208: The method of any of Embodiments 140-207, wherein the subject is a human.

Embodiment 209: The method of Embodiment 208, wherein the human is an adult human.

Embodiment 210: The method of any of Embodiments 140-209, wherein the administering is parenteral.

Embodiment 211: The method of Embodiment 210, wherein the administering is done via intravenous, intramuscular, intraperitoneal, subcutaneous, intra-articular injection, or infusion route.

Embodiment 212: The method of Embodiment 210, wherein the administering is done intravenously.

Embodiment 213: The method of Embodiment 210, wherein the administering is via infusion.

Embodiment 214: The method of Embodiment 210, wherein the administering is intratumoral, if the subject is afflicted with a cancer.

Embodiment 215: The method of any of Embodiments 140-214, wherein the cells upon administration to a subject contact the tumor microenvironment, if the subject is afflicted with a cancer.

Embodiment 216: A perturbagen for use in the method of any of Embodiments 140-215.

Embodiment 217: A pharmaceutical composition comprising the perturbagen of Embodiment 216.

Embodiment 218: A method for treating a disease or disorder characterized by insufficient T cell response, comprising administering to a subject in need thereof a population of cells, the population of cells having been contacted with at least one perturbagen selected from Table 4, or a variant thereof, wherein the at least one perturbagen is capable of changing a gene signature in a T cell.

Embodiment 219: The method of Embodiment 218, wherein the disease or disorder is characterized by immune tolerance.

Embodiment 220: The method of any one of Embodiments 218 or 219, wherein the administering is parenteral.

Embodiment 221: The method of Embodiment 220, wherein the administering is done via an intravenous, intramuscular, intraperitoneal, subcutaneous, intra-articular injection, or infusion route.

Embodiment 222: The method of Embodiment 220, wherein the administering is intratumoral, if the subject is afflicted with cancer.

Embodiment 223: The method of any one of Embodiments 218-222, wherein the method further comprises contacting the population of cells with one or more of IL-2, an antigen, and an antigen-presenting cell.

Embodiment 224: The method of any one of Embodiments 218-223, wherein the disease or disorder characterized by insufficient T cell response is a cancer.

Embodiment 225: The method of Embodiment 224, wherein the cancer is a solid tumor.

Embodiment 226: The method of Embodiment 224, wherein the cancer is a liquid tumor.

Embodiment 227: The method of one of Embodiments 224-226, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

Embodiment 228: The method of one of Embodiments 224-227, wherein the cancer is a melanoma, optionally selected from metastatic melanoma, cutaneous melanoma, BRAF V600, Merkel cell carcinoma, cutaneous squamous cell carcinoma, a lung cancer, optionally selected from NSCLC, metastatic nonsquamous non-small cell lung cancer, metastatic small cell carcinoma, a head and neck cancer, optionally selected from head and neck squamous cell cancer, a bladder cancer, optionally selected from urothelial carcinoma, locally advanced or metastatic urothelial carcinoma, a breast cancer, optionally selected from triple-negative breast cancer), microsatellite Instability-high cancer, gastric cancer, optionally selected from gastric or gastroesophageal junction adenocarcinoma, metastatic colorectal cancer, cervical cancer, optionally selected from recurrent or metastatic cervical cancer, liver cancer, optionally selected from HCC, hematological disorder, optionally selected from primary mediastinal large B-cell lymphoma, and Hodgkin lymphoma.

Embodiment 229: The method of one of Embodiments 224-228, wherein the cancer is characterized by a tumor expressing one or more of PD-1, PD-L1, PD-L2, CTLA-4, Tim-3, or LAG-3.

Embodiment 230: The method of one of Embodiments 224-229, wherein the cancer is poorly responsive or non-responsive to checkpoint inhibitor therapy or has presented as poorly responsive or non-responsive to checkpoint inhibitor therapy.

Embodiment 231: The method of Embodiment 230, wherein the checkpoint inhibitor therapy is selected from an antibody or antibody format specific for one of PD-1, PD-L1, PD-L2, CTLA-4, Tim-3, and LAG-3.

Embodiment 232: The method of Embodiment 231, wherein the antibody or antibody format specific for PD-1 is selected from nivolumab, pembrolizumab, and pidilizumab.

Embodiment 233: The method of Embodiment 231, wherein the antibody or antibody format specific for PD-L1 is selected from atezolizumab, avelumab, durvalumab, and BMS-936559.

Embodiment 234: The method of Embodiment 231, wherein the antibody or antibody format specific for CTLA-4 is selected from ipilimumab (YERVOY), tremelimumab, AGEN1884, and RG2077.

Embodiment 235: The method of Embodiment 229, wherein the tumor is a less-immunogenic tumor and the method elicits a potent immune response in the less-immunogenic tumors.

Embodiment 236: The method of Embodiment 229, wherein the tumor has reduced inflammation (“cold tumor”) and the method converts the tumor to a responsive, inflamed tumor (“hot tumor”).

Embodiment 237: The method of Embodiment 230, wherein the method makes the cancer responsive or more responsive to a checkpoint inhibitor therapy and, optionally one or more chemotherapeutic agents and/or radiotherapy.

Embodiment 238: The method of Embodiment 230, wherein the subject is predicted to be poorly responsive or non-responsive to the checkpoint inhibitor therapy based on expression of one or more of PD-1, PD-L1, or PD-L2, in a subject's biological specimen.

Embodiment 239: The method of Embodiment 229, wherein the subject is predicted to be poorly responsive or non-responsive to an agent that modulates one or more of PD-1, PD-L1, and PD-L2 based on low on expression of PD-1, PD-L1, and PD-L2 in a tumor specimen.

Embodiment 240: The method of Embodiment 229, wherein the subject is predicted to be poorly responsive or non-responsive to an agent that modulates one or more of PD-1, PD-L1, and PD-L2 tumor proportion score (TPS) of less than about 49% for PD-L1 staining.

Embodiment 241: The method of any of Embodiments 218-240, wherein the subject is selected by steps comprising: obtaining from the subject a sample of cells comprising a T cell; and contacting the sample of cells with least one perturbagen selected from Table 4, or a variant thereof, wherein the at least one perturbagen alters a gene signature in the sample of cells.

Embodiment 242: The method of any of Embodiments 218-240, wherein the subject is selected by steps comprising: obtaining from the subject a sample of cells comprising a T cell; and contacting the sample of cells with at least one perturbagen capable of altering a gene signature in a T cell, wherein the at least one perturbagen increases in the sample of cells the expression and/or activity of one or more genes selected from Table 2 designated as an “up” gene in the gene directionality column of Table 2 and/or decreases in the sample of cells the expression and/or activity of one or more genes selected from Table 2 designated as a “down” gene in the gene directionality column of Table 2.

Embodiment 243: The method of Embodiment 242, wherein altering the gene signature comprises an activation of a network module designated in the network module column of Table 2.

Embodiment 244: The method of Embodiment 243, wherein the activation of the network module designated in the network module column of Table 2 comprises modulating expression and/or activity of 2 or more genes within a network module.

Embodiment 245: The method of Embodiment 244, wherein the activation of the network module designated in the network module column of Table 2 comprises modulating expression and/or activity of all of the genes within a network module.

Embodiment 246: The method of Embodiment 245, wherein the activation of the network module designated in the network module column of Table 2 comprises modulating expression and/or activity of 2 or more genes within 2 or more network modules.

Embodiment 247: The method of any of Embodiments 218-246, wherein the population of cells is derived from the subject.

Embodiment 248: The method of any of Embodiments 218-246, wherein the population of cells is not derived from the subject.

Embodiment 249: The method any of Embodiments 247, wherein the population of cells is derived from the subject's bone marrow or the subject's blood.

Embodiment 250: The method of Embodiment 247, wherein the population of cells is fractionated prior to contacting with at least one perturbagen.

Embodiment 251: The method of Embodiment 250, wherein the fractionated cells are enriched for T cells.

Embodiment 252: A method for treating or preventing cancer, comprising administering to a subject in need thereof a population of cells, the population of cells having been contacted with at least one perturbagen selected from Table 4, or a variant thereof, wherein the at least one perturbagen is capable of changing a gene signature in a T cell.

Embodiment 253: The method of Embodiment 252, wherein the population of cells is derived from the subject.

Embodiment 254: The method of Embodiment 252, wherein the population of cells is not derived from the subject.

Embodiment 255: The method of Embodiment 253, wherein the population of cells is derived from the subject's bone marrow or the subject's blood.

Embodiment 256: The method of Embodiment 253, wherein the population of cells is fractionated prior to contacting with at least one perturbagen.

Embodiment 257: The method of Embodiment 256, wherein the fractionated cells are enriched for T cells.

Embodiment 258: The method of Embodiment 252-257, wherein the administering is parenteral, optionally selected from intravenous, intramuscular, intraperitoneal, subcutaneous, intra-articular injection, and infusion.

Embodiment 259: The method of Embodiment 252-257, wherein the administering is intratumoral.

Embodiment 260: The method of Embodiment 252-259, wherein the cancer is a solid tumor.

Embodiment 261: The method of Embodiment 252-259, wherein the cancer is a liquid tumor.

Embodiment 262: The method of any of Embodiments 252-261, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

Embodiment 263: The method of any of Embodiments 252-261, wherein the cancer is a melanoma, optionally selected from metastatic melanoma, cutaneous melanoma, BRAF V600, Merkel cell carcinoma, cutaneous squamous cell carcinoma, a lung cancer, optionally selected from NSCLC, metastatic nonsquamous non-small cell lung cancer, metastatic small cell carcinoma, a head and neck cancer, optionally selected from head and neck squamous cell cancer, a bladder cancer, optionally selected from urothelial carcinoma, locally advanced or metastatic urothelial carcinoma, a breast cancer, optionally selected from triple-negative breast cancer), microsatellite Instability-high cancer, gastric cancer, optionally selected from gastric or gastroesophageal junction adenocarcinoma, metastatic colorectal cancer, cervical cancer, optionally selected from recurrent or metastatic cervical cancer, liver cancer, optionally selected from HCC, hematological disorder, optionally selected from primary mediastinal large B-cell lymphoma, and Hodgkin lymphoma.

Embodiment 264 The method of any of Embodiments 252-263, wherein the cancer is characterized by a tumor expressing one or more of PD-1, PD-L1, PD-L2, CTLA-4, Tim-3, or LAG-3.

Embodiment 265: The method of any of Embodiments 252-263, wherein the cancer is poorly responsive or non-responsive to checkpoint inhibitor therapy or has presented as poorly responsive or non-responsive to checkpoint inhibitor therapy.

Embodiment 266: The method of Embodiment 265, wherein the checkpoint inhibitor therapy is selected from an antibody or antibody format specific for one of PD-1, PD-L1, PD-L2, CTLA-4, Tim-3, or LAG-3.

Embodiment 267: The method of Embodiment 266, wherein the antibody or antibody format specific for PD-1 is selected from nivolumab, pembrolizumab, and pidilizumab.

Embodiment 268: The method of Embodiment 266, wherein the antibody or antibody format specific for PD-L1 is selected from atezolizumab, avelumab, durvalumab, and BMS-936559.

Embodiment 269: The method of Embodiment 266, wherein the antibody or antibody format specific for CTLA-4 is selected from ipilimumab (YERVOY), tremelimumab, AGEN1884, and RG2077.

Embodiment 270: The method of any of Embodiments 252-269, wherein the method elicits a potent immune response in less-immunogenic tumors.

Embodiment 271: The method of any of Embodiments 252-270, wherein the method converts a tumor with reduced inflammation (“cold tumor”) to a responsive, inflamed tumor (“hot tumor”).

Embodiment 272: The method of any of Embodiments 252-271, wherein the method makes the cancer responsive or more responsive to a checkpoint inhibitor therapy and, optionally one or more chemotherapeutic agents and/or radiotherapy.

Embodiment 273: The method of any of Embodiments 252-272, wherein the subject is predicted to be poorly responsive or non-responsive to the checkpoint inhibitor therapy based on expression of one or more of PD-1, PD-L1, or PD-L2, in a subject's biological specimen.

Embodiment 274: The method of Embodiment 273, wherein the subject is predicted to be poorly responsive or non-responsive to an agent that modulates one or more of PD-1, PD-L1, and PD-L2 based on low on expression of PD-1, PD-L1, and PD-L2 in a tumor specimen.

Embodiment 275: The method of Embodiment 273, wherein the subject is predicted to be poorly responsive or non-responsive to an agent that modulates one or more of PD-1, PD-L1, and PD-L2 tumor proportion score (TPS) of less than about 49% for PD-L1 staining.

Embodiment 276: The method of any of Embodiments 252-275, wherein the method improves clinical outcome or response to therapy with an anti-cancer agent as compared to clinical outcome or response to therapy in the absence of the perturbagen.

Embodiment 277: A method for treating or preventing an infection, comprising administering to a subject in need thereof a population of cells, the population of cells having been contacted with at least one perturbagen selected from Table 4, or a variant thereof, wherein the at least one perturbagen is capable of changing a gene signature in a T cell.

Embodiment 278: The method of Embodiment 277, wherein the population of cells is derived from the subject.

Embodiment 279: The method of Embodiment 277, wherein the population of cells is not derived from the subject.

Embodiment 280: The method of Embodiment 278, wherein the population of cells is derived from the subject's bone marrow or the subject's blood.

Embodiment 281: The method of Embodiment 277, wherein the population of cells is fractionated prior to contacting with at least one perturbagen.

Embodiment 282: The method of Embodiment 281, wherein the fractionated cells are enriched for T cells.

Embodiment 283: The method of any of Embodiments 277-282, wherein the infection is selected from bacterial infections, viral infections, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

Embodiment 284: The method of any of Embodiments 277-283, wherein the method improves clinical outcome or response to therapy with an anti-infective agent or vaccine as compared to clinical outcome or response to therapy in the absence of the perturbagen.

Embodiment 285: The method of any of Embodiments 277-284, wherein the administering is parenteral, optionally selected from intravenous, intramuscular, intraperitoneal, subcutaneous, intra-articular injection, and infusion.

Embodiment 286: A method of identifying a candidate perturbation for reducing or reversing the conversion of a T cell into an exhausted T cell, the method comprising: exposing the starting population of T cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of T cells into exhausted T cells following exposure of the population of cells to the perturbation; and identifying the perturbation as a candidate perturbation for reducing or reversing the conversion of a T cell thereof based on the perturbation signature, wherein the perturbation signature is an increase in expression and/or activity in the T cell of one or more genes selected from Table 2 designated as an “up” gene in the gene directionality column of Table 2 and/or decreases in the sample of cells the expression and/or activity of one or more genes selected from Table 2 designated as a “down” gene in the gene directionality column of Table 2.

Embodiment 287: The method of Embodiment 286, wherein altering the perturbation signature comprises an activation of a network module designated in the network module column of Table 2.

Embodiment 288: The method of Embodiment 287, wherein the activation of the network module designated in the network module column of Table 2 comprises modulating expression and/or activity of 2 or more genes within a network module.

Embodiment 289: The method of Embodiment 288, wherein the activation of the network module designated in the network module column of Table 2 comprises modulating expression and/or activity of all of the genes within a network module.

Embodiment 290: The method of Embodiment 289, wherein the activation of the network module designated in the network module column of Table 2 comprises modulating expression and/or activity of 2 or more genes within 2 or more network modules.

Embodiment 291: A method for making a therapeutic agent for a cancer or infection, comprising: (a) identifying a candidate perturbation according to the method of Embodiment 287 and (b) formulating the candidate perturbation as a therapeutic agent for the treatment of the disease or disorder.

Embodiment 292: The method of Embodiment 291, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

Embodiment 293: The method of Embodiment 291, wherein the infection is selected from bacterial infections, viral infections, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

Embodiment 294: An ex-vivo method for directing a change in cell state of a T cell, comprising: contacting a population of cells with at least one perturbagen capable of altering a gene signature in the T cell, wherein altering the gene signature comprises an increase in expression and/or activity in the T cell of one or more genes selected from Table 2 designated as an “up” gene in the gene directionality column of Table 2 and/or a decrease in expression and/or activity in the T cell of one or more genes selected from Table 2 designated as a “down” gene in the gene directionality column of Table 2; wherein the change in cell state is a prevention or reduction of a cellular transition to an exhausted T cell state; and wherein the T cell is selected from an effector T cell, an exhausted T cell, and a naïve T cell.

Embodiment 295: The method of Embodiment 294, wherein the change in cell state is a prevention or reduction of a transition of an effector T cell to an exhausted T cell state.

Embodiment 296: The method of Embodiment 294, wherein the at least one perturbagen is selected from Table 4, or a variant thereof.

Embodiment 297: The method of Embodiment 296, wherein the at least one perturbagen selected from Table 4 comprises at least 2, at least 3, at least 4, or at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, or at least 35, at least 36, at least 37, at least 38, at least 39, at least 40, at least 41, at least 42, or at least 43 perturbagens selected from Table 4, or variants thereof.

Embodiment 298: The method of Embodiment 294, wherein the T cells are CD8+ T cells.

Embodiment 299: The method of Embodiment 294, wherein the one or more genes selected from Table 2 comprises at least one of MYC, TES, CXCR4, IGFBP3, PRSS23, SYPL1, CYB561, CCNH, XBP1, RPS6, ADRB2, GDPD5, SORBS3, ZFP36, FOS, PXN, SLC25A4, DSG2, SATB1, IER3, SSBP2, RPS5, ATP1B1, and GADD45B.

Embodiment 300: The method of Embodiment 294, wherein the one or more genes selected from Table 2 comprises at least one of CDK6, MTHFD2, ID2, SCCPDH, SLC25A46, ETFB, HLA-DRA, CHN1, RAB27A, TBXA2R, NFKB2, ITGAE, SMC4, STMN1, GATA3, ETS1, IQGAP1, CAT, RALA, TSC22D3, CBLB, INPP4B, PLSCR1, NUSAP1, RGS2, EVL, PSMB8, HERPUD1, APBB2, MIF, SQSTM1, PGAM1, TWF2, DRAP1, ETV1, CCNA1, HTRA1, DUSP4, GAPDH, RPA3, ADGRG1, ACOT9, CALM3, SOX4, HMOX1, RHOA, S100A4, ANKRD10, FCHO1, KDM5B, SPTAN1, CTSD, HLA-DMA, FGFR4, SLC1A4, HSPB1, CDKN2A, STAT3, RAC2, TIAM1, RALGDS, and EZH2.

### Methods of Culturing Cells In Vitro to Perform Single-Cell Analyses

In carrying out the techniques described herein for identifying the causes of cell fate, it is useful to generate datasets regarding cellular-component measurements obtained from single-cells. To generate these datasets, a population of cells of interest may be cultured in vitro. Alternately, these datasets may be generated, from single cells that have not been previously cultured; for example, cells used in single cell analyses may be obtained from dissociated primary tissue or from a blood product. This latter method of generating datasets is often desirable if one wants to capture information of the primary cell/organ as close to the in vivo setting as possible. However, for cells undergoing culturing, single-cell measurements of one or more cellular-components of interest may be performed at one or more time periods during the culturing to generate datasets.

In some embodiments, cellular-components of interest include nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc. proteins), lipids, carbohydrates, nucleotides (e.g., adenosine triphosphate (ATP), adenosine diphosphate (ADP) and adenosine monophosphate (AMP) including cyclic nucleotides such as cyclic adenosine monophosphate (cAMP) and cyclic guanosine monophosphate (cGMP), other small molecule cellular-components such as oxidized and reduced forms of nicotinamide adenine dinucleotide (NADP/NADPH), and any combinations thereof. In some embodiments, the cellular-component measurements comprise gene expression measurements, such as RNA levels.

Any one of a number of single-cell cellular-component expression measurement techniques may be used to collect the datasets. Examples include, but are not limited to single-cell ribonucleic acid (RNA) sequencing (scRNA-seq), scTag-seq, single-cell assay for transposase-accessible chromatin using sequencing (scATAC-seq), CyTOF/SCOP, E-MS/Abseq, miRNA-seq, CITE-seq, and so on. The cellular-component expression measurement can be selected based on the desired cellular-component to be measured. For instance, scRNA-seq, scTag-seq, and miRNA-seq measure RNA expression. Specifically, scRNA-seq measures expression of RNA transcripts, scTag-seq allows detection of rare mRNA species, and miRNA-seq measures expression of micro-RNAs. CyTOF/SCOP and E-MS/Abseq measure protein expression in the cell. CITE-seq simultaneously measures both gene expression and protein expression in the cell. And scATAC-seq measures chromatin conformation in the cell. Table A below provides links to example protocols for performing each of the single-cell cellular-component expression measurement techniques described herein.

TABLE A
Example Measurement Protocols

| Technique | Protocol |
|---|---|
| RNA-seq | Olsen and Baryawno “Introduction to Single-Cell RNA Sequencing” Current |
| | Protocols in Molecular Biology. Volume 122, Issue 1, April 2018, e57 |
| Tag-seq | Rozenberg et al., “Digital gene expression analysis with sample multiplexing and |
| | PCR duplicate detection: A straightforward protocol”, BioTechniques, vol. 61, |
| | No. 1, March 2018 |
| ATAC-seq | Buenrostro et al., “ATAC-seq: A Method for Assaying Chromatin Accessibility |
| | Genome-Wide”, Curr Protoc Mol Biol. 2015; 109: 21.29.1-21.29.9 |
| miRNA-seq | Faridani et al., “Single-cell sequencing of the small-RNA transcriptome” Nature |
| | Biotechnology volume 34, pages 1264-1266 (2016) |
| CyTOF/SCoPE-MS/Abseq | Bandura et al., “Mass Cytometry: Technique for Real Time Single Cell |
| | Multitarget Immunoassay Based on Inductively Coupled Plasma Time-of-Flight |
| | Mass Spectrometry”, Anal Chem. 2009 Aug 15; 81(16):6813-22 |
| | Shahi et al., “Abseq: Ultrahigh-throughput single cell protein profiling with droplet |
| | microfluidic barcoding”, Scientific Reports volume 7, Article number: 44447 |
| | (2017) |
| | Budnik et al., “SCoPE-MS: mass spectrometry of single mammalian cells |
| | quantifies proteome heterogeneity during cell differentiation”, Genome Biology |
| | 2018 19: 161 |
| CITE-seq | Stoeckius et al., “Simultaneous epitope and transcriptome measurement in |
| | single cells”, Nature Methods, vol 14, pages 865-868 (2017) |

The cellular-component expression measurement technique used may result in cell death. Alternatively, cellular-components may be measured by extracting out of the live cell, for example by extracting cell cytoplasm without killing the cell. Techniques of this variety allow the same cell to be measured at multiple different points in time.

If the cell population is heterogeneous such that multiple different cell types that originate from a same cell are present in the population, then single-cell cellular-component expression measurements can be performed at a single time point or at relatively few time points as the cells grow in culture. As a result of the heterogeneity of the cell population, the collected datasets will represent cells of various types along a trajectory of transition.

If the cell population is substantially homogeneous such that only a single or relatively few cell types, mostly the “cell of interest, are present in the population, then single-cell cellular-component expression measurements can be performed multiple times over a period of time as the cells transition.

A separate single-cell cellular-component expression dataset is generated for each cell, and where applicable at each of the time periods. The collection of single-cell cellular-component expression measurements from a population of cells at multiple different points in time can collectively be interpreted as a “pseudo-time” representation of cell expression over time for the cell types originating from the same cell. The term pseudo-time is used in two respects, first, in that cell state transition is not necessarily the same from cell to cell, and thus the population of cell provides a distribution of what transition processes a cell of that type is likely to go through over time, and second, that the cellular-component expression measurements of those multiple cell's expressions at multiple time points simulates the possible transition behavior over time, even if cellular-component expression measurements of distinct cells give rise to the datasets. As a deliberately simple example, even if cell X gave a dataset for time point A and cell Y gave a dataset for time point B, together these two datasets represent the pseudo-time of transition between time point A and time point B.

For convenience of description, two such datasets captured for a “same” cell at two different time periods (assuming a technique is used that does not kill the cell) are herein referred to as different “cells” (and corresponding different datasets) because in practice such cells will often be slightly or significantly transitioned from each other, in some cases having an entirely distinct cell type as determined from the relative quantities of various cellular-components. Viewed from this context, these two measurements of a single-cell at different time points can be interpreted as different cells for the purpose of analysis because the cell itself has changed.

Note that the separation of datasets by cell/time period described herein is for clarity of description, in practice, these datasets may be stored in computer memory and logically operated on as one or more aggregate dataset/s (e.g., by cell for all time periods, for all cells and time periods at once).

In some instances, it is useful to collect datasets where a cell of interest has been perturbed from its base line state. There are a number of possible reasons to do this, for example, to knock out one or more cellular-components, to evaluate the difference between healthy and diseased cell states. In these instances, a process may also include steps for introducing the desired modifications to the cells. For example, one or more perturbations may be introduced to the cells, tailored viruses designed to knock out one or more cellular-components may be introduced, clustered regularly interspaced short palindromic repeats (CRISPR) may be used to edit cellular-components, and so on. Examples of techniques that could be used include, but are not limited to, RNA interference (RNAi), Transcription activator-like effector nuclease (TALEN) or Zinc Finger Nuclease (ZFN).

Depending upon how the perturbation is applied, not all cells will be perturbed in the same way. For example, if a virus is introduced to knockout a particular gene, that virus may not affect all cells in the population. More generally, this property can be used advantageously to evaluate the effect of many different perturbations with respect to a single population. For example, a large number of tailored viruses may be introduced, each of which performs a different perturbation such as causing a different gene to be knocked out. The viruses will variously infect some subset of the various cells, knocking out the gene of interest. Single-cell sequencing or another technique can then be used to identify which viruses affected which cells. The resulting differing single-cell sequencing datasets can then be evaluated to identify the effect of gene knockout on gene expression in accordance with the methods described elsewhere in this description.

Other types of multi-perturbation cell modifications can be performed similarly, such as the introduction of multiple different perturbations, barcoding CRISPR, etc. Further, more than one type perturbation may be introduced into a population of cells to be analyzed. For example, cells may be affected differently (e.g., different viruses introduced), and different perturbations may be introduced into different sub-populations of cells.

Additionally, different subsets of the population of cells may be perturbed in different ways beyond simply mixing many perturbations and post-hoc evaluating which cells were affected by which perturbations. For example, if the population of cells is physically divided into different wells of a multi-well plate, then different perturbations may be applied to each well. Other ways of accomplishing different perturbations for different cells are also possible.

Below, methods are exemplified using single-cell gene expression measurements. It is to be understood that this is by way of illustration and not limitation, as the present disclosure encompasses analogous methods using measurements of other cellular-components obtained from single-cells. It is to be further understood that the present disclosure encompasses methods using measurements obtained directly from experimental work carried out by an individual or organization practicing the methods described in this disclosure, as well as methods using measurements obtained indirectly, e.g., from reports of results of experimental work carried out by others and made available through any means or mechanism, including data reported in third-party publications, databases, assays carried out by contractors, or other sources of suitable input data useful for practicing the disclosed methods.

As discussed herein, gene expression in a cell can be measured by sequencing the cell and then counting the quantity of each gene transcript identified during the sequencing. In some embodiments, the gene transcripts sequenced and quantified may comprise RNA, for example mRNA. In alternative embodiments, the gene transcripts sequenced and quantified may comprise a downstream product of mRNA, for example a protein such as a transcription factor. In general, as used herein, the term “gene transcript” may be used to denote any downstream product of gene transcription or translation, including post-translational modification, and “gene expression” may be used to refer generally to any measure of gene transcripts.

Although the remainder of this description focuses on the analysis of gene transcripts and gene expression, all of the techniques described herein are equally applicable to any technique that obtains data on a single-cell basis regarding those cells. Examples include single-cell proteomics (protein expression), chromatin conformation (chromatin status), methylation, or other quantifiable epigenetic effects.

The following description provides an example general description for culturing a population of cells in vitro in order to carry out single-cell cellular-component expression measurement multiple time periods. Methods for culturing cells in vitro are known in the art. Those of skill in the art will also appreciate how this process could be modified for longer/shorter periods, for additional/fewer single-cell measurement steps, and so on.

In one embodiment, the process for culturing cells in a first cell state into cells in a second cell state includes one or more of the following steps:
Day 0: Thaw cells in the first cell state into a plate in a media suitable for growth of the cells.
Day 1: Seed cells in the first cell state into a multi-well plate. If applicable, perform additional steps to affect gene expression by cells. For example, simultaneously infect with one or more viruses to activate or knock out genes of interest.
   Perform gene expression measurement iteration t₁ for cells in the wells.
Day 1+I: Change media as needed if any additional processes are performed.
   If applicable, perform gene expression measurement iteration tₗ for cells in the wells.
Day 1+m: Change media to media appropriate to support growth of cells in the second cell state.
   If applicable, perform gene expression measurement iteration tₘ for cells in the wells.
Days 1+n, o, p, etc.: Media change as needed to support further cell state transition from the first cell state to the second cell state. If applicable, perform additional steps to affect further transition from the first cell state to the second cell state. For example, add perturbations of interest to push cells towards the second cell state.
   If applicable, perform gene expression measurement iterations tₙ, tₒ, tₚ, etc., for cells in the wells.
Day q: Perform gene expression measurement iteration t_{q} for cells in the wells and in the second state.
Collect cells into a tube and stain in suspension with antibodies matched to genes/proteins of interest, thereby sorting/identifying cells without having to lyse/destroy them. This step also can identify surface proteins that might not be seen with as much resolution in the setting of the cytoplasm. Image with a cell imaging system such as the BD Celestra flow cytometer or similar instrument by acquiring the cells from each well or tube. Quantify of number of cells per well that are in the first cell state and the number of cells per well that are in the second cell state. These steps can be used with unfixed cells.

Any aspect or embodiment disclosed herein can be combined with any other aspect or embodiment as disclosed herein.

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the disclosure. Certain terms are discussed herein to provide additional guidance to the practitioner in describing the compositions, the devices, the methods and the like of aspects of the disclosure and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

The term “perturbation” in reference to a cell (e.g., a perturbation of a cell or a cellular perturbation) refers to any treatment of the cell with one or more active agents capable of causing a change in a cell state. These active agents can be referred to as “perturbagens.” In embodiments, the perturbagen can comprise, e.g., a small molecule, a biologic, a protein, a protein combined with a small molecule, an antibody-drug conjugate (ADC), a nucleic acid, such as an siRNA or interfering RNA, a cDNA over-expressing wild-type and/or mutant shRNA, a cDNA over-expressing wild-type and/or mutant guide RNA (e.g., Cas9 system, Cas9-gRNA complex, or other gene editing system), or any combination of any of the foregoing. As used herein, a perturbagen classified as a “compound” may be a small molecule or a biologic. Also, a perturbagen classified as “overexpression of gene” may be cDNA over-expressing a wild-type gene or an mRNA encoding a wild-type gene. In embodiments, an mRNA may comprise a modified nucleotide that promotes stability of the mRNA and/or reduces toxicity to a subject. Examples of modified nucleotides useful in the present disclosure include pseudouridine and 5-methylcytidine. Where a perturbagen is (or includes) a nucleic acid or protein described by reference to a particular sequence, it should be understood that variants with similar function and nucleic acid or amino acid identity are encompassed as well, e.g., variants with about: 1%, 2%, 3%, 4%, 5%, 6%, 7%, 8%, 9%, 10%, 11%, 12%, 13%, 14%, 15%, or more, variation, i.e., having about: 99%, 98%, 97%, 96%, 95%, 94%, 93%, 92%, 91%, 90%, 89%, 88%, 87%, 86%, or 85% identity to the reference sequence; e.g., in some embodiments, having, for example, at least: 1, 2, 3, 4, 5, 6, 7, 8, 9, 10, 11, 12, 13, 14, 15, 20, 25, 30, 35, 40, 45, 50, or more, substitutions.

As used herein, the terms “cell fate” and “cell state” are interchangeable and synonymous.

The term “subject,” refers to an individual organism such as a human or an animal. In embodiments, the subject is a mammal (e.g., a human, a non-human primate, or a non-human mammal), a vertebrate, a laboratory animal, a domesticated animal, an agricultural animal, or a companion animal. In embodiments, the subject is a human (e.g., a human patient). In embodiments, the subject is a rodent, a mouse, a rat, a hamster, a rabbit, a dog, a cat, a cow, a goat, a sheep, or a pig.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”. Likewise, the term “and/or” covers both “or” and “and”.

Unless specifically stated or obvious from context, as used herein, the term “about” is understood as within a range of normal tolerance in the art, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The disclosure will be further described in the following examples, which do not limit the scope of the disclosure described in the claims.

### EXAMPLES

### Example 1: Screen Small Molecules Predicted to Prevent T Cell Exhaustion in a Syngeneic Mouse Model of Colon Adenocarcinoma

Using published scRNAseq datasets derived from a well validated mouse model of chronic T cell exhaustion (lymphocytic choriomeningitis virus, LCMV, clone 13), we generated gene signatures for T cell exhaustion. Using these signatures, we made predictions of small molecules likely to prevent the transition of T cells from an effector to an exhausted state. We hypothesized that a block of T cell exhaustion in a syngeneic mouse model would induce tumor growth inhibition. To test this hypothesis, we performed an efficacy study in MC38 syngeneic mice and at the end of the study we analyzed tumors to characterize infiltrating T lymphocytes (TILs) using flow cytometry and scRNAseq.

Female C57/Bl6 mice (Taconic, Albany, New York) were inoculated at 7 weeks of age with the colon adenocarcinoma cell line, MC38 (1×10⁵ cells in 100 μl PBS: Matrigel mix, 50:50). Once the mean tumor volume had reached 100 mm², mice were divided into groups of 5 for each treatment arm (Table 5). Perturbagens referenced in this Example refer to perturbagens of Table 3.

TABLE 5
Test Compounds, Dosing and Route of Administration

| | | | | | Route Admin./dosing |
|---|---|---|---|---|---|
| Article No. | Compound | Dose | | Formulation | schedule |
| 1 | Vehicle (PBS) | N/A | | — | IP, QDx10 |
| 2 | Anti-PD-1 | 200 | ug | 200 ug | IP, BIWx3 |
| 3 | Perturbagen 4 | 30 | mg/kg | DRD | IP, QDx10 |
| 4 | Perturbagen 15 | 10 | mg/kg | DRD | IP, QDx10 |
| 5 | Perturbagen 3 | 3 | mg/kg | DRD | IP, QDx10 |
| 6 | Perturbagen 17 | 30 | mg/kg | DRD | PO, QDx10 |
| 7 | Perturbagen 18 | 30 | mg/kg | DRD | IP, QDx10 |
| 8 | Perturbagen 13 | 30 | mg/kg | PBS | PO, QDx10 |
| 9 | Perturbagen 10 | 3 | mg/kg | DRD | PO, QDx10 |
| 10 | Perturbagen 19 | 30 | mg/kg | DRD | IP, QDx10 |
| 11 | Perturbagen 9 | 3 | mg/kg | DRD | PO, QDx10 |
| 12 | Perturbagen 17 | 3 | mg/kg | DRD | IP, QDx10 |
| 13 | Perturbagen 2 | 5 | mg/kg | DRD | IP, QDx10 |
| 14 | Perturbagen 12 | 3 | mg/kg | DRD | PO, QDx10 |
| 15 | Perturbagen 1 | 10 | mg/kg | DRD | IP, QDx10 |
| 16 | Perturbagen 16 | 30 | mg/kg | DRD | PO, QDx10 |
| 17 | Perturbagen 11 | 10 | mg/kg | DRD | IP, QDx10 |
| 18 | Perturbagen 8 | 10 | mg/kg | DRD | IP, QDx10 |
| 19 | Perturbagen 5 | 3 | mg/kg | DRD | IP, QDx10 |
| 20 | Perturbagen 14 | 10 | mg/kg | DRD | IP, QDx10 |
| 21 | Perturbagen 6 | 30 | mg/kg | DRD | IP, QDx10 |
| 22 | Perturbagen 20 | 3 | mg/kg | DRD | IP, QDx10 |

Compounds were dissolved in DMSO as 10× stocks and aliquoted for each day of dosing. For dosing, an aliquot of stock compound was diluted into DRD (10% DMSO, 90% D5W: cremaphor: 80-20 mixture; D5W, 5% dextrose in water) to give the appropriate final concentration for dosing. All body weights were recorded prior to each dose and compounds were dosed daily for 10 days either by oral gavage or intraperitoneal injection. Tumor size was determined using digital calipers every 2 days and all data were graphed as the experiment progressed (FIG. 1B).

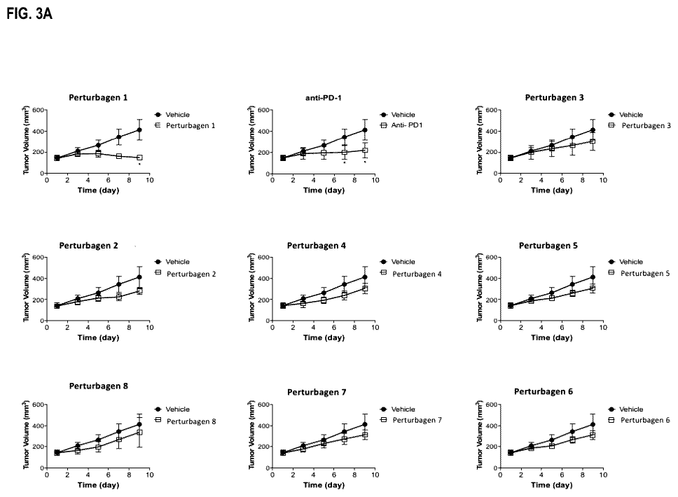

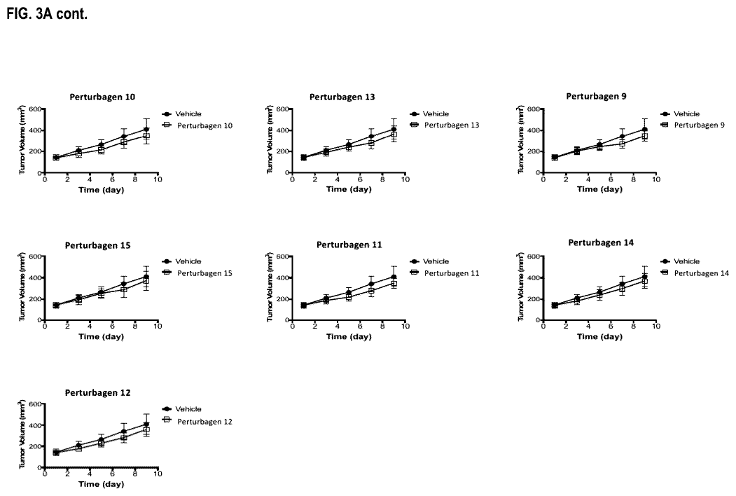

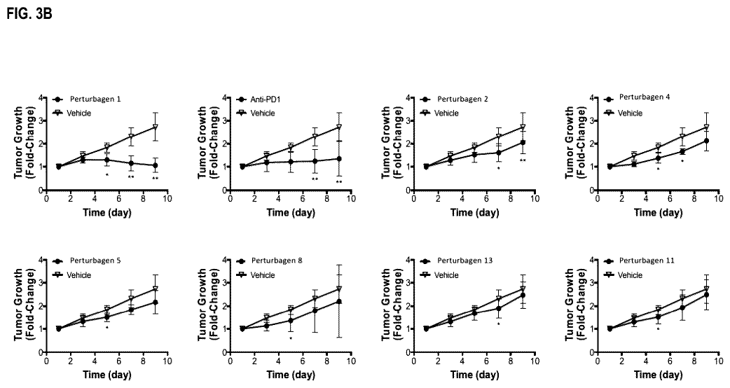

After 24 hours of the first dose, we noted that 2 test compounds were lethal at the doses chosen: Perturbagen 19 at 30 mg/kg and Perturbagen 20 at 3 mg/kg. However, no adverse effects or significant change in body weight were observed with any other test compound for the duration of the study. We noted that our positive control, pembrolizumab, produced significant tumor growth inhibition (TGI) by day 9. One of our test compounds, Perturbagen 1, produced greater TGI than pembrolizumab. Although not significant, many of the remaining test compounds (14 of 17; 82%) showed a trend towards TGI (FIGS. 3A & 3B).

The day after the final dose, mice were euthanized, blood was collected by cardiac puncture and the tumors were removed. For all mice, a section of tumor was flash frozen in liquid nitrogen and stored at −80 degrees C. for future analysis; the remainder of the tumor was placed in cold RPMI (with glucose) and processed to generate a single cell suspension for flow cytometry. For a pre-selected subgroup of mice, a sample of the single cell suspension was collected for scRNAseq (3 samples from 5 treatment groups provided 15 samples for 2 runs on a 10× Chromium box). Each tumor was minced with a clean razor blade to the consistency of chopped garlic. The tumor was placed in a digestive enzyme mix (mouse tumor dissociation kit, Miltenyi) and incubated on a dissociator at 37 degrees C. for 45 min (OctoMax). Following dissociation, the slurry was filtered through a 70 μm nylon filter, centrifuged, and the pellet resuspended in Ammonium-Chloride-Potassium (ACK) lysing buffer for 3 minutes to lyse red blood cells. The ACK buffer was neutralized with 10-fold excess PBS and then the cell suspension was centrifuged to concentrate the cells. Cell suspensions from all tumors were stored on ice until analyzed by flow cytometry.

For flow cytometry, samples of each cell suspension were incubated at room temperature for one hour with an antibody panel optimized for detection of effector and exhausted T cells (Tables 6 and 7). Prior to intracellular staining, samples were permeabilized and fixed using the eBioscience™ Foxp3 Transcription Factor Staining Buffer Set (ThermoFisher) according to the manufacturer's recommendations. Samples were then incubated for one hour with the intracellular staining mix, then centrifuged and resuspended for flow cytometric analysis. Following compensation measurements using commercial beads, fluorescence minus one (fmo) measurements were performed followed by measurements of all the samples. For each sample, at least 100,000 events were recorded. All analysis for flow cytometry data was performed using FlowJo™ (BD).

TABLE 6
Panel of Antibodies Used in Flow Cytometry of
Tumor Samples to Identify T-cell Phenotypes

| | | | Attune | | | |
|---|---|---|---|---|---|---|
| No. | Marker | Conjugate | Filter | Vendor | Catalog # | Clone |
| 1 | Viability | APC-eFluor 780 | RL3 | ThermoFisher | 65-0865-14 | N/A |
| 2 | CD45 | FITC | BL1 | ThermoFisher | 11-0451-82 | 30-F11 |
| 3 | CD3 | PE-Cy7 | YL4 | BioLegend | 100220 | 17A2 |
| 4 | CTLA-4 | PerCP-Cy5.5 | BL3 | BioLegend | 106316 | UC10-4B9 |
| 5 | CD8 | BV 510 | VL2 | Biolegend | 100752 | 53-6.7 |
| 6 | TCF7 | PE | YL1 | BD Biosciences | 564217 | S33-966 |
| 7 | PD-1 | PE-Dazzle 594 | YL2 | BioLegend | 135228 | 29F.1A12 |
| 8 | TIM-3 | BV 421 | VL1 | BD Biosciences | 747626 | 5D12 |
| 9 | TIGIT | BV 711 | VL4 | BD Biosciences | 744214 | 1G9 |
| 10 | LAG-3 | APC | RL1 | Miltenyi | 130-102-527 | C9B7W |
| 11 | CD25 | Alexa Fluor 700 | RL2 | BioLegend | 102024 | PC61 |

TABLE 7
Cell Types and Staining

| Marker | Cell type/physiology |
|---|---|
| CD45 | Leucocyte marker |
| CD3 | T-cell marker |
| CTLA-4 | Inhibitory receptor |
| CD8 | Co-receptor of the T-cell receptor |
| TCF7 | Transcription factor that drives |
| | inhibitory receptor expression |
| PD-1 | Inhibitory receptor |
| TIM-3 | Inhibitory receptor |
| TIGIT | Inhibitory receptor |
| LAG-3 | Inhibitory receptor |
| CD25 | IL-2 receptor |

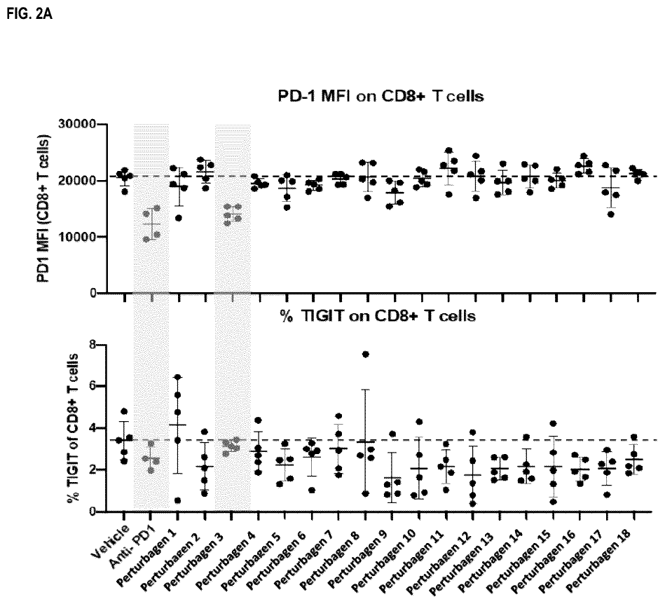

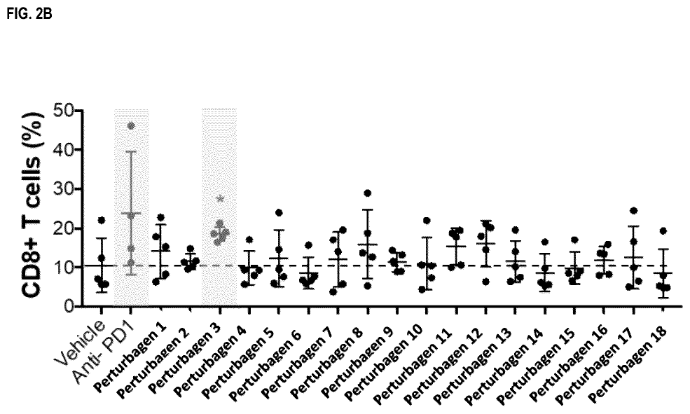

The positive control, pembrolizumab, reduced the expression of PD-1 and TIGIT in CD8+ T cells derived from tumors (FIG. 2A) and increased the number of CD8+ T cells in the tumor (FIG. 2B). These data indicate that a block of checkpoint inhibition and T cell exhaustion are the cause of tumor growth inhibition in this MC38 syngeneic mouse model. Interestingly, Perturbagen 3 produced similar-if not cleaner-reduction in PD-1 and TIGIT expression and an increase in tumor CD8+ T cells (FIGS. 2A & B).

As detailed above, 15 samples of tumor single cell suspension were obtained for single cell RNA sequencing (Table 8). The samples were divided for 2 separate runs on a 10× chromium box to generate shortread sequencer compatible libraries. Samples were subsequently sequenced separately using a nextSeq (Illumina) with version 2.5 chemistry.

TABLE 8
Tumor Cell Suspension Samples Processed for scRNAseg

| Sample No. | Mouse | Treatment |
|---|---|---|
| 1 | XM6169 | PBS |
| 2 | XM6203 | PBS |
| 3 | XM6236 | PBS |
| 4 | XM6142 | Pembrolizumab |
| 5 | XM6233 | Pembrolizumab |
| 6 | XM6114 | Perturbagen 4 |
| 7 | XM6190 | Perturbagen 4 |
| 8 | XM6253 | Perturbagen 4 |
| 9 | XM6132 | Perturbagen 1 |
| 10 | XM6152 | Perturbagen 1 |
| 11 | XM6214 | Perturbagen 1 |
| 12 | XM6130 | Perturbagen 8 |
| 13 | XM6163 | Perturbagen 8 |
| 14 | XM6194 | Perturbagen 8 |

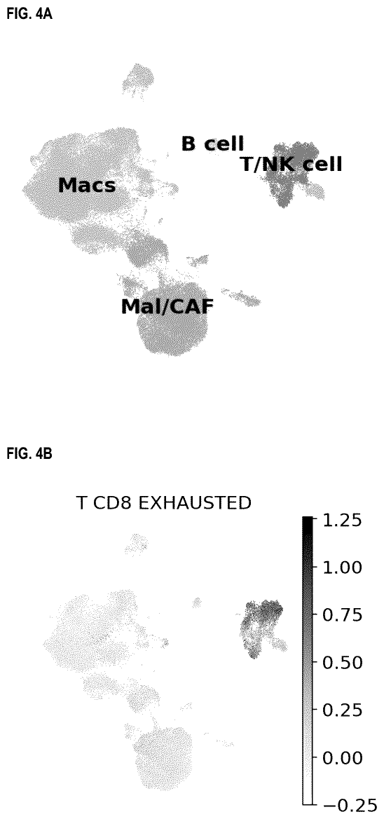

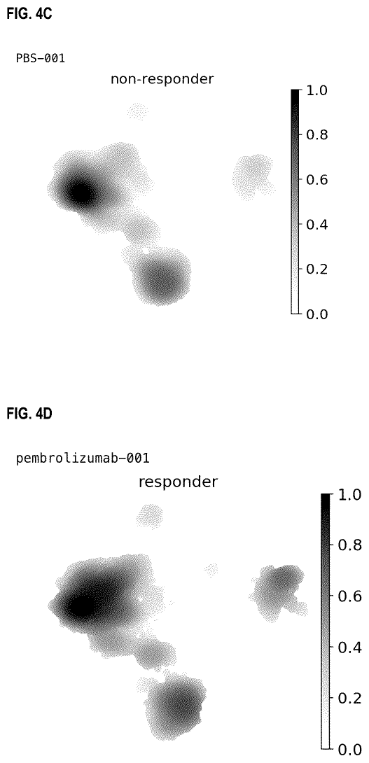

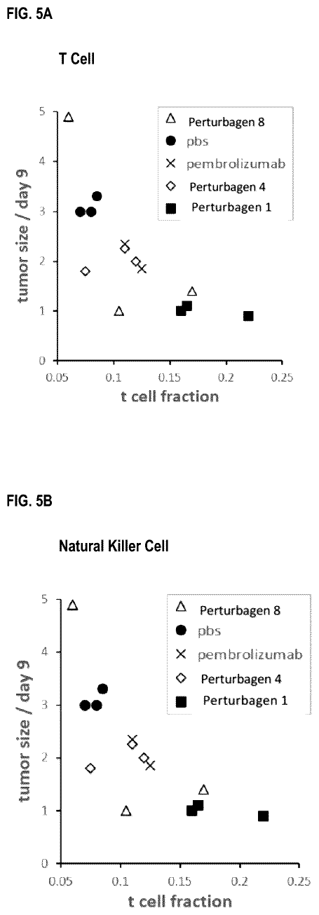

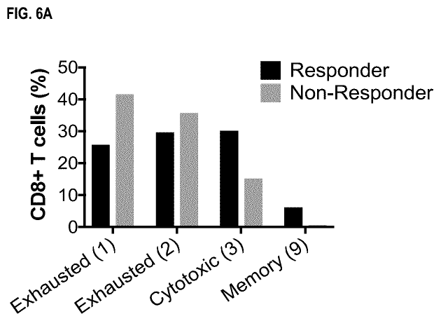

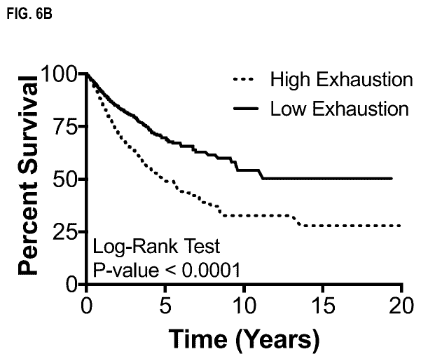

Analysis of all pooled single cell data produced a unique manifold with multiple identifiable cell types (FIGS. 4A-4D). Further, we noted that across all treatment arms there was an increased number of T cells and natural killer cells associated with a decrease in tumor size (FIG. 5A-5B). FIG. 6A shows memory and cytotoxic CD8+ T cells are elevated in responders to checkpoint inhibition, compared to non-responders who have elevated exhausted CD8+ T cells. FIG. 6B shows Kaplan-Meier Curve in adenocarcinoma, stratified by CD8+ T cell exhaustion status (22 gene signature).

### Example 2: Identification of a Small Molecule that Prevent T Cell Exhaustion Using Machine Learning Algorithms Paired with High-Resolution Single Cell RNAseq and Validation of its Mechanism of Action Via Mild TCR Inhibition

T cell responses are tightly regulated and require a constant balance of signals during the different stages of their activation, expansion, and differentiation. As a result of chronic antigen exposure, T cells become exhausted in solid tumors, preventing them from controlling tumor growth. A transcriptional signature associated with T cell exhaustion was identified in patients with melanoma and the machine learning platform predicted molecules that would prevent T cell exhaustion and improve T cell function. Among the predictions, an orally available small molecule, Compound A (C₂₄H₂₀Cl₂FN₅O₂; MW: 500.35 g/mol), was highly predicted.

Compound A was tested in an in vitro T cell exhaustion assay and shown to prevent loss of proliferation and expression of multiple immune checkpoint receptors under exhaustion inducing conditions. Transcriptionally, Compound A-treated cells looked indistinguishable from conventionally expanded, non-exhausted T cells. However, when assessed in a classical T cell activation assay, Compound A demonstrated dose dependent activity. At low dose, Compound A was immuno-stimulatory, allowing cells to divide further by preventing activation induced cell death. At higher doses, Compound A demonstrated immuno-suppressive activity preventing early CD69 upregulation and T cell proliferation. While not wishing to be bound by any particular theory, all together these observations suggest that Compound A prevented exhaustion with a mechanism of action involving TCR signaling inhibition. While cessation of TCR signaling or rest has been recently associated with improved chimeric antigen receptor T-cell (CAR-T) efficacy by preventing or reversing exhaustion during the in vitro manufacturing phase, it is unclear if that mechanism would translate in vivo.

Compound A was evaluated in the CT26 and MC38 syngeneic mouse models alongside anti-PD1. At low dose Compound A closely recapitulated anti-PD1 mediated cell behavior changes by scRNA-seq and flow cytometry in CT26 mice. At high dose, Compound A led to the accumulation of naive cells in the tumor microenvironment (TME) confirming the proposed mechanism of action. Low dose treatment was ineffective in MC38 mouse model but a pulsed treatment at high dose also recapitulated anti-PD1 activity in most animals. Importantly, a new T cell population responding to anti-PD1 that was particularly increased in the MC38 mouse model was identified; Compound A treatment also impacted this population.

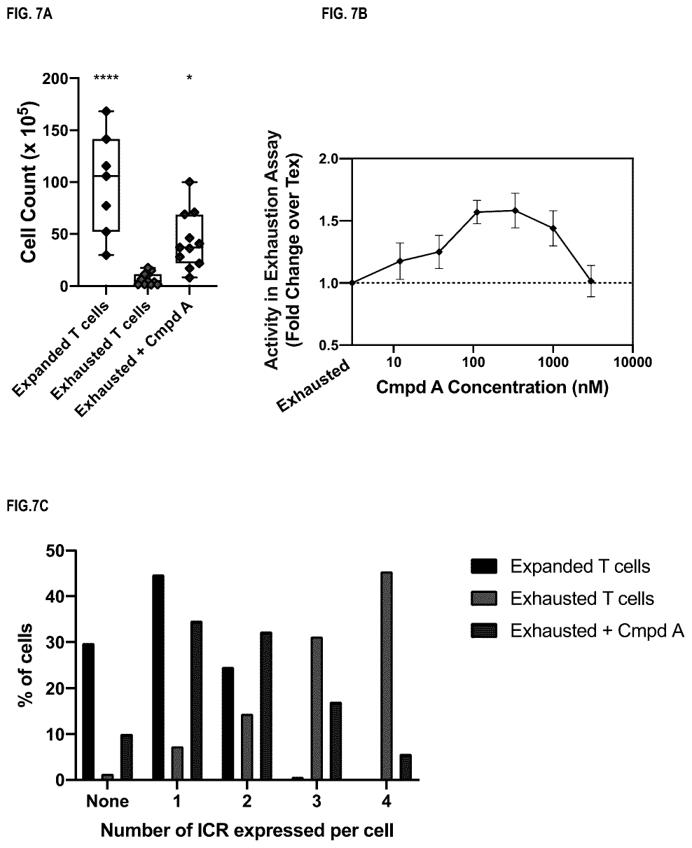

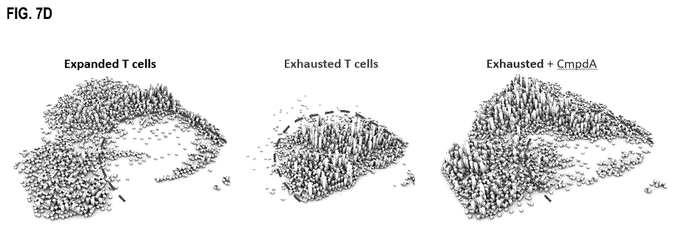

Compound A was found to prevent T cell exhaustion in vitro. Compound A was assessed in a CD8 T cell exhaustion assay. Human CD8 T cells were expanded in vitro or exhausted through repeated stimulations (FIGS. 7A-7D). Cell number was assessed (FIGS. 7A-7B). Compound A at 100 nM restored proliferation under exhaustion condition (data from n=7-11 donors per group, One-way ANOVA, followed by posthoc Dunnet's Vs Exhausted * p<0.05, **** p<0.0001) (FIG. 7A). Cell number was assessed by flow cytometry (FIG. 7B). Compound A restored proliferation under exhaustion condition in a dose dependent manner (pooled data from n=5 donors+/−SEM). Compound A prevents expression of multiple immune checkpoint receptors (ICR). Expression of immune checkpoint receptors PD1, TIM3, LAG3 and TIGIT was assessed by flow cytometry (FIG. 7C). Transcriptionally, Compound A treated exhausted cells are indistinguishable from non-exhausted cells. 10× single cell RNA sequencing data was used to cluster cells by expression profile creating a distribution map of the cell population (FIG. 7D).

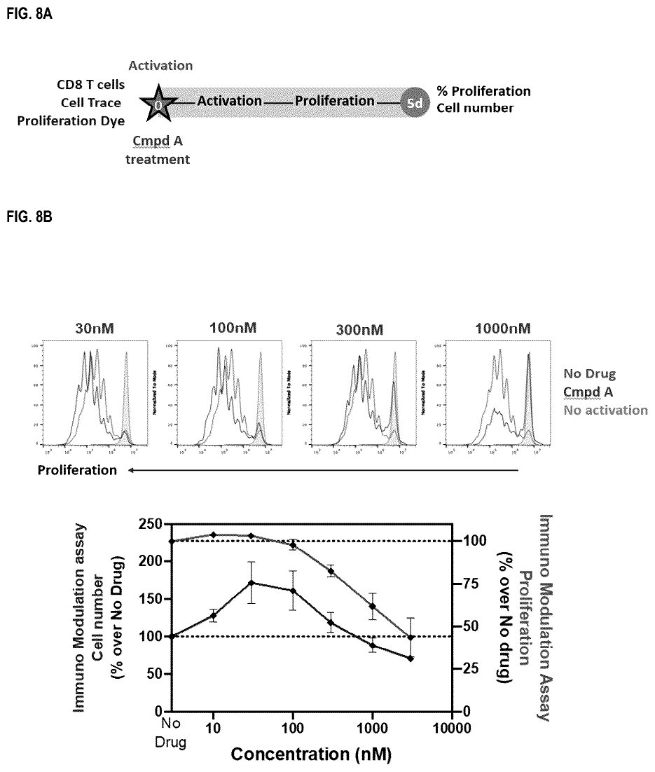

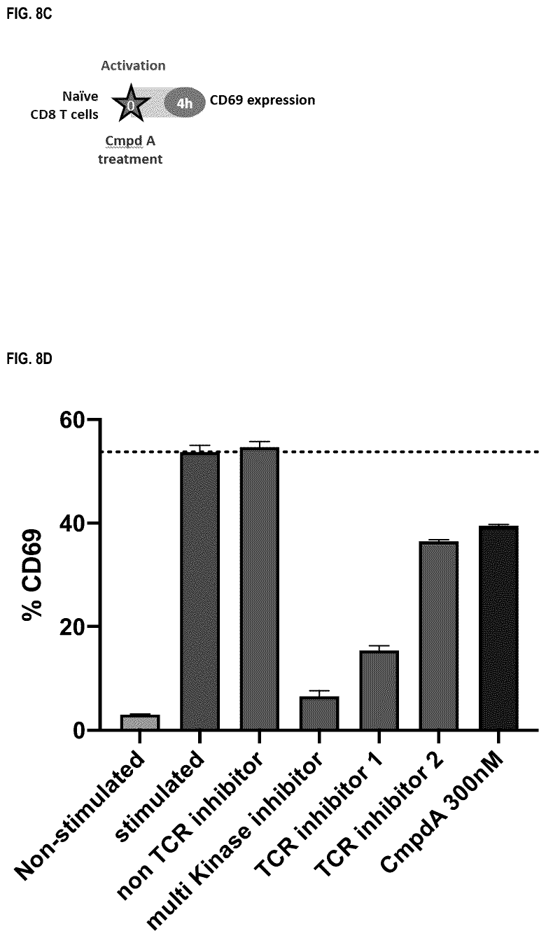

Compound A was found to be immuno-modulatory via mild TCR inhibition (FIGS. 8A-8D). FIG. 8A shows a schematic of an immuno-modulation assay. Compound A demonstrated dose-dependent activity in an immuno-modulatory assay (FIG. 8B). At low dose (<100 nM), Compound A was immuno-stimulatory, allowing cells to divide further and accumulate by preventing activation induced cell death. At higher doses (>100 nM), Compound A was immuno-suppressive and prevented T cell proliferation (pooled data from n=3 donors+/−SEM). FIG. 8C shows a schematic of a TCR inhibition assay. Compound A at 300 nM partially inhibited early CD69 expression following TCR activation assessed by flow cytometry (FIG. 8D).

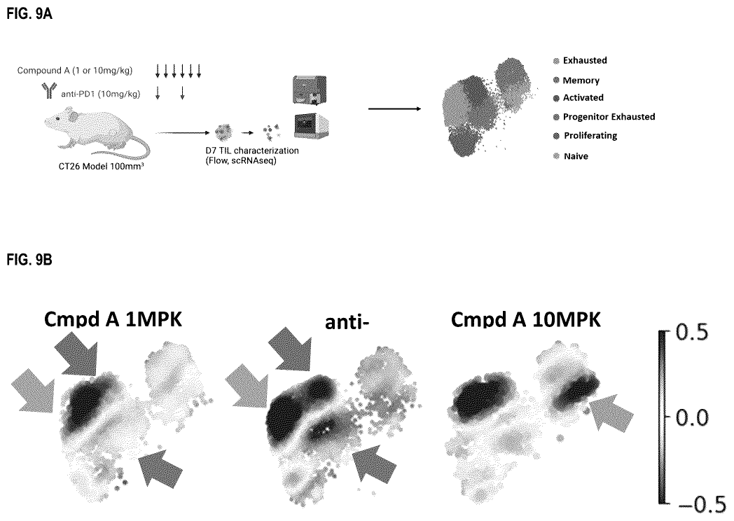

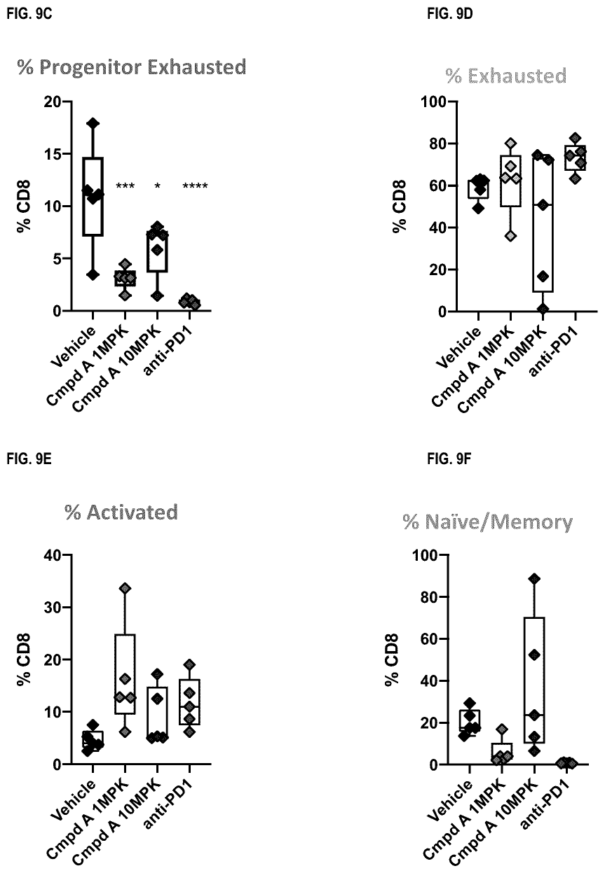

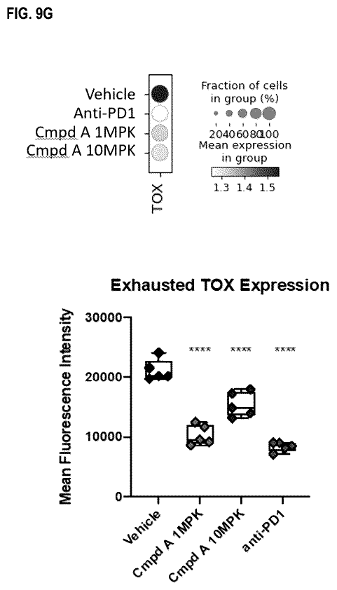

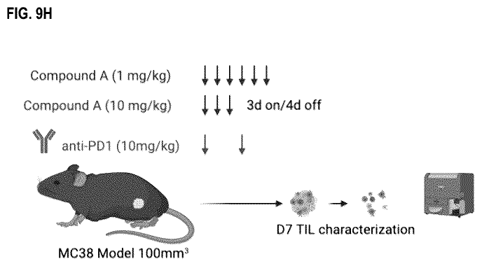

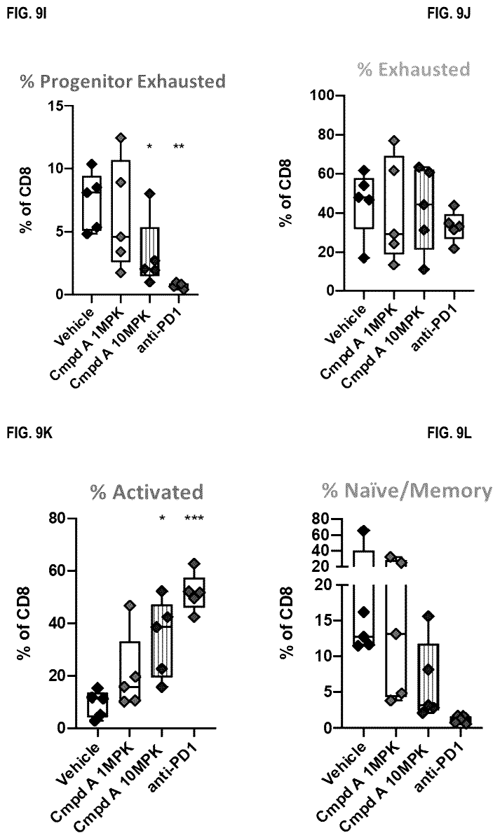

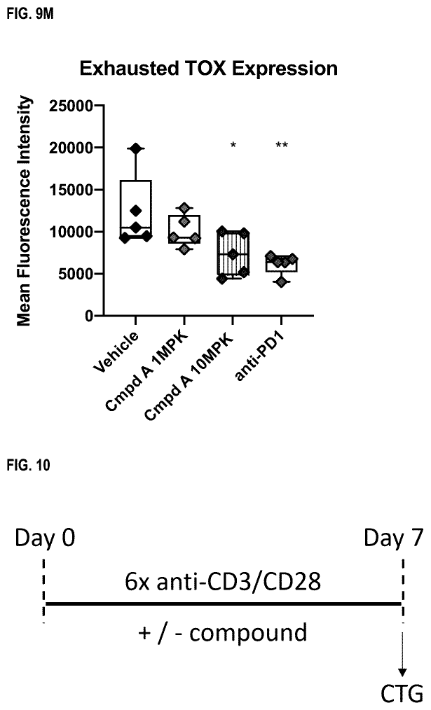

Compound A reduced T cell exhaustion in vivo but with limited window of activity (FIGS. 9A-9M). FIG. 9A shows a non-limiting example of workflow of Compound A evaluation in CT26 model. Compound A treatment at low dose recapitulates anti-PD1 mediated population changes while high dose treatment induces accumulation of naïve cells. FIGS. 9B-9F show analysis of CD8 T cells population changes following 7 days treatment with Compound A by scRNAseq and flow cytometry (FIG. 9B). Leiden Clustering of CD8 T cell populations normalized to vehicle and proportion of CD8 T cell populations were analyzed by flow cytometry (progenitor exhausted TCF7+TOX+ (FIG. 9C), exhausted TCF7−TOX+ (FIG. 9D), activated TCF7−TOX−(FIG. 9E), naïve/memory TCF7+TOX−(FIG. 9F). Compound A treatment reduced TOX expression in exhausted cells. FIG. 9G shows quantification of TOX expression on exhausted cells by scRNAseq and flow cytometry. FIG. 9H shows a non-limiting example of workflow of Compound A evaluation in MC38 model. Compound A pulsed treatment at high dose recapitulated anti-PD1 activity and reduced TOX expression on exhausted cells in most animals. FIGS. 9I-9M show the proportion of CD8 T cells populations and TOX expression on exhausted T cells analyzed by flow cytometry (progenitor exhausted TCF7+TOX+ (FIG. 9I), exhausted TCF7−TOX+ (FIG. 9J), activated TCF7−TOX−(FIG. 9K), naïve/memory TCF7+TOX−(FIG. 9L). One way ANOVA was followed by posthoc Dunnett's Vs Vehicle *p<0.05, ** p<0.01, *** p<0.001, *** p<0.0001, **** p<0.00001 (N=5 mice per group).

In summary, small molecules that prevent T cell exhaustion were identified by the machine learning platform using scRNA-seq data. Compound A was identified as a molecule sufficient to prevent exhaustion in both in vitro and in vivo models of T cell exhaustion, and Compound A efficacy is likely acting through mild TCR inhibition.

All together, these data confirm that mild TCR inhibition either suboptimal or fractionated can prevent exhaustion in vivo. These results demonstrate that this machine learning platform was able to predict molecules that would prevent T cell exhaustion in vivo. In a non-limiting embodiment, this approach can also be used to identify novel non-TCR mediated modulators of T cell exhaustion fit for use in the clinic.

### Example 3: Screening of Small Molecules Predicted to Prevent T Cell Exhaustion in Purified Human CD8+ T Cells

Using published scRNAseq datasets derived from studies characterizing the T cell populations in human melanoma subjects, gene signatures for T cell exhaustion were generated. With these signatures, predictions of small molecules likely to prevent the transition of T cells from an effector to an exhausted state were made. It was determined if these small molecules could prevent T cell exhaustion in purified human CD8+ T cells.

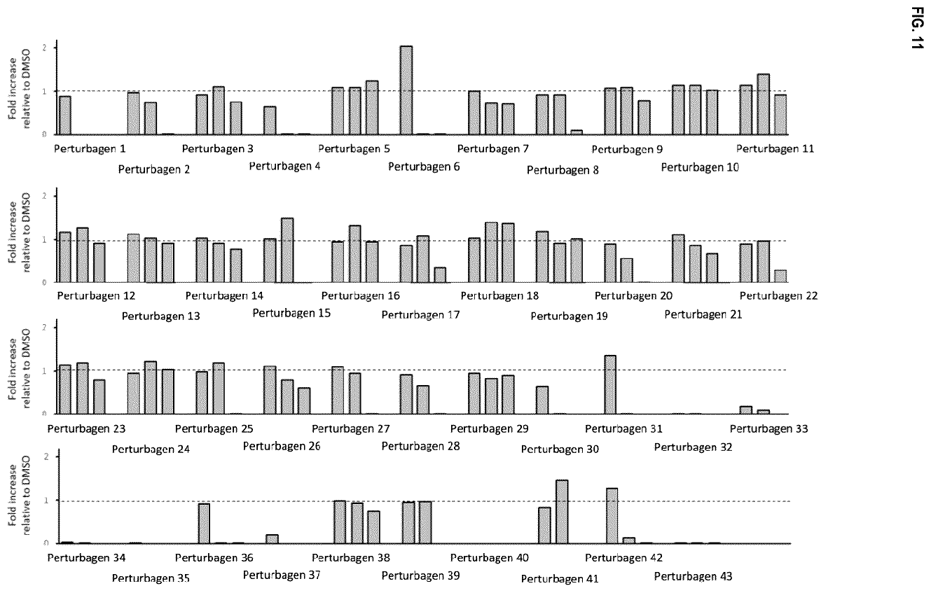

Purified CD8+ T cells (STEMCELL TECHNOLOGIES) were thawed into RPMI1640 media supplemented with FBS (10%), HEPES (10 mM), penicillin (50 units/ml)/streptomycin (50 μg/ml) and L-glutamate (2 mM). Cells were spun down (300 g, 5 minutes) and resuspended into culture media. Cells were seeded into 96-well flat bottom plates (40,000 cells in 90 μl media) and 15 μl anti-CD3/CD28 (Immunoclut, STEM CELL TECHNOLOGIES) was added (this is equivalent to a 6× stimulus). To appropriate wells 10 μl of 10× compound stock solutions was added to give final concentrations of 0.1 μM, 1 UM and 10 μM. After 3 days in culture, 100 μl fresh media with the appropriate concentration of compound was added to each well. Cells were incubated for a further 4 days (total treatment duration 7 days, FIG. 10) and then 100 μl media was removed and added to 100 μl CellTitre-Glo® reagent (PROMEGA). The mixture was incubated at room temperature for 15 minutes with shaking and then the fluorescence of each well was quantified using a plate reader (Spectramax M5, MOLECULAR DEVICES) (FIG. 10).

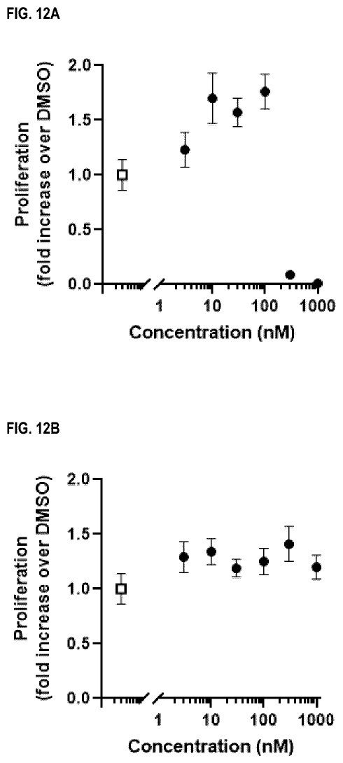

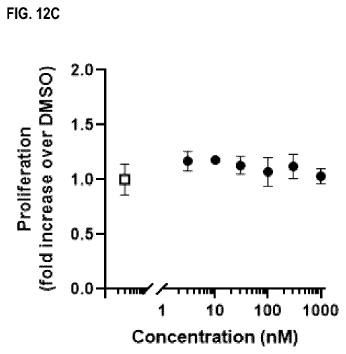

A number of compounds were toxic at all concentrations used in the assay, including Perturbagen 32, Perturbagen 33, Perturbagen 34, Perturbagen 35, Perturbagen 37, Perturbagen 40 and Perturbagen 43 (FIG. 11). However, we did identify a number of “hits” in which CD8+ T-cell number increased over DMSO control at the end of the exhaustion assay (7 days). We re-tested these “hits” at multiple concentrations (1 nM to 1000 nM) and found that Perturbagen 6 (FIG. 12A), Perturbagen 18 (FIG. 12B), and Perturbagen 12 (FIG. 12C) reproducibly and dose-dependently increased T-cell number in the T cell exhaustion assay (FIGS. 12A-12C). Perturbagens referenced in this Example refer to perturbagens of Table 4.

### Example 4: Generation of scRNAseq Data for Exhausted and Non-Exhausted CD8+ T Cells

Purified CD8+ T cells from 3 different donors (STEMCELL TECHNOLOGIES) were thawed into RPMI1640 media supplemented with FBS (10%), HEPES (10 mM), penicillin (50 units/ml)/streptomycin (50 μg/ml) and L-glutamate (2 mM). Cells were spun down (300 g, 5 minutes) and resuspended into culture media at a concentration of 1× 10⁶ cells/ml. Two milliliters of the cell suspension (2 million cells) was placed into 2 wells of a 6-well plate. Cells in well 1 were treated with 1× anti-CD3/CD28 (25 μl/ml) while cells in well 2 were treated with 6× anti-CD3/CD28 (150 μl/ml).

The remaining cells from the thaw were cryopreserved. These cells were thawed on the final day of the experiment and served as non-stimulated controls.

After 3 days of culture, the media was replaced with fresh media. On day 8, cells were harvested and counted. The cells were then spun down and resuspended into PBS to give a final concentration of 1×10⁶ cells/ml.

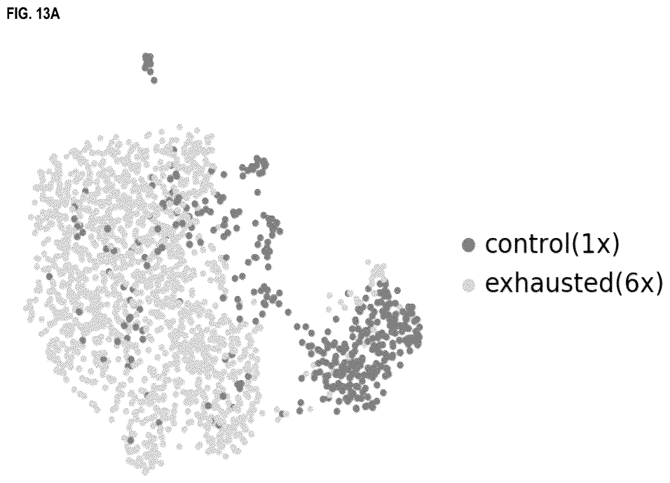

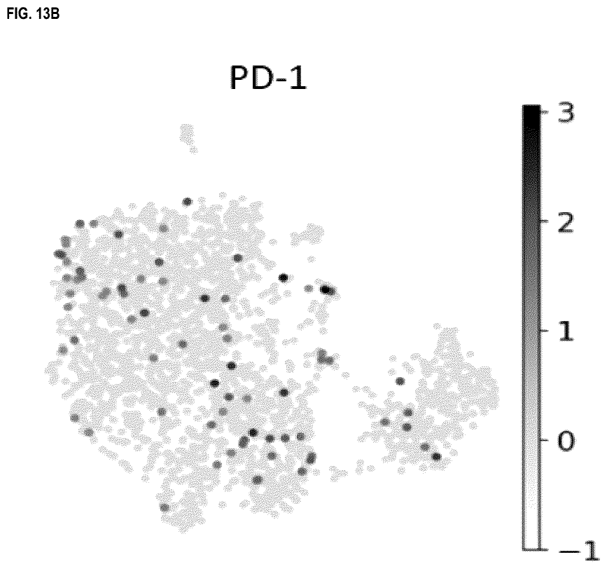

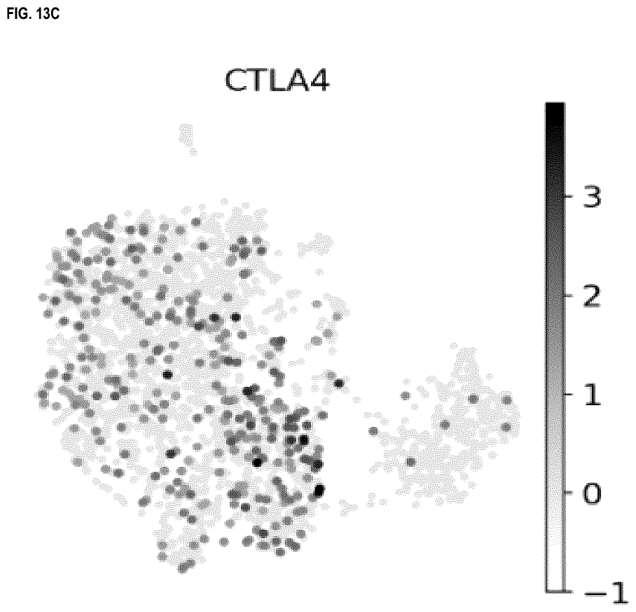

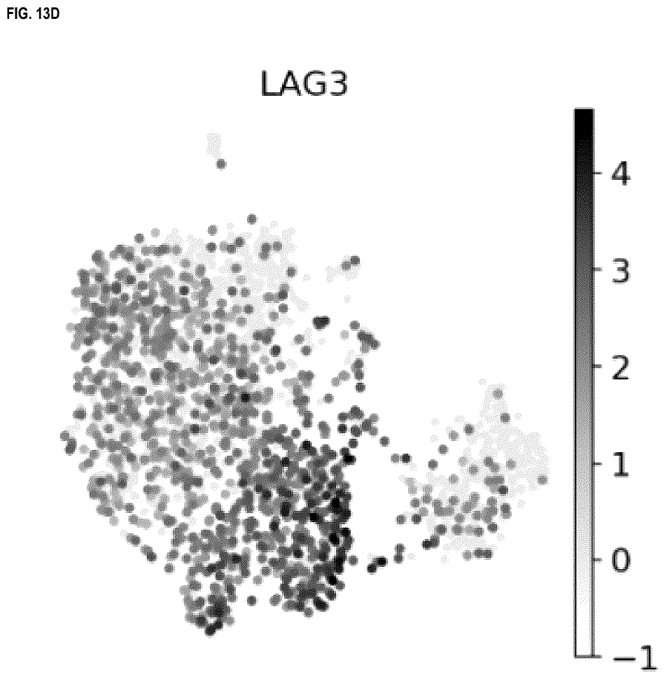

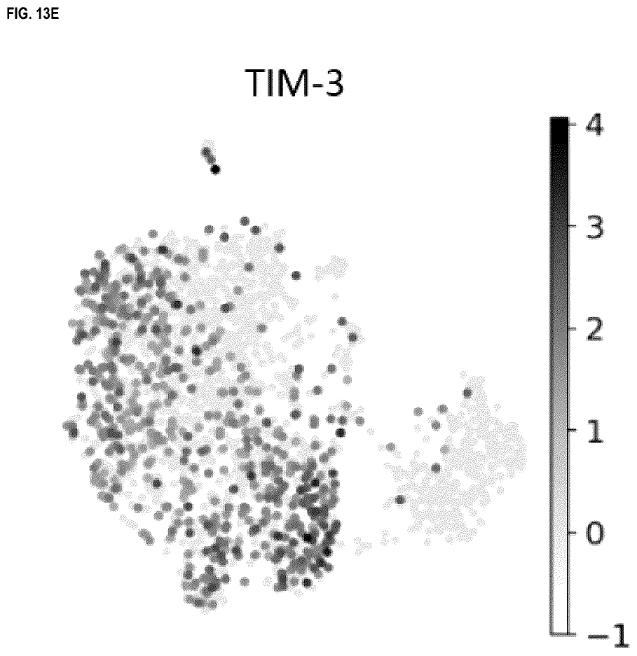

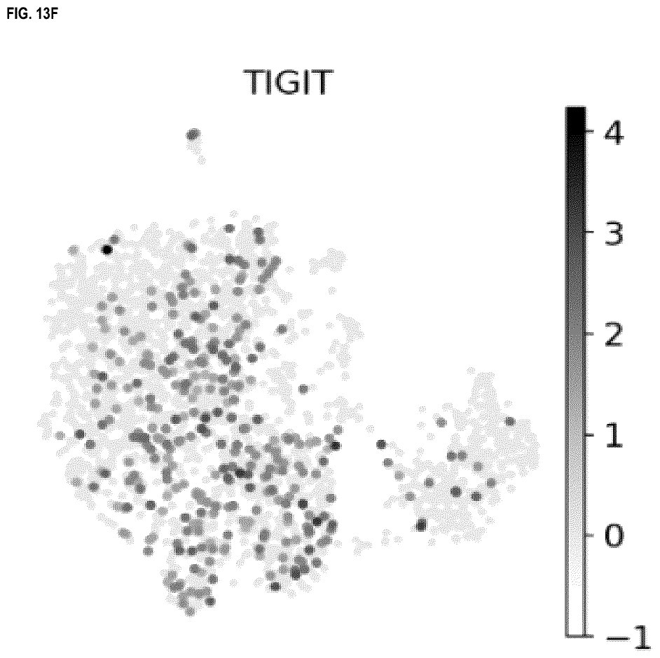

All samples were run on a 10× chromium box to generate shortread sequencer compatible libraries. Samples were subsequently sequenced separately using a NextSeq (ILLUMINA) with version 2.5 chemistry. Analysis of resulting single cell RNA datasets revealed distinct cell populations as a result of the T cell exhaustion protocol (FIG. 13A-13F).

### Example 5: Identification of T Cell Phenotype in a Cell Population Isolated from a Subject

The T cell phenotype of cells isolated from a subject can be identified using flow cytometry, e.g. before and/or after contacting with one or more perturbagens. For flow cytometry, samples of cells obtained from a subject can be incubated with an individual antibody or an antibody panel optimized for detection of effector and exhausted T cells as shown in Tables 9 and 10.

TABLE 9
Panel of Antibodies Used in Flow Cytometry of
Tumor Samples to Identify T-cell Phenotypes

| | | | Attune | | | |
|---|---|---|---|---|---|---|
| No. | Marker | Conjugate | Filter | Vendor | Catalog # | Clone |
| 1 | Viability | APC-eFluor 780 | RL3 | ThermoFisher | 65-0865-14 | N/A |
| 2 | CD45 | FITC | BL1 | ThermoFisher | 11-0451-82 | 30-F11 |
| 3 | CD3 | PE-Cy7 | YL4 | BioLegend | 100220 | 17A2 |
| 4 | CTLA-4 | PerCP-Cy5.5 | BL3 | BioLegend | 106316 | UC10-4B9 |
| 5 | CD8 | BV 510 | VL2 | Biolegend | 100752 | 53-6.7 |
| 6 | TCF7 | PE | YL1 | BD Biosciences | 564217 | S33-966 |
| 7 | PD-1 | PE-Dazzle 594 | YL2 | BioLegend | 135228 | 29F.1A12 |
| 8 | TIM-3 | BV 421 | VL1 | BD Biosciences | 747626 | 5D12 |
| 9 | TIGIT | BV 711 | VL4 | BD Biosciences | 744214 | 1G9 |
| 10 | LAG-3 | APC | RL1 | Miltenyi | 130-102-527 | C9B7W |
| 11 | CD25 | Alexa Fluor 700 | RL2 | BioLegend | 102024 | PC61 |

TABLE 10
Cell Types and Staining

| Marker | Cell type/Physiology |
|---|---|
| CD45 | Leucocyte marker |
| CD3 | T-cell marker |
| CTLA-4 | Inhibitory receptor |
| CD8 | Co-receptor of the T-cell receptor |
| TCF7 | Transcription factor that drives |
| | inhibitory receptor expression |
| PD-1 | Inhibitory receptor |
| TIM-3 | Inhibitory receptor |
| TIGIT | Inhibitory receptor |
| LAG-3 | Inhibitory receptor |
| CD25 | IL-2 receptor |

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

### EQUIVALENTS

While the disclosure has been disclosed in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

1.-70. (canceled)

71. A method of identifying a candidate perturbation for reducing or reversing the conversion of a starting population of T cells into exhausted T cells, the method comprising:
(a) exposing the starting population of T cells to a perturbation;
(b) identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the starting population of T cells into exhausted T cells following exposure of the population of cells to the perturbation; and
(c) identifying the perturbation as a candidate perturbation for reducing or reversing the conversion of the starting population of T cells thereof based on the perturbation signature;
wherein the perturbation signature is an increase in expression and/or activity in the starting population of T cells of one or more genes selected from Table 1 or Table 2 designated as an “up” gene in the gene directionality column of Table 1 or Table 2, and/or a decrease in expression and/or activity in the starting population of T cells of one or more genes selected from Table 1 or Table 2 designated as a “down” gene in the gene directionality column of Table 1 or Table 2.

72. The method of claim 71, wherein the perturbation signature comprises an activation of a network module designated in the network module column of Table 1 or Table 2.

73. The method of claim 71, wherein the perturbation signature is an increase in expression and/or activity in the starting population of T cells of one or more genes selected from Table 1 or Table 2 designated as an “up” gene in the gene directionality column of Table 1 or Table 2.

74. The method of claim 71, wherein the perturbation signature is a decrease in expression and/or activity in the starting population of T cells of one or more genes selected from Table 1 or Table 2 designated as a “down” gene in the gene directionality column of Table 1 or Table 2.

75. The method of claim 72, wherein the activation of the network module designated in the network module column of Table 1 or Table 2 comprises modulating expression and/or activity of 2 or more genes within a network module.

76. The method of claim 72, wherein the activation of the network module designated in the network module column of Table 1 or Table 2 comprises modulating expression and/or activity of all of the genes within a network module.

77. The method of claim 72, wherein the activation of the network module designated in the network module column of Table 1 or Table 2 comprises modulating expression and/or activity of 2 or more genes within 2 or more network modules.

78. The method of claim 71, wherein the one or more genes selected from Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, and 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more genes are selected from Table 1 designated as an “up” gene in the gene directionality column of Table 1.

79. The method of claim 71, wherein the one or more genes selected from Table 1 comprises at least one of NFKBIA, TSC22D3, ZFP36, ARHGEF2, FHL2, STMN1, CDC25B, CCND3, TMEM109, E2F2, SCP2, PDLIM1, CORO1A, ATP11B, SATB1, CXCR4, ARL4C, CTSD, CD44, ZMIZ1, TBXA2R, GNA15, PRKCQ, RHOA, SLC25A4, PRUNE, CDC42, TIMP2, FAM69A, NRAS, BHLHE40, DNAJC15, GNAI2, DHRS7, CYTH1, ADGRE5, IGF2R, ADRB2, EIF4EBP1, FAS, MRPS16, TMEM50A, S100A4, RSU1, SPTAN1, S100A13, RAC2, REEP5, MACF1, PLP2, and TWF2.

80. The method of claim 71, wherein the one or more genes selected from Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, and 20 or more, 21 or more, 22 or more, 23 or more, 24 or more, 25 or more, 26 or more, 27 or more, 28 or more, 29 or more, 30 or more, or 31 or more genes selected from Table 1 designated as a “down” gene in the gene directionality column of Table 1.

81. The method of claim 71, wherein the one or more genes selected from Table 1 comprises at least one of STAT1, DUSP6, INPP1, PSMB8, MLEC, ID2, RGS2, UBE2L6, SSBP2, PRKCH, ALDOA, ADGRG1, MFSD10, HERC6, CEP57, FBXL12, ICAM1, GLRX, PSME2, MYCBP2, IKZF1, PSMB10, PSME1, EVL, MBNL1, FYN, DNAJB6, FOXO3, TSPAN3, SYNE2, and RPS6.

82. The method of claim 71, wherein the one or more genes selected from Table 2 designated as an “up” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, and 19 or more, 20 or more, 21 or more, 22 or more, 23 or more, 24 or more genes selected from Table 2 designated as an “up” gene in the gene directionality column of Table 2.

83. The method of claim 71, wherein the one or more genes selected from Table 2 comprises at least one of MYC, TES, CXCR4, IGFBP3, PRSS23, SYPL1, CYB561, CCNH, XBP1, RPS6, ADRB2, GDPD5, SORBS3, ZFP36, FOS, PXN, SLC25A4, DSG2, SATB1, IER3, SSBP2, RPS5, ATP1B1, and GADD45B.

84. The method of claim 71, wherein the one or more genes selected from Table 2 designated as a “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, and 62 or more genes selected from Table 2 designated as a “down” gene in the gene directionality column of Table 2.

85. The method of claim 71, wherein the one or more genes selected from Table 2 comprises at least one of CDK6, MTHFD2, ID2, SCCPDH, SLC25A46, ETFB, HLA-DRA, CHN1, RAB27A, TBXA2R, NFKB2, ITGAE, SMC4, STMN1, GATA3, ETS1, IQGAP1, CAT, RALA, TSC22D3, CBLB, INPP4B, PLSCR1, NUSAP1, RGS2, EVL, PSMB8, HERPUD1, APBB2, MIF, SQSTM1, PGAM1, TWF2, DRAP1, ETV1, CCNA1, HTRA1, DUSP4, GAPDH, RPA3, ADGRG1, ACOT9, CALM3, SOX4, HMOX1, RHOA, S100A4, ANKRD10, FCHO1, KDM5B, SPTAN1, CTSD, HLA-DMA, FGFR4, SLC1A4, HSPB1, CDKN2A, STAT3, RAC2, TIAM1, RALGDS, and EZH2.

86. A method for making a therapeutic agent for a cancer or infection, comprising:
(a) identifying a candidate perturbation according to the method of claim 71, and
(b) formulating the candidate perturbation as a therapeutic agent for the treatment of the cancer or infection.

87. The method of claim 86, wherein the cancer is a solid tumor or a liquid tumor, optionally wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

88. The method of claim 86, wherein the infection is selected from bacterial infections, viral infections, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.
